(19) United States
(12) Patent Application Publication
(10) Pub. No.: US 2012/0100628 A1
(43) Pub. Date: Apr. 26, 2012

(54) EMISSIVE AND BROADBAND NONLINEAR ABSORBING METAL COMPLEXES AND LIGANDS AS OLED, OPTICAL SWITCHING OR OPTICAL SENSING MATERIALS

(75) Inventors: Wenfang Sun, Fargo, ND (US); Bingguang Zhang, Wuhan (CN); Rui Liu, Fargo, ND (US); Pin Shao, Pittsburgh, PA (US); Zhiqiang Ji, Columbus, OH (US); Zhongjing Li, Fargo, ND (US)

(73) Assignee: NDSU RESEARCH FOUNDATION, Fargo, ND (US)

(21) Appl. No.: 13/278,780

(22) Filed: Oct. 21, 2011

Related U.S. Application Data
(60) Provisional application No. 61/405,387, filed on Oct. 21, 2010.

(51) Int. Cl. G01N 21/78; C07D 417/14; G02B 6/26; C07D 471/04; H01L 51/50; C07F 15/00; C07D 401/04

(52) U.S. Cl. 436/169; 546/10; 546/256; 546/257; 546/88; 257/40; 385/16; 257/E51.024

(57) ABSTRACT

Platinum (II) terdentate or bidentate complexes with non-linear optical properties are provided. The complexes have a broadband spectral and temporal response, and strong reverse saturable absorption and two-photon absorption in the visible and the near-IR region. As such, the complexes are useful for organic light-emitting diodes and optical-switching or sensing devices.

### CROSS-REFERENCE TO RELATED APPLICATIONS

This application claims the benefit of U.S. Provisional Application Ser. No. 61/405,387, filed Oct. 21, 2010 which is incorporated by reference herein.

### STATEMENT REGARDING FEDERALLY SPONSORED RESEARCH

This invention was made with government support under grant CHE-0449598 awarded by the National Science Foundation (NSF), and grants W911NF-06-2-0032 and W911NF-10-2-0055 awarded by the Army Research Lab. The Government has certain rights in this invention.

### BACKGROUND

Certain organic compounds can exhibit nonlinear optical properties, e.g., nonlinear absorption and/or nonlinear refraction, when excited with a laser. However, the absorption cross-section or triplet excited-state lifetime of conventional organic compounds can be limited, which makes it difficult to realize applications such as organic light-emitting diodes, and optical-switching or optical-sensing devices with conventional organic compounds.

### SUMMARY

In one aspect, the invention provides a ligand of formula (I):

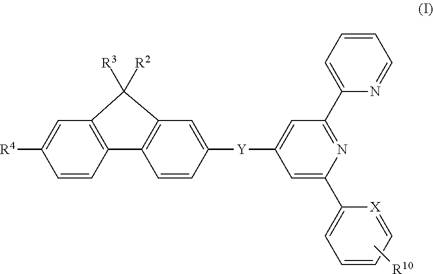

wherein:

R¹⁰ is H or —OR^{a};

R² and R³ are each independently C₁-C₂₄ alkyl, C₂-C₂₄ alkenyl, C₂-C₂₄ alkynyl or —(O—CH₂—CH₂)ₙ—OCH₃;

R⁴ is H, halo, aryl, heterocyclyl, arylalkyl, heterocyclylalkyl, arylalkynyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f};

X is C or N;

each n is independently an integer from 1-12;

each R^{a}, R^{b}, R^{c}, R^{d}, R^{e}, and R^{f} is independently selected from H, C₁-C₂₄ alkyl, C₂-C₂₄ alkenyl or C₂-C₂₄ alkynyl, aryl, heterocyclyl, arylalkyl, and heterocyclylalkyl; and

Y is a bond, —CH═CH—, —CH═CH-Ph-, or —C≡C—;

wherein when the ligand of formula (I) bears a charge, it further comprises one or more counterions.

In another aspect, the invention provides a ligand of formula (II):

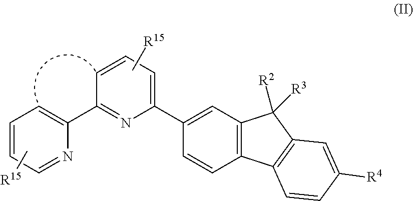

wherein:

the dashed line represents the presence or absence of an optionally substituted aromatic ring or fused aromatic rings;

R² and R³ are each independently C₁-C₂₄ alkyl, C₂-C₂₄ alkenyl, C₂-C₂₄ alkynyl or —(O—CH₂—CH₂)ₘ—OCH₃;

R⁴ is H, halo, aryl, heterocyclyl, arylalkynyl, arylalkyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl, or —CH═N—NH—R^{f};

each R¹⁵ is independently selected from H, halo, aryl, heterocyclyl, arylalkynyl, C₁-C₂₄ alkyl, C₂-C₂₄ alkenyl, or C₂-C₂₄ alkynyl;

each m is independently an integer from 1-12; and

each R^{b}, R^{c}, R^{d}, R^{e}, and R^{f} is independently selected from H, C₁-C₂₄ alkyl, C₂-C₂₄ alkenyl, C₂-C₂₄ alkynyl, heterocyclyl, arylalkyl, heterocyclylalkyl and aryl,

wherein when the ligand of formula (II) bears a charge, it further comprises one or more counterions.

In yet another aspect, the invention provides a ligand of formula (III):

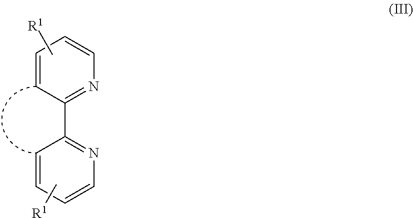

wherein:

each R¹ is independently a group of the following formula:

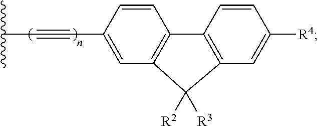

each n equals to 0 or 1;

each R² and R³ is independently C₁-C₂₄ alkyl, C₂-C₂₄ alkenyl or C₂-C₂₄ alkynyl, or —(O—CH₂—CH₂)ₘ—OCH₃;

each R⁴ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f}; and

each R^{b}, R^{c}, R^{d}, R^{e}, and R^{f} is independently selected from H, C₁-C₂₄ alkyl, C₂-C₂₄ alkenyl, C₂-C₂₄ alkynyl, heterocyclyl, arylalkyl, heterocyclylalkyl and aryl;

each m is independently an integer from 1-12; and

wherein the dashed line represents the presence or absence of an optionally substituted aromatic ring or fused aromatic rings; and

wherein when the ligand of formula (III) bears a charge, it further comprises one or more counterions.

In a further aspect, the invention provides a metal complex of formula (IV):

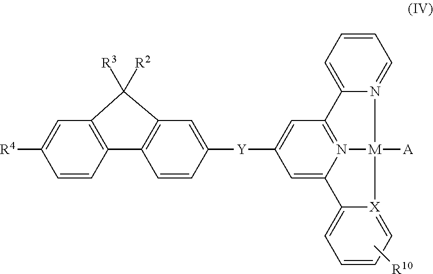

wherein:

R¹⁰ is H or —OR^{a};

R² and R³ are each independently C₁-C₂₄ alkyl, C₂-C₂₄ alkenyl, C₂-C₂₄ alkynyl or —(O—CH₂—CH₂)ₘ—OCH₃;

R⁴ is H, halo, aryl, heterocyclyl, arylalkyl, heterocyclylalkyl, arylalkynyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f};

A is selected from halo,

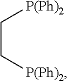

### and —C≡C—R^{g};

X=C or N;

each m is independently an integer from 1-12;

each R^{a}, R^{b}, R^{c}, R^{d}, R^{e}, R^{f}, and R^{g} is independently selected from H, C₁-C₂₄ alkyl, C₂-C₂₄ alkenyl or C₂-C₂₄ alkynyl, aryl, heterocyclyl, arylalkyl, arylalkynyl and heterocyclylalkyl; and

Y is a bond, —CH═CH—, —CH═CH-Ph-, or —C≡C—;

M is a metal ion; and

wherein when the complex of formula (IV) bears a charge, it further comprises one or more counterions.

In one aspect, the invention provides a metal complex of formula (V):

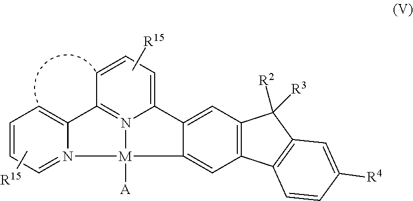

wherein:

the dashed line represents the presence or absence of an optionally substituted aromatic ring or fused aromatic rings;

R² and R³ are each independently C₁-C₂₄ alkyl, C₂-C₂₄ alkenyl, C₂-C₂₄ alkynyl or —(O—CH₂—CH₂)ₘ—OCH₃;

R⁴ is H, halo, aryl, heterocyclyl, arylalkynyl, arylalkyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl, or —CH═N—NH—R^{f};

R¹⁵ is each independently selected from H, halo, aryl, heterocyclyl, arylalkynyl, C₁-C₂₄ alkyl, C₂-C₂₄ alkenyl, or C₂-C₂₄ alkynyl;

A is selected from halo and —C≡C—R^{g};

M is a metal ion;

each m is independently an integer from 1-12; and

each R^{b}, R^{c}, R^{d}, R^{e}, R^{f}, and R^{g} is independently selected from H, C₁-C₂₄ alkyl, C₂-C₂₄ alkenyl, C₂-C₂₄ alkynyl, heterocyclyl, arylalkyl, arylalkynyl, heterocyclylalkyl and aryl,

wherein when the complex of formula (V) bears a charge, it further comprises one or more counterions.

In another aspect, the invention provides a metal complex of formula (VI):

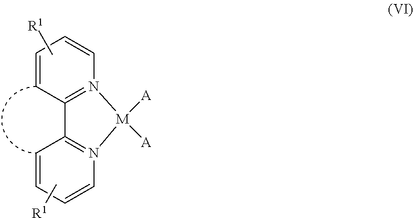

wherein:

the dashed line represents the presence or absence of an optionally substituted aromatic ring or fused aromatic rings;

each R¹ is independently a group of the following formula:

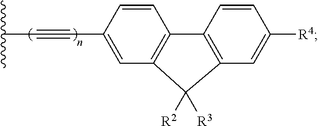

each n equals to 0 or 1

each R² and R³ is independently C₁-C₂₄ alkyl, C₂-C₂₄ alkenyl, C₂-C₂₄ alkynyl, or —(O—CH₂—CH₂)ₘ—OCH₃;

each m is independently an integer from 1-12;

each R⁴ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f}; and

each R^{b}, R^{c}, R^{d}, R^{e}, and R^{f} is independently selected from H, C₁-C₂₄ alkyl, C₂-C₂₄ alkenyl, C₂-C₂₄ alkynyl, heterocyclyl, arylalkyl, heterocyclylalkyl and aryl;

A is selected from halo and —C≡C—R^{g};

each R^{g} is independently selected from H, C₁-C₂₄ alkyl, C₂-C₂₄ alkenyl, C₂-C₂₄ alkynyl, heterocyclyl, arylalkyl, arylalkynyl, heterocyclylalkyl and aryl; and

M is a metal ion;

wherein when the complex of formula (VI) bears a charge, it further comprises one or more counterions.

In a further aspect, the invention provides a metal complex of formula (VII):

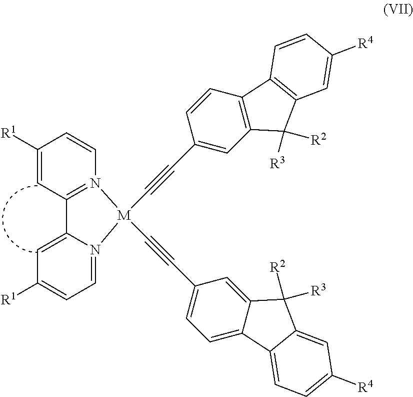

wherein

M is a metal ion;

the dashed line represents the presence or absence of an optionally substituted aromatic ring or fused aromatic rings;

each R¹ is independently C₁-C₂₄ alkyl, C₂-C₂₄ alkenyl, C₂-C₂₄ alkynyl, arylalkynyl or —(O—CH₂—CH₂)ₘ—OCH₃;

each R² and R³ is independently C₁-C₂₄ alkyl, C₂-C₂₄ alkenyl, C₂-C₂₄ alkynyl, or —(O—CH₂—CH₂)ₙ—OCH₃;

each R⁴ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f};

each m is independently an integer from 1-12;

each R^{b}, R^{c}, R^{d}, R^{e}, and R^{f} is independently selected from H, C₁-C₂₄ alkyl, C₂-C₂₄ alkenyl, C₂-C₂₄ alkynyl, arylalkyl, heterocyclylalkyl and aryl; and

wherein when the complex of formula (VII) bears a charge, it further comprises one or more counterions.

In another aspect, the invention provides a metal complex of formula (VIII):

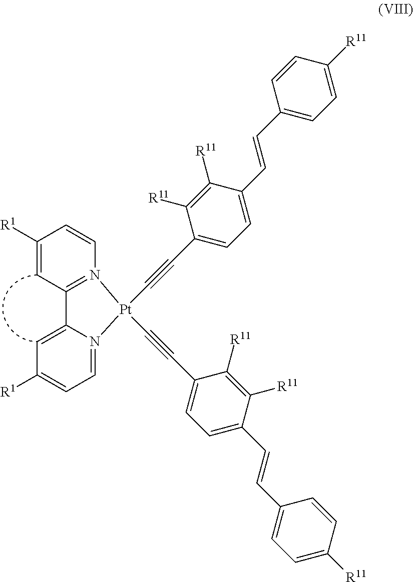

wherein

M is a metal ion;

the dashed line represents the presence or absence of an optionally substituted aromatic ring or fused aromatic rings;

each R¹ is independently C₁-C₂₄ alkyl, C₂-C₂₄ alkenyl, C₂-C₂₄ alkynyl, arylalkynyl or —(O—CH₂—CH₂)ₘ—OCH₃;

each R¹¹ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, and C₂-C₂₄ alkynyl;

each m is independently an integer from 1-12;

each R^{b}, R^{c}, R^{d}, and R^{e} is independently selected from H, C₁-C₂₄ alkyl, C₂-C₂₄ alkenyl, C₂-C₂₄ alkynyl, arylalkyl, heterocyclylalkyl and aryl; and

wherein when the complex of formula (VIII) bears a charge, it further comprises one or more counterions.

In another aspect, the invention provides optical-switching devices, organic light emitting diodes, chemical sensors such as for organic vapors, ion sensors such as for zinc, and pH sensors using the ligands and metal complexes described herein.

Other aspects of the invention will become apparent by consideration of the detailed description and accompanying drawings.

### BRIEF DESCRIPTION OF THE DRAWINGS

FIG. 1 depicts various ligands and complexes according to the present invention.

FIG. 2 shows UV-vis absorption spectra of 5×10⁻⁵ mol/L dichloromethane solutions of 3, 4, 5, F-3 and F-4.

FIG. 3 shows UV-vis absorption spectra of 5×10−5 mol/L solution of 4 in different solvents measured in a 1-cm cuvette at room temperature.

FIG. 4 shows concentration-dependent emission spectra of 5 (λex=479 nm) in toluene at room temperature.

FIG. 5 shows emission spectra of 5 measured in degassed butyronitrile glassy solution at 77 K.

FIG. 6 shows (a) triplet transient difference absorption spectra of 3-5, F-3 and F-4 at zero time delay. 3, F-3 and F-4 were measured in degassed CH₃CN solution, and 4 and 5 were measured in degassed toluene solution. λex=355 nm and Aabs=0.40 at the excitation wavelength; and (b) time-resolved triplet transient difference absorption spectra of 5 in degassed toluene solution following 355 nm excitation with Aabs=0.40. The time listed in the figure is the time delay after the excitation.

FIG. 7 shows transmission vs incident fluence curves for 4 and 5 in toluene solutions for 4.1 ns laser pulses at 532 nm in a 2-mm cell. The linear transmission was adjusted to 80%.

FIG. 8 shows surface pressure-mean molecular area isotherms for 4 and 5.

FIG. 9 shows AFM height imagines of 5-layer (left) and 11-layer (right) LB films of 4 and 5. The scan area was 1 μm×1 μm, and the Z-range was 200 nm.

FIG. 10 shows UV-vis absorption spectra of 4 and 5 in LB films and in toluene solution.

FIG. 11 shows emission spectra of 4 and 5 in butyronitrile glassy solutions at 77 K, in toluene solutions at room temperature and in LB films at room temperature when excited at 355 nm. The asterisks indicate instrument artifact.

FIG. 12 shows UV-vis absorption spectra of F-5-F-9 in CH₂Cl₂. c=5×10−5 mol/L. The inset shows the expansion of the charge transfer band.

FIG. 13 shows UV-vis absorption spectra of F-8 in different solvents. c=5×10−5 mol/L.

FIG. 14 shows normalized emission spectra of F-5-F-9 in CH₂Cl₂ at a concentration of 5×10⁻⁵ mol/L. The excitation wavelength is 440 nm.

FIG. 15 shows emission spectra of F-9 in CH₂Cl₂ at room temperature with different excitation wavelengths.

FIG. 16 shows emission spectra of F-8 in different solvents at room temperature. c=5×10⁻⁵ mol/L. λₑₓ=440 nm.

FIG. 17 shows the normalized emission spectra of F-9 at different concentrations in CH₂Cl₂ at room temperature. λₑₓ=441 nm.

FIG. 18 shows emission spectra of F-5 at room temperature and at 77 K. ₑₓ=423 nm. c=5×10⁻⁵ mol/L.

FIG. 19 shows (a) triplet transient difference absorption spectra of F-5-F-8 in argon-degassed CH₃CN solution at room temperature at zero time delay following 355 nm excitation; and (b) time-resolved TA spectra for F-6 in degassed CH₃CN solution.

FIG. 20 shows transmittance vs. incident fluence curves of F-5-F-9 in CH₂Cl₂ for 4.1 ns laser pulses at 532 nm in a 2-mm cell. The linear transmission was adjusted to 80%.

FIG. 21 shows ns and ps open-aperture Z-scan experimental data and fitting curves for F-5 at 532 nm. The energy used was 3.9 μJ for ns Z scan and 1.5 μJ for ps Z scan, and the beam radius at the focal point was 30 μm for ns Z scan and 34 μm for ps Z scan.

FIG. 22 shows UV-Vis absorption spectra of 17, 18, and 19 in CH₂Cl₂ at a concentration of 1×10⁻⁵ mol/L.

FIG. 23 shows (a) UV-vis spectra of 19 in CH₂Cl₂ with addition of p-TsOH/CH₃CN solution; and (b) UV-Vis spectra of 19 in different solvents with addition of 3 equiv. of p-TsOH. (c=4.9×10⁻⁵ mol/L).

FIG. 24 shows UV-vis absorption spectra of F-10, F-11, and F-12 in CH₃CN at a concentration of 1×10⁻⁵ mol/L.

FIG. 25 shows normalized emission spectra of 17 (λₑₓ=395 nm), 18 (λₑₓ=390 nm), and 19 (λₑₓ=395 nm) in CH₂Cl₂ (c=1×10⁻⁵ mol/L).

FIG. 26 shows (a) normalized emission spectra of F-10 (λₑₓ=384 nm), F-11 (λₑₓ=374 nm), and F-12 (λₑₓ=389 nm) in CH₃CN at room temperature (c=1×10⁻⁵ mol/L); and (b) emission spectra of F-11 at different excitation wavelengths (c=5×10⁻⁵ mol/L).

FIG. 27 shows (a) concentration-dependent emission spectra of F-11 in CH₃CN solutions at room temperature; and (b) normalized UV-Vis and emission spectra of F-11 in CH₃CN (c=1×10⁻⁵ mol/L).

FIG. 28 shows room temperature emission spectra of 19 (c=5.3×10⁻⁵M) in CH₂Cl₂ with addition of p-TsOH/CH₃CN solution. λₑₓ=396 nm.

FIG. 29 shows emission spectra of F-10-F-12 at 77 K in butyronitrile. (c=1×10⁻⁵ mol/L, λₑₓ=355 nm).

FIG. 30 shows time-resolved triplet transient difference absorption spectra of 17 in CH₂Cl₂. λₑₓ=355 nm. The concentration of the solution was adjusted to obtain A=0.4 at 355 nm in a 1-cm cuvette.

FIG. 31 shows time-resolved femtosecond transient difference absorption spectra and ground-state absorption spectrum of F-10 in CH₃CN. λₑₓ=400 nm.

FIG. 32 shows two-photon absorption spectra (symbols) and one-photon absorption spectra (solid lines) of 17, 18, and 19 in toluene. The experimental error for the TPA spectra measurement is approximately ±30%.

FIG. 33 shows open-aperture Z-scan experimental data and fitting curves for F-11 in CH₃CN at different wavelengths. The energy used for the experiment was 2.7 μJ at 575 nm and 6.6 μJ at 740 nm, and the beam waist at the focal point was 31 μm.

FIG. 34 shows UV-Vis absorption spectra of 29 and F-14 in CH₂Cl₂.

FIG. 35 shows normalized emission spectra of 29 and F-14 in CH₂Cl₂ solutions at room temperature and in butyronitrile matrix at 77 K for F-14.

FIG. 36 shows time-resolved singlet transient difference absorption spectra of 29 and F-14 in CH₂Cl₂. λₑₓ=400 nm.

FIG. 37 shows time-resolved triplet transient difference absorption spectra of 29 in butyronitrile and F-14 in CH₃CN. λₑₓ=355 nm.

FIG. 38 shows plots of Z-scan experimental data (symbols) and fitting curves (solid lines) for F-14 in CH₂Cl₂ solution at 532 nm and 680 nm in a 2-mm cell. The spot size at the focal plane is 33 μm for ps pulses and 37 μm for ns pulses at 532 nm, and 38 μm for ps pulses at 680 nm. The energy used is 5.20 for ns measurement and 3.10 for ps measurement at 532 nm, and 10.70 for ps measurement at 680 nm. The solution concentration is 6.72×10⁻⁴ mol/L and 3.82×10⁻⁴ mol/L for the ns and ps measurements at 532 nm, respectively; and 4.71×10⁻³ mol/L for the 680-nm measurement.

FIG. 39 shows nonlinear transmission of F-14 in CH₂Cl₂ solution for 4.1 ns laser pulses at 532 nm. The linear transmission is 80%, and the path length of the cuvette is 2 mm. The inset show the fractional population of the affected excited states on the input face of the sample within a laser pulse. The fluence used for the calculation is 0.24 J/cm².

FIG. 40 shows normalized emission spectra of complex F-15 in different solvents at room temperature and in butyronitrile glassy matrix at 77 K.

FIG. 41 shows the T₁-Tₙ transient difference absorption spectra of complex F-15 in CH₂Cl₂ and 35 in butyronitrile at zero time delay after the excitation at 355 nm.

FIG. 42 shows time-resolved fs transient difference absorption spectrum of complex F-15 in CH₂Cl₂ solution at room temperature.

FIG. 43 shows picosecond Z-scan experimental data (symbols) and fitting curves (solid lines) for complex F-15 in CH₂Cl₂ solution at 532 nm and 760 nm with different excitation energies.

FIG. 44 shows fractional distribution of the populations of the affected excited states during a 21 ps laser pulse at 532 nm and 760 nm at the input face of the sample solution.

FIG. 45 shows two-photon absorption spectra (symbols) and one-photon absorption spectra (solid lines) of complex F-15 in CH₂Cl₂.

FIG. 46 shows transmission vs. incident fluence curve for complex F-15 in CH₂Cl₂ solution at 532 nm using 4.1 ns laser pulses.

FIG. 47 shows fractional distribution of the populations of the affected excited states during a 4.1 ns laser pulse at 532 nm at the input face of the sample solution.

FIG. 48 shows linear absorption spectra: (a) UV-vis absorption of ligands used for compounds F-16, F-19-F-21; (b) UV-vis absorption spectra of Pt-complexes F-16-F-21; (c) calculated absorption spectra for the complexes F-16-F-21 vertical lines resemble excited states and the corresponding oscillator strength.

FIG. 49 shows solvatochromic effects of absorption of the complexes F-19 and F-16. Left: experimental (a) and calculated (b) absorption of complex F-19 in solvents of different polarity; Right: experimental (c) and calculated (d) absorption spectra of F-16 in different solvents. Arrows indicate the blue-shift of the absorption bands with increasing solvent polarity.

FIG. 50 shows normalized emission of (a) ligands 36, 39, 40, and 41 and (b) complexes F-16-F-21 in dichloromethane at room temperature; (c) complexes F-16-F-21 in butylnitrile glass at 77 K (phosphorescence).

FIG. 51 shows ns transient absorption of complexes F-16-F-21 in acetonitrile at room temperature.

FIG. 52 shows time-resolved triplet transient difference absorption spectra of complex F-21 in acetonitrile.

FIG. 53 shows nonlinear optical properties of complexes F-16-F-21 in dichloromethane.

FIG. 54(a) shows UV-vis absorption spectrum of complex F-15 in CH₂Cl₂ solution, with the inset showing the normalized UV-vis absorption spectra of F-15 and 35 in CH₂Cl₂ solution.

FIG. 54(b) shows expansion of the UV-vis spectrum of F-15 between 520 nm and 600 nm in CH₂Cl₂.

### DETAILED DESCRIPTION

Metal complexes, including platinum and zinc complexes, having ligands bearing substituted fluorenyl substituents are described herein. These complexes may exhibit broad and strong reverse saturable absorption in the visible spectral region and two-photon absorption in the near-IR region, and may have good solubilities in organic solvents. In addition, various ligands are described herein. The complexes and the ligands may be useful as components of optical-switching devices, organic light emitting diodes (OLED), and chemical sensors, such as pH sensors and zinc sensors. The emission properties and nonlinear transmission properties of the metal complexes can be tuned by introducing different substituents on the terdentate (i.e. terpyridine (N̂N̂N) or phenylbipyridine (ĈN̂N)) or diimine (i.e. bipyridine (N̂N)) ligand and using different co-ligands. They can also be altered through inter-/intra-molecular interactions, such as the metal-metal or π-π interactions. Though not wishing to be bound by a particular theory, the unique photophysical properties and the variety applications of the metal complexes could be due to their square-planar configuration, the intramolecular charge transfer characteristics, and the heavy-atom effect from the metal that enhances the intersystem crossing (ISC) rate to the triplet excited state.

In addition to emission studies, another powerful tool in understanding the excited-state characteristics of these complexes is the transient absorption measurement of the excited state, which can predict the nonlinear absorption of the compound. Time-resolved transient difference absorption study not only provides valuable information on the excited-state absorption spectrum but also on the lifetime of the excited state. In general, a positive band in a transient absorption spectrum suggests stronger excited-state absorption than that of the ground state in the respective spectral region, which could cause reverse saturable absorption.

The nonlinear absorption of the square-planar metal terpyridyl, phenylbipyridyl or diimine complexes would also be useful for numerous other applications wherein the following characteristics are desired: a broadband excited-state absorption, a long-lived triplet excited state, an ease of structural modification, and a thermal and photochemical stability of complexes.

In addition, metal complexes such as those of the present invention can be fabricated as Langmuir-Blodgett (LB) films. Due to the highly ordered nature of the LB films, the intermolecular interaction of the metal complexes in LB films may be different from that in solutions. Moreover, for potential device applications, it is important to be able to transfer the complexes into thin film forms. An LB film is a convenient method to fabricate highly ordered thin films. The presence of a long chain alkoxyl substituent on the ĈN̂N ligand could make the complex amphiphilic, which allows for preparation of LB films of the complex.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

### DEFINITIONS

As used herein, “alkyl” refers to a straight chain or branched, noncyclic or cyclic, saturated aliphatic hydrocarbon chain containing the indicated number of carbon atoms. Representative saturated straight chain alkyl groups include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. An alkyl group may be substituted or unsubstituted (e.g., by one or more substituents).

As used herein, “alkenyl” refers to an alkyl, as defined above, containing at least one double bond between adjacent carbon atoms. Alkenyls include both cis and trans isomers. Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. An alkenyl group may be substituted or unsubstituted (e.g., by one or more substituents).

As used herein, “alkynyl” refers to any alkyl or alkenyl, as defined above, which additionally contains at least one triple bond between adjacent carbons. Representative straight chain and branched alkynyls include acetylenyl, propynyl, 1-butynyl, 2-butynyl, 1-pentynyl, 2-pentynyl, 3-methyl-1-butynyl, and the like. An alkynyl group may be substituted or unsubstituted (e.g., by one or more substituents).

As used herein, an “aryl” group is an aromatic monocyclic, bicyclic, tricyclic or tetracyclic hydrocarbon ring system, wherein any ring atom capable of substitution can be substituted (e.g., by one or more substituents). Examples of aryl moieties include, but are not limited to, substituted or unsubstituted phenyl, naphthyl, anthracenyl, phenanthracenyl, fluorenyl and pyrenyl. An aryl moiety may also be a “heteroaryl” moiety. Heteroaryl refers to an aromatic monocyclic, bicyclic, tricyclic or tetracyclic ring system having at least one heteroatom selected from O, N, or S. Any ring atom capable of substitution can be substituted (e.g., by one or more substituents). Exemplary heteroaryls include, but are not limited to, furanyl, thienyl, thiazolyl, pyrrolyl, imidazolyl, pyrazolyl, triazolyl, oxazolyl, isoxazolyl, thiazolyl, isothiazolyl, oxadiazolyl, tetrazolyl, oxadiazolyl, oxatriazolyl, isoxazinyl, pyridyl, pyridazinyl, pyrimidyl, pyrazinyl, pyrimidyl, benzofuranyl, isobenzofuranyl, indolyl, isoindolyl, purinyl (e.g., adenine, guanine), benzothiophenyl, benzimidazolyl, quinolinyl, isoquinolinyl, benzodiazinyl, pyridopyridinyl, quinoxalinyl, carbazolyl, dibenzothiophenyl, dibenzofuranyl, acridinyl, phenazinyl, benzothiazolyl, phenothiazinyl, naphthalimide, and naphthalene diimide.

As used herein, an “arylalkyl” group refers to an alkyl moiety in which an alkyl hydrogen atom is replaced by an aryl group. Arylalkyl includes groups in which more than one hydrogen atom has been replaced by an aryl group. Examples of arylalkyl groups include, but are not limited to, benzyl, 2-phenylethyl, 3-phenylpropyl, 9-fluorenyl, benzhydryl, and trityl groups.

As used herein, the term “counterion” refers to an atom or group having a formal charge that is present to balance the charge of an ionic species in order to maintain electronic neutrality. Counterions can be positively charged (cations) or negatively charged (anions). Exemplary negatively charged counterions include halides (e.g., fluoride, chloride, bromide and iodide), N₃⁻, PO₄³⁻, HPO₄²⁻, H₂PO₄⁻, SO₄²⁻, HSO₄⁻, NO₃⁻, ClO₄⁻, CO₃², HCO₃⁻, CrO₄²⁻, Cr₂O₇²⁻, CN⁻, OH⁻, Cr₂O₄²⁻, MnO₄⁻, BF₄⁻, B(C₆H₅)₄⁻, PF₆⁻, HCOO⁻, CH₃COO⁻, CF₃COO⁻, CF₃SO₃⁻, PtCl₄²⁻, and the like.

As used herein, “halo” is fluoro, chloro, bromo or iodo.

As used herein, “heterocyclyl” refers to a nonaromatic monocyclic, bicyclic, tricyclic or tetracyclic ring system having at least one heteroatom selected from O, N, or S. Any ring atom capable of substitution can be substituted (e.g., by one or more substituents). Examples of heterocyclyl groups include, but are not limited to, tetrahydrofuranyl, tetrahydropyranyl, piperidinyl, morpholino, pyrrolinyl, pyrimidinyl, pyrrolidinyl and phenothiazinyl.

As used herein, a “heterocyclylalkyl” group refers to an alkyl moiety in which an alkyl hydrogen atom is replaced by a heterocyclyl group. Heterocyclylalkyl includes groups in which more than one hydrogen atom has been replaced by a heterocyclyl group.

As used herein, a “substituent” refers to a group “substituted” on an alkyl, alkenyl, alkynyl, aryl or heteroaryl group at any atom of that group that is capable of substitution. Suitable substituents include, without limitation, alkyl, alkenyl, alkynyl, cycloalkyl, haloalkyl (e.g., perfluoroalkyl such as CF₃), aryl, heteroaryl, arylalkyl, heteroarylalkyl, heterocyclyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkylamino, dialkylamino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, arylalkyl), S(O)ₙalkyl (where n is 0-2), S(O)ₙaryl (where n is 0-2), S(O)ₙheteroaryl (where n is 0-2), S(O)ₙheterocyclyl (where n is 0-2), amine (mono-, di-, alkyl, cycloalkyl, arylalkyl, heteroarylalkyl, aryl, heteroaryl, and combinations thereof), ester (alkyl, arylalkyl, heteroarylalkyl, aryl, heteroaryl), amide (mono-, di-, alkyl, arylalkyl, heteroarylalkyl, aryl, heteroaryl, and combinations thereof), sulfonamide (mono-, di-, alkyl, arylalkyl, heteroarylalkyl, and combinations thereof). Substituents on a group may be any one single substituent, or each independently any subset of the aforementioned substituents. A substituent may itself be substituted with any one of the above substituents.

### Ligands

In one aspect, the invention features a ligand of the following formula (I):

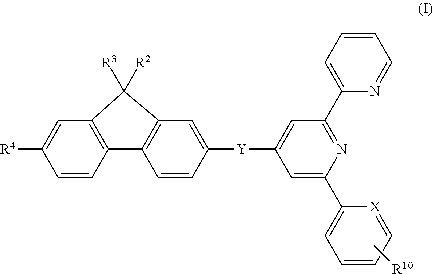

wherein:

R¹⁰ is H or —OR^{a};

R² and R³ are each independently C₁-C₂₄ alkyl, C₂-C₂₄ alkenyl, C₂-C₂₄ alkynyl or —(O—CH₂—CH₂)ₙ—OCH₃;

R⁴ is H, halo, aryl, heterocyclyl, arylalkyl, heterocyclylalkyl, arylalkynyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f};

X is C or N;

each n is independently an integer from 1-12;

each R^{a}, R^{b}, R^{c}, R^{d}, R^{e}, and R^{f} is independently selected from H, C₁-C₂₄ alkyl, C₂-C₂₄ alkenyl or C₂-C₂₄ alkynyl, aryl, heterocyclyl, arylalkyl, and heterocyclylalkyl; and

Y is a bond, —CH═CH—, —CH═CH-Ph-, or —C≡C—;

wherein when the ligand of formula (I) bears a charge, it further comprises one or more counterions.

In some embodiments, the ligand has one of the following formulae:

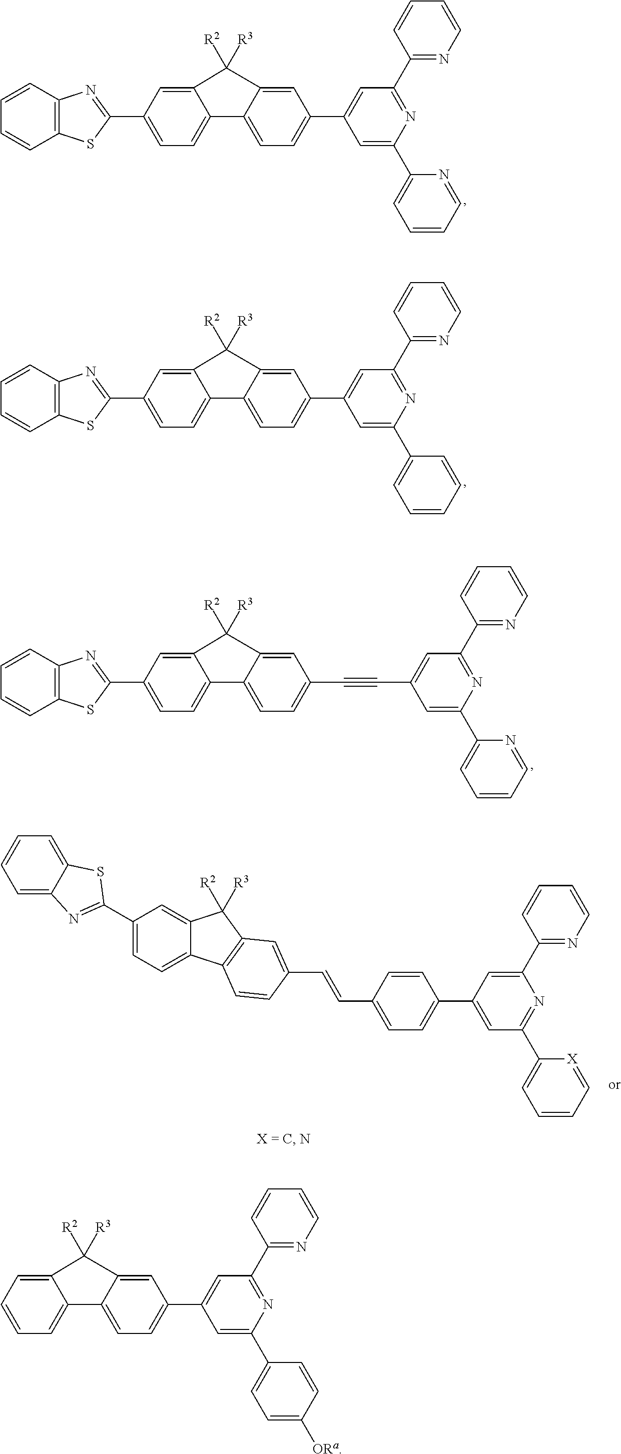

In another aspect, the invention features a ligand of the following formula (II):

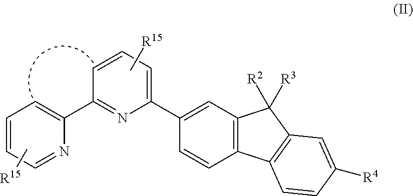

wherein:

the dashed line represents the presence or absence of an optionally substituted aromatic ring or fused aromatic rings;

R² and R³ are each independently C₁-C₂₄ alkyl, C₂-C₂₄ alkenyl, C₂-C₂₄ alkynyl or —(O—CH₂—CH₂)ₘ—OCH₃;

R⁴ is H, halo, aryl, heterocyclyl, arylalkynyl, arylalkyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl, or —CH═N—NH—R^{f};

each R¹⁵ is independently selected from H, halo, aryl, heterocyclyl, arylalkynyl, C₁-C₂₄ alkyl, C₂-C₂₄ alkenyl, or C₂-C₂₄ alkynyl;

each m is independently an integer from 1-12; and

each R^{b}, R^{c}, R^{d}, R^{e}, and R^{f} is independently selected from H, C₁-C₂₄ alkyl, C₂-C₂₄ alkenyl, C₂-C₂₄ alkynyl, heterocyclyl, arylalkyl, heterocyclylalkyl and aryl,

wherein when the ligand of formula (II) bears a charge, it further comprises one or more counterions.

In some embodiments, the ligand has one of the following formulae:

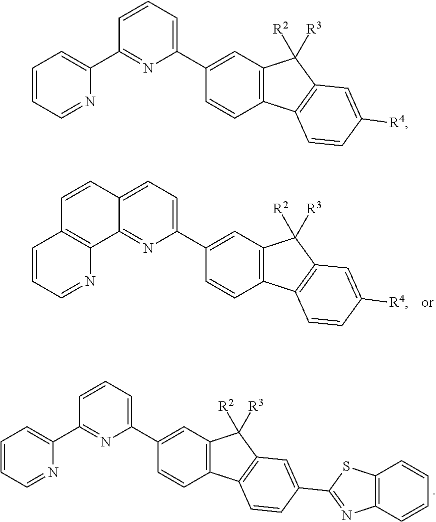

In another aspect, the invention features a ligand of formula (III):

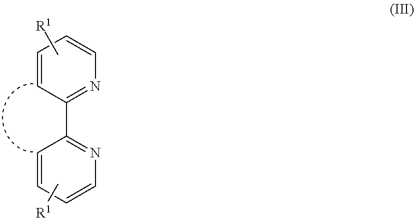

wherein:

each R¹ is independently a group of the following formula:

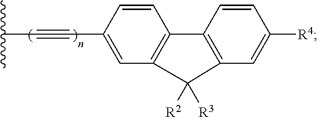

each n equals to 0 or 1;

each R² and R³ is independently C₁-C₂₄ alkyl, C₂-C₂₄ alkenyl or C₂-C₂₄ alkynyl, or —(O—CH₂—CH₂)ₘ—OCH₃;

each R⁴ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f}; and

each R^{b}, R^{c}, R^{d}, R^{e}, and R^{f} is independently selected from H, C₁-C₂₄ alkyl, C₂-C₂₄ alkenyl, C₂-C₂₄ alkynyl, heterocyclyl, arylalkyl, heterocyclylalkyl and aryl;

each m is independently an integer from 1-12; and

wherein the dashed line represents the presence or absence of an optionally substituted aromatic ring or fused aromatic rings; and

wherein when the ligand of formula (III) bears a charge, it further comprises one or more counterions.

In some embodiments, the ligand has one of the following formulae:

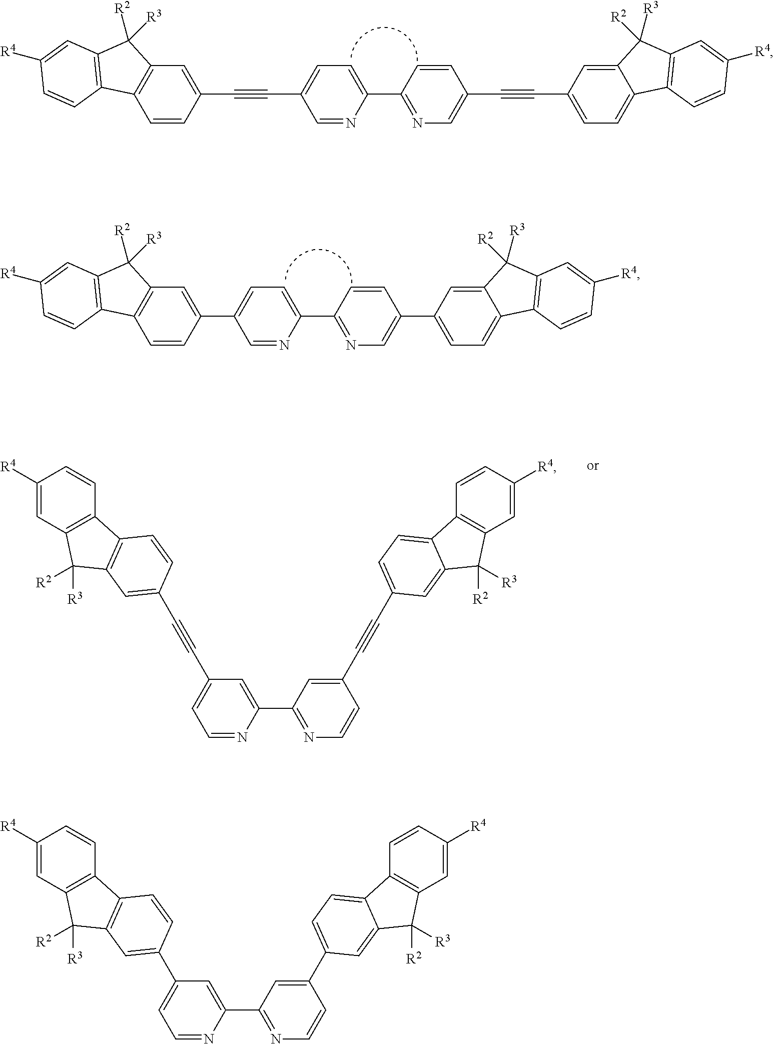

In Formulae (I), (II) and (III), R² and R³ are suitably a branched alkyl, such as

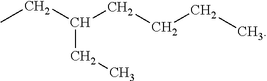

In certain embodiments, Y is suitably a bond or —C≡C— and X is
suitably C or N. In certain embodiments, R⁴ is

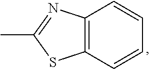

naphthalimide, naphthalene diimide, or —NR^{c}R^{d}. R^{c} and R^{d} are suitably alkyl, or aryl, such as phenyl.

### Metal Complexes

In one aspect, the invention features a metal complex of the following formula (IV):

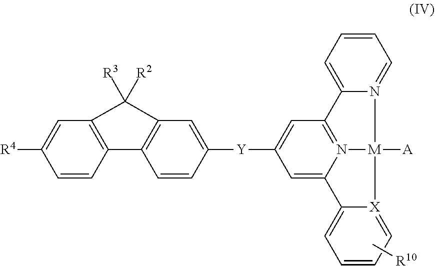

wherein:

R¹⁰ is H or —OR^{a};

R² and R³ are each independently C₁-C₂₄ alkyl, C₂-C₂₄ alkenyl, C₂-C₂₄ alkynyl or —(O—CH₂—CH₂)ₘ—OCH₃;

R⁴ is H, halo, aryl, heterocyclyl, arylalkyl, heterocyclylalkyl, arylalkynyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f};

A is selected from halo,

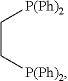

### and —C≡C—R^{g};

X=C or N;

each m is independently an integer from 1-12;

each R^{a}, R^{b}, R^{c}, R^{d}, R^{e}, R^{f}, and R^{g} is independently selected from H, C₁-C₂₄ alkyl, C₂-C₂₄ alkenyl or C₂-C₂₄ alkynyl, aryl, heterocyclyl, arylalkyl, arylalkynyl and heterocyclylalkyl; and

Y is a bond, —CH═CH—, —CH═CH-Ph-, or —C≡C—;

M is a metal ion, such as Pt²⁺, Pd²⁺, Ni²⁺, Zn²⁺, Cu²⁺, or Au³⁺; and

wherein when the complex of formula (IV) bears a charge, it further comprises one or more counterions.

In some embodiments, the complex has one of the following formulae:

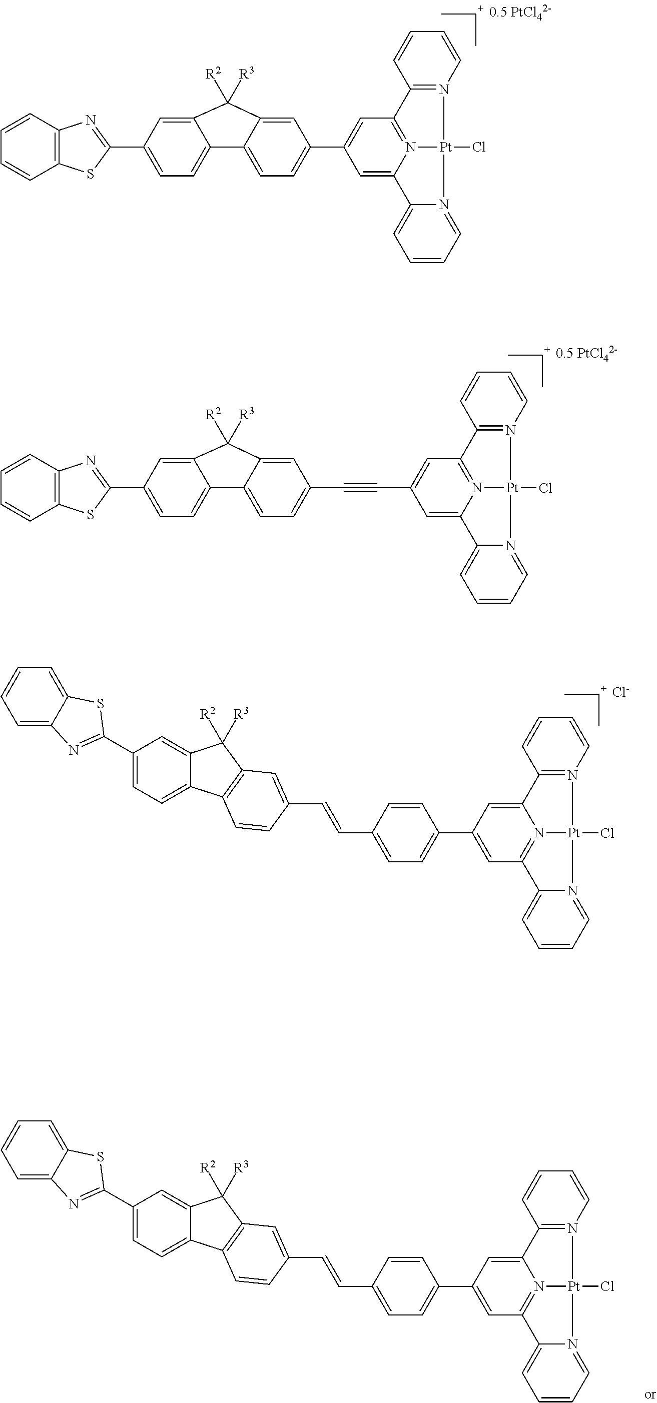

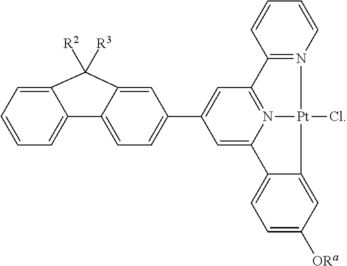

In another aspect, the invention features a metal complex of the following formula (V):

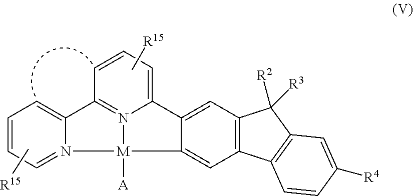

wherein:

the dashed line represents the presence or absence of an optionally substituted aromatic ring or fused aromatic rings;

R² and R³ are each independently C₁-C₂₄ alkyl, C₂-C₂₄ alkenyl, C₂-C₂₄ alkynyl or —(O—CH₂—CH₂)ₘ—OCH₃;

R⁴ is H, halo, aryl, heterocyclyl, arylalkynyl, arylalkyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl, or —CH═N—NH—R^{f};

R¹⁵ is each independently selected from H, halo, aryl, heterocyclyl, arylalkynyl, C₁-C₂₄ alkyl, C₂-C₂₄ alkenyl, or C₂-C₂₄ alkynyl;

A is selected from halo and —C≡C—R^{g};

M is a metal ion, such as Pt²⁺, Pd²⁺, Ni²⁺, Zn²⁺, Cu²⁺, or Au³⁺;

each m is independently an integer from 1-12; and

each R^{b}, R^{c}, R^{d}, R^{e}, R^{f}, and R^{g} is independently selected from H, C₁-C₂₄ alkyl, C₂-C₂₄ alkenyl, C₂-C₂₄ alkynyl, heterocyclyl, arylalkyl, heterocyclylalkyl, arylalkynyl and aryl,

wherein when the complex of formula (V) bears a charge, it further comprises one or more counterions.

In some embodiments, the compound has one of the following formulae:

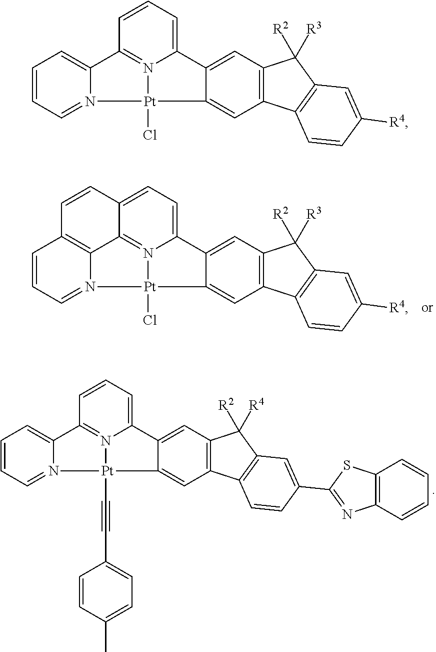

In another aspect, the invention features a metal complex of formula (VI):

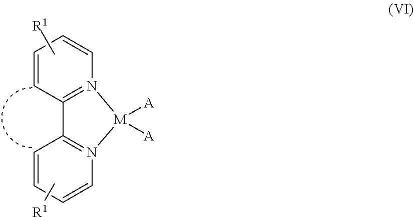

wherein:

the dashed line represents the presence or absence of an optionally substituted aromatic ring or fused aromatic rings;

each R¹ is independently a group of the following formula:

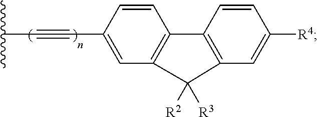

each n equals to 0 or 1

each R² and R³ is independently C₁-C₂₄ alkyl, C₂-C₂₄ alkenyl, C₂-C₂₄ alkynyl, or —(O—CH₂—CH₂)ₘ—OCH₃;

each m is independently an integer from 1-12;

each R⁴ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f}; and

each R^{b}, R^{c}, R^{d}, R^{e}, and R^{f} is independently selected from H, C₁-C₂₄ alkyl, C₂-C₂₄ alkenyl, C₂-C₂₄ alkynyl, heterocyclyl, arylalkyl, heterocyclylalkyl and aryl;

A is selected from halo and —C≡C—R^{g};

each R^{g} is independently selected from H, C₁-C₂₄ alkyl, C₂-C₂₄ alkenyl, C₂-C₂₄ alkynyl, heterocyclyl, arylalkyl, heterocyclylalkyl, arylalkynyl and aryl; and

M is a metal ion, such as Pt²⁺, Pd²⁺, Ni²⁺, Zn²⁺, Cu²⁺, or Au³⁺;

wherein when the complex of formula (VI) bears a charge, it further comprises one or more counterions.

In some embodiments, the complex has one of the following formulae:

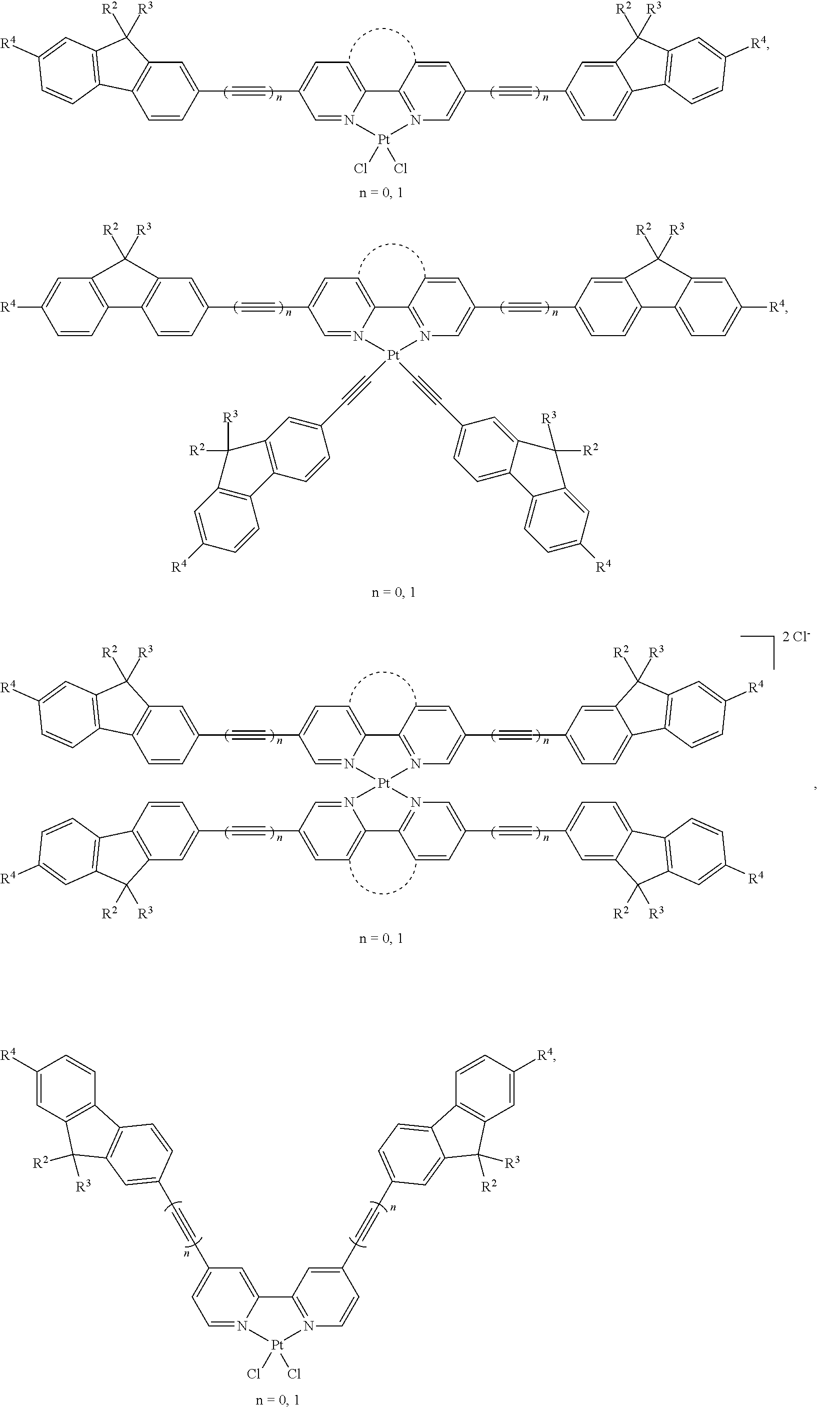

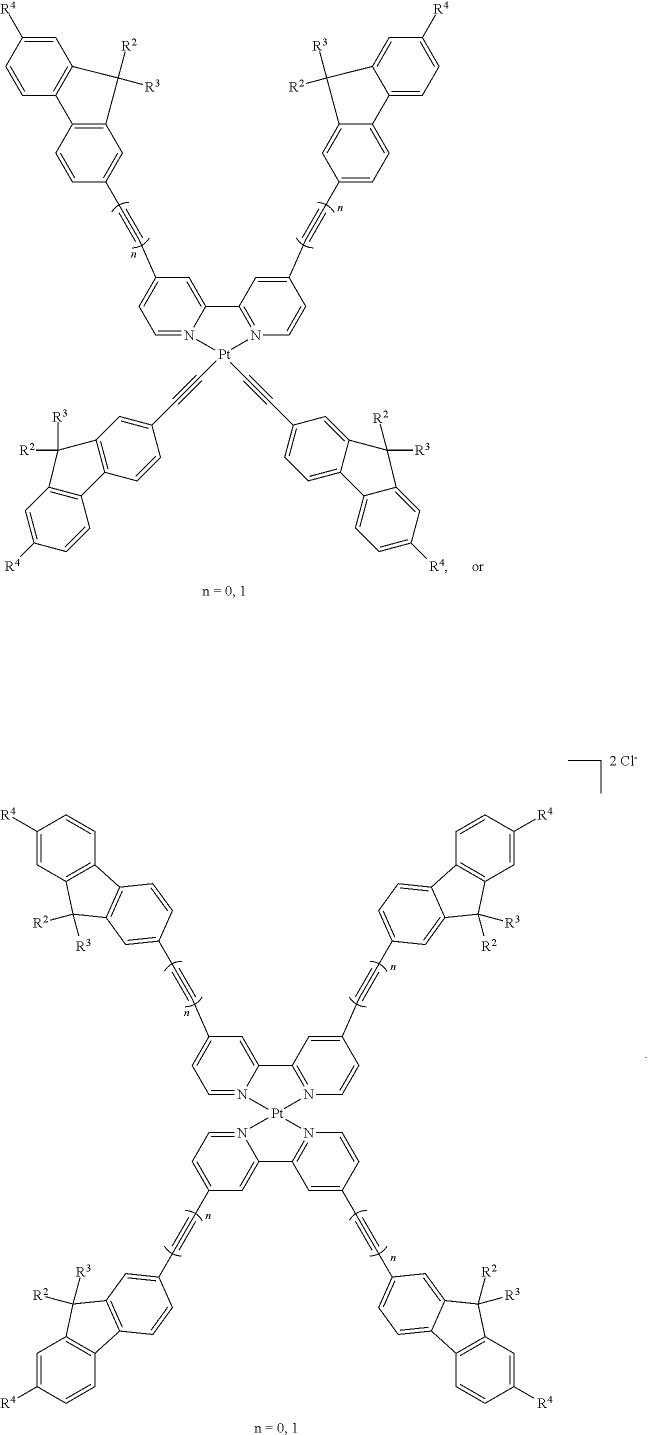

In another aspect, the invention features a metal complex of formula (VII):

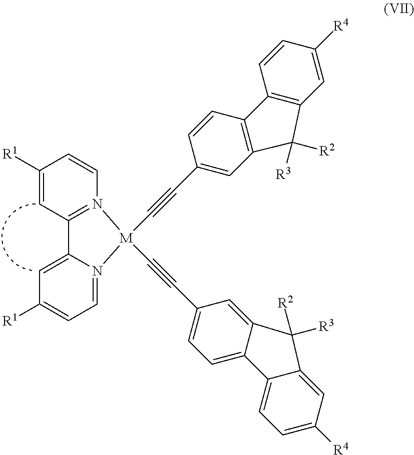

wherein

M is a metal ion, such as Pt²⁺, Pd²⁺, Ni²⁺, Zn²⁺, Cu²⁺, or Au³⁺;

the dashed line represents the presence or absence of an optionally substituted aromatic ring or fused aromatic rings;

each R¹ is independently C₁-C₂₄ alkyl, C₂-C₂₄ alkenyl, C₂-C₂₄ alkynyl, arylalkynyl or —(O—CH₂—CH₂)ₘ—OCH₃;

each R² and R³ is independently C₁-C₂₄ alkyl, C₂-C₂₄ alkenyl, C₂-C₂₄ alkynyl, or —(O—CH₂—CH₂)ₙ—OCH₃;

each R⁴ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f};

each m is independently an integer from 1-12;

each R^{b}, R^{c}, R^{d}, R^{e}, and R^{f} is independently selected from H, C₁-C₂₄ alkyl, C₂-C₂₄ alkenyl, C₂-C₂₄ alkynyl, arylalkyl, heterocyclylalkyl and aryl; and

wherein when the complex of formula (VII) bears a charge, it further comprises one or more counterions.

In some embodiments, the complex has the following formula:

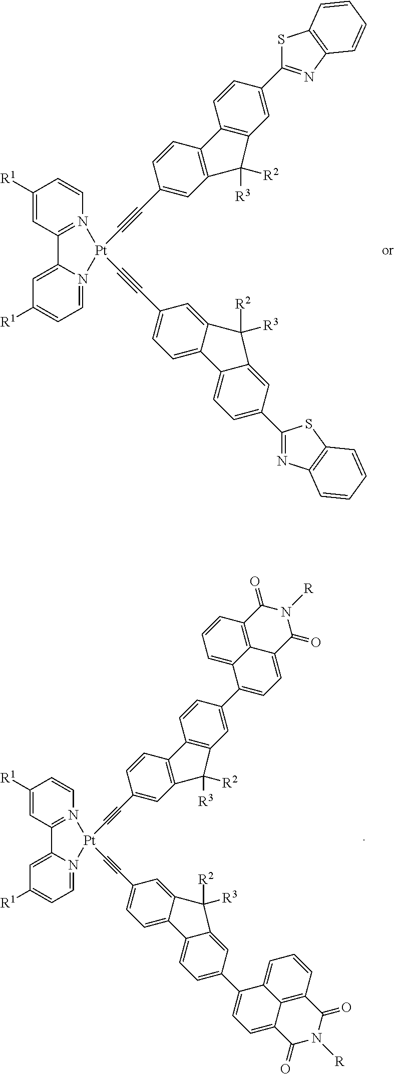

In another aspect, the invention features a metal complex of formula (VIII):

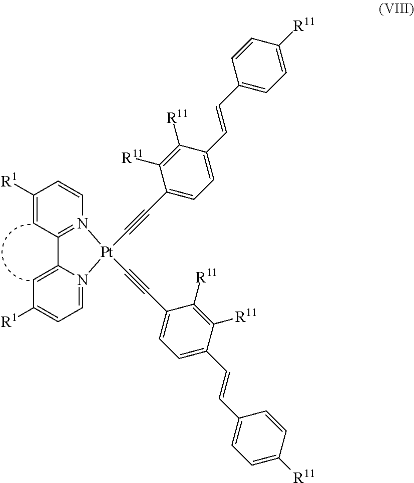

wherein

M is a metal ion, such as Pt²⁺, Pd²⁺, Ni²⁺, Zn²⁺, Cu²⁺, or Au³⁺;

the dashed line represents the presence or absence of an optionally substituted aromatic ring or fused aromatic rings;

each R¹ is independently C₁-C₂₄ alkyl, C₂-C₂₄ alkenyl, C₂-C₂₄ alkynyl, arylalkynyl or —(O—CH₂—CH₂)ₘ—OCH₃;

each R¹¹ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, and C₂-C₂₄ alkynyl;

each m is independently an integer from 1-12;

each R^{b}, R^{c}, R^{d}, and R^{e} is independently selected from H, C₁-C₂₄ alkyl, C₂-C₂₄ alkenyl, C₂-C₂₄ alkynyl, arylalkyl, heterocyclylalkyl and aryl, and

wherein when the complex of formula (VIII) bears a charge, it further comprises one or more counterions.

In Formulae (IV), (V), (VI), (VII) and (VIII) suitably, R¹ is independently a branched alkyl, such as

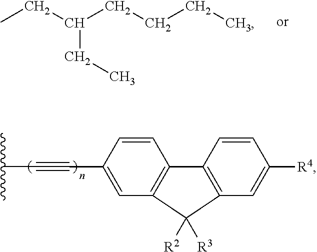

n equals to 0 or 1;
R² and R³ are independently a branched alkyl, such as

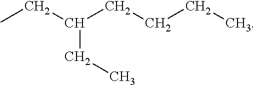

In certain embodiments, R¹¹ is Br, I, H, CHO, NO₂, OCH₃ or —NR^{c}R^{d}. A is Cl or

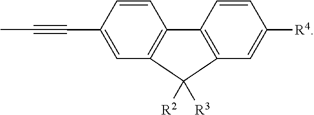

In certain embodiments, R⁴ is

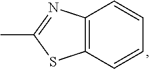

naphthalimide, naphthalene diimide, or —NR^{c}R^{d}. R^{c} and R^{d} are suitably alkyl or aryl, such as phenyl. Y is suitably a bond or —C≡C—. and X is suitably C or N. In certain embodiments, M is platinum.

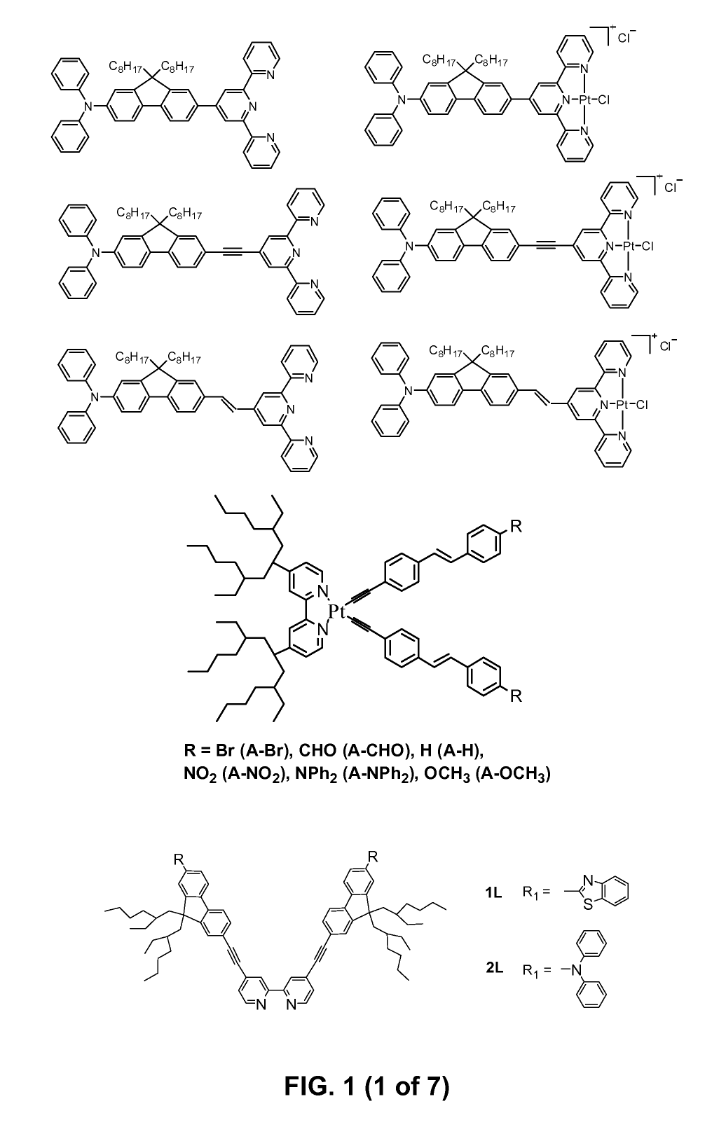

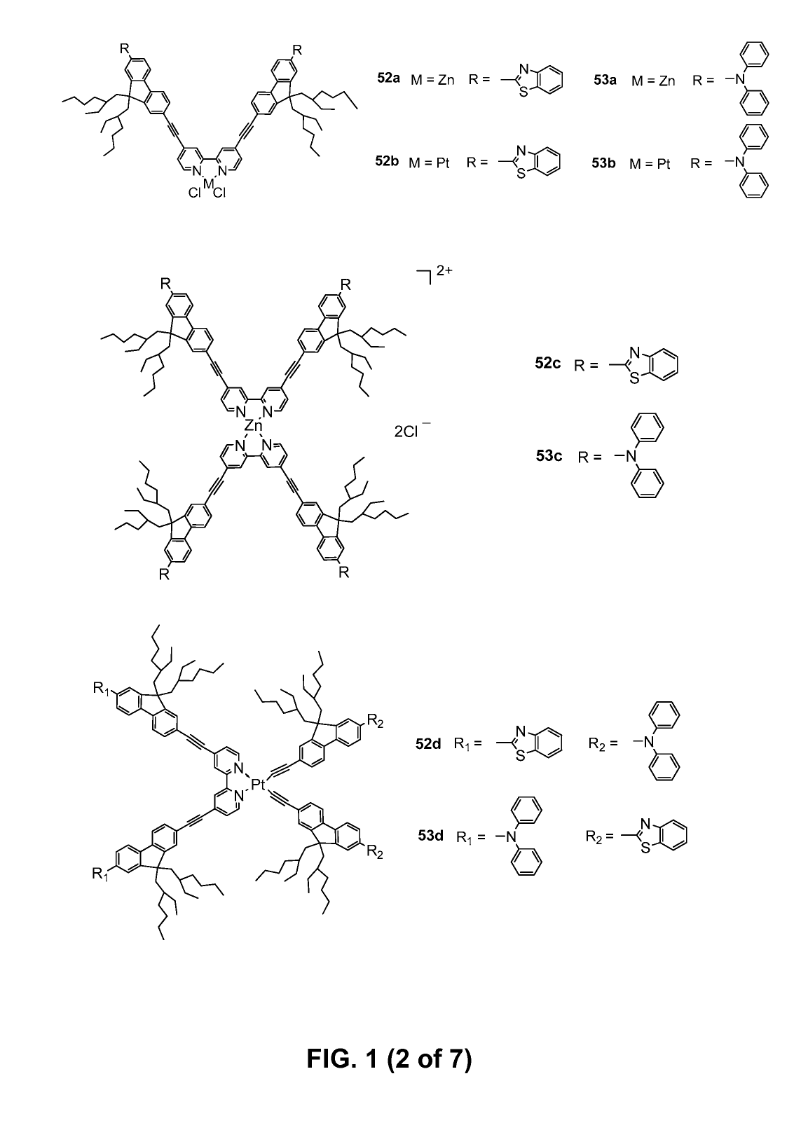

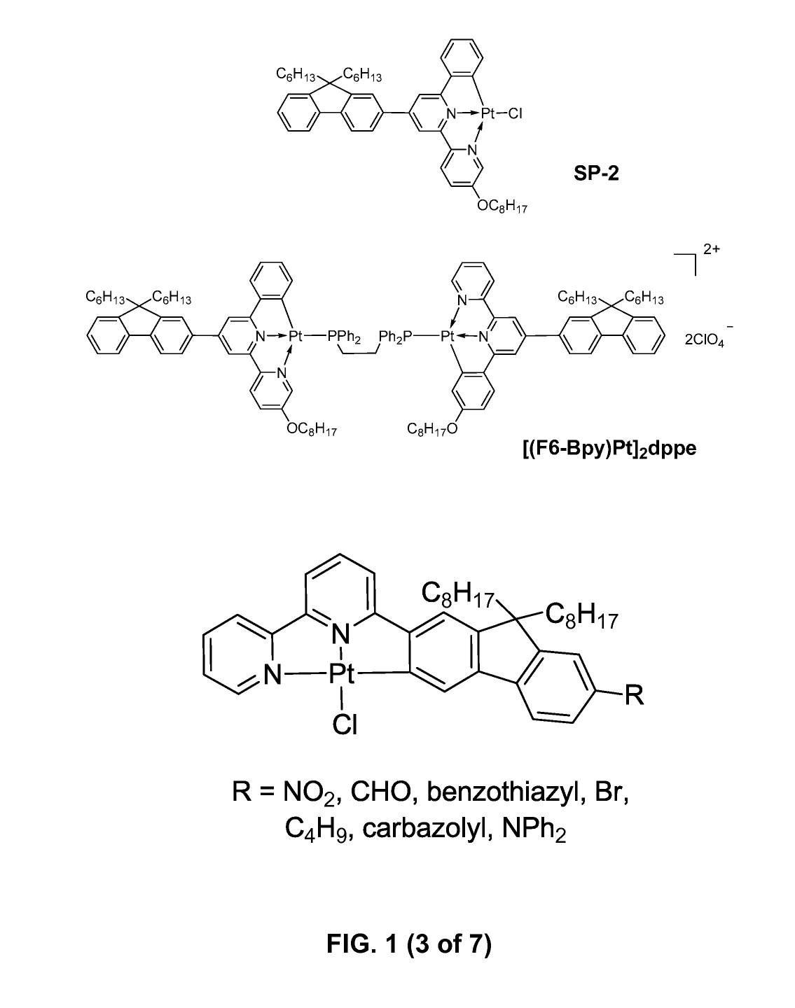

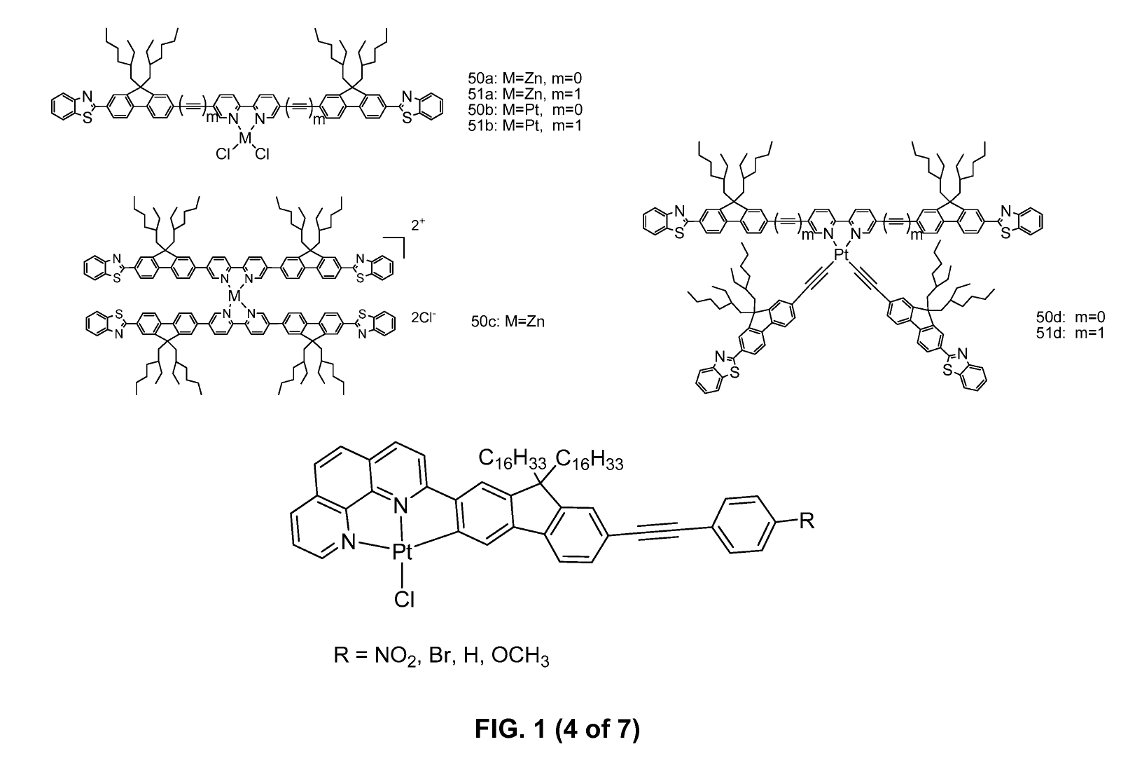

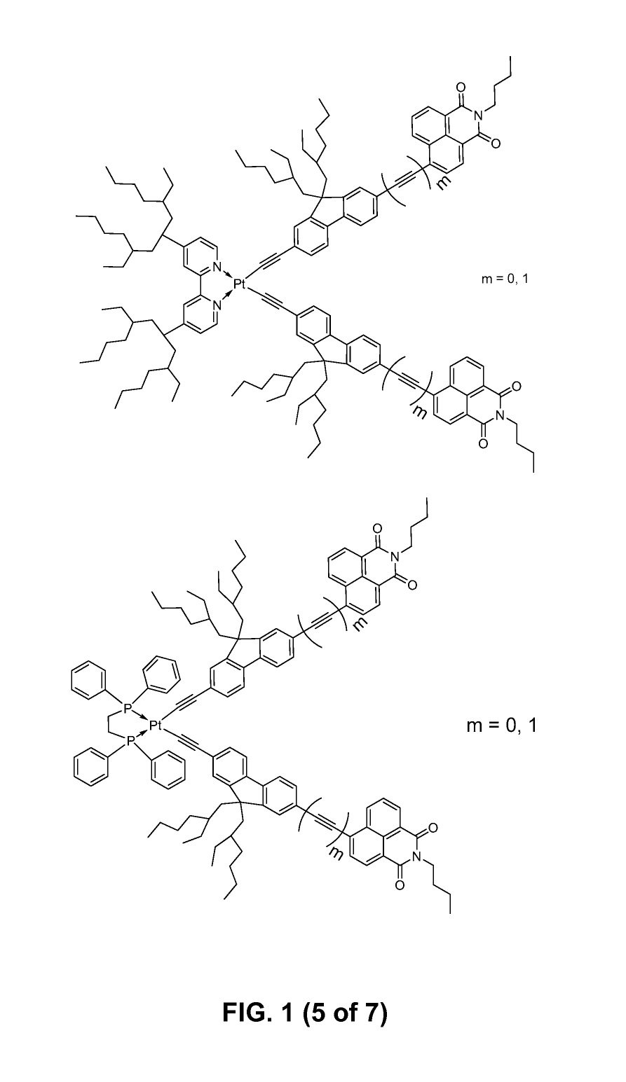

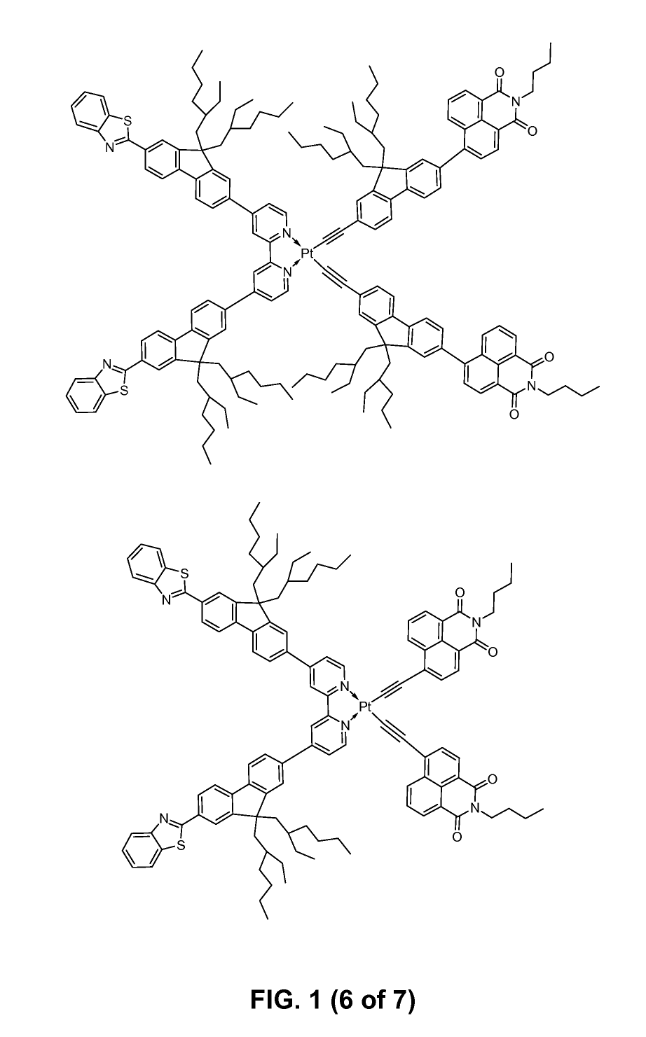

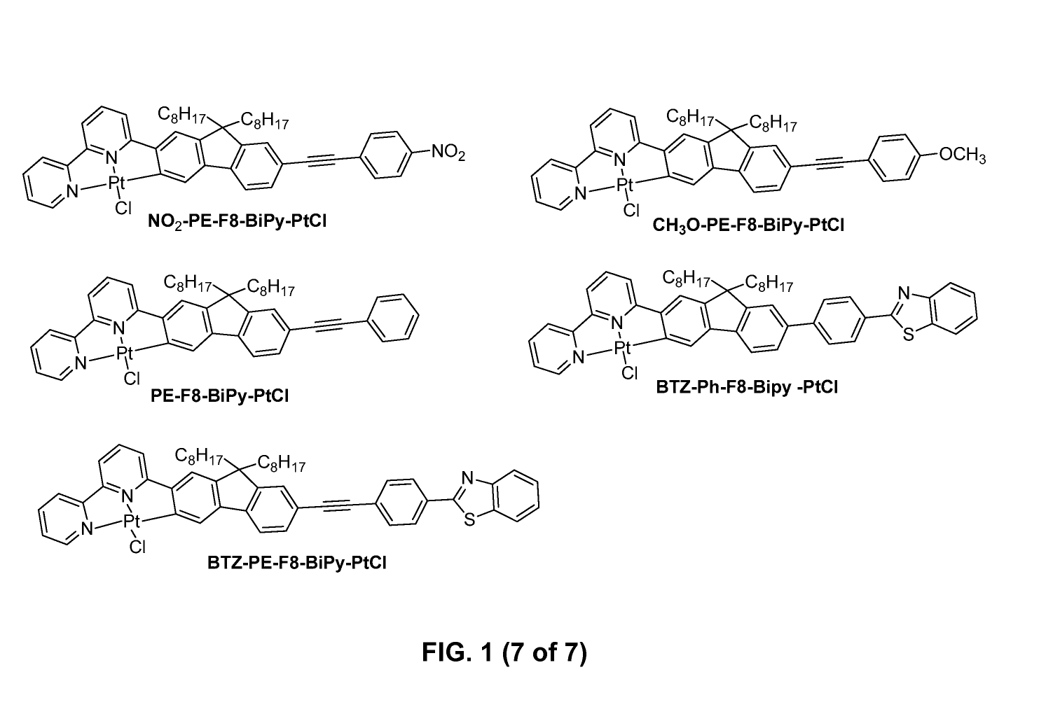

Suitable complexes and ligands according to the present invention include those shown in FIG. 1.

### General Synthetic Description

The ĈN̂N ligands with Y as a single bond in series I could be synthesized by using fluorene as the starting material. Bromonation and alkylation of fluorene, followed by reaction with BuLi at −78° C. yield the fluorenylaldehyde precusor, which reacts with 2-acetylpyridine to afford 1-(fluoren-2-yl)prop-2-en-1-one. Reaction of 1-(fluoren-2-yl)prop-2-en-1-one, N-(benzoylmethyl)-pyridinium bromide and NH₄OAc results in the formation of ĈN̂N ligands. Alternately, the ligands with Y as a single bond could be synthesized via Suzuki coupling reaction from 7-R⁴-fluoren-2-yl borate and 4-bromoterpyridine or 4-bromo-6-phenyl-2,2′-bipyridine. The ligands with Y as a double bond could be obtained by Heck reaction using 2-bromofluorene derivative and 4-vinylterpyridine or 4-vinyl-6-phenyl-2,2′-bipyridine as the starting materials. The ligands with Y as a triple bond could be synthesized via Sonogashira coupling reaction from a 2-ethynylfluorene derivative and 4-OTf-terpyridine or 4-OTf-6-phenyl-2,2′-bipyridine

The ligands II could be synthesized by Suzuki coupling reactions from the corresponding 7-R⁴-fluoren-2-yl borate and 6-bromo-2,2′-bipyridine; while 6-bromo-2,2′-bipyridine could be synthesized following literature procedures. Shin, D.; Switzer, C. Chem. Comm. 2007, 42, 4401. The fluorene borates could be synthesized by treating the corresponding 2-bromofluorene with BuLi in THF at −78° C. under argon, followed by addition of one equivalent isopropoxy-4,4,5,5-tetramethyl-1,3,2-dioxaborolane.

The ligands III with a triple bond connection between the substituted fluorene and the bipyridine could be synthesized by Sonogashira coupling reaction from the 4,4′-dibromo-2,2′-bipyridine or 5,5′-dibromo-2,2′-bipyridine and the 2-ethynyl-7-R⁴-fluorene. The ligands III with a single bond connection between the substituted fluorene and the bipyridine could be synthesized by Suzuki coupling reaction from the 4,4′-dibromo-2,2′-bipyridine or 5,5′-dibromo-2,2′-bipyridine and the 7-R⁴-fluoren-2yl borate.

The platinum(II) complexes IV-VIII could be synthesized using the corresponding terdentate (ĈN̂N or N̂N̂N) ligands or diimine ligands and K₂PtCl₄ or Pt(DMSO)₂Cl₂ salts to afford the platinum chloride complexes. In the case that the complexes bearing acetylide ligand(s), the corresponding platinum chloride complexes are used to react with the acetylide ligands in the presence of CuI as the catalyst and base.

### Uses of the Complexes and Ligands

The metal complexes and ligands according to the present invention may be used in many different end products. For example, the metal complexes may be used as ion sensors, such as for zinc, as organic vapor sensors, in optical-switching devices, and in organic light emitting diodes. For example, the ligands may be used as pH sensors, ion sensors, and in organic light emitting diodes.

The optical-switching device includes a pair of transparent substrates separated by a cavity therebetween. A nonlinear optical material including the synthesized metal complexes substantially fills the cavity between the substrates. If the incident light intensity is below a given level, the optical-switching device passes the incident light through. On the other hand, if the incident light intensity is above the given level, the optical-switching device does not pass the incident light through. Alternatively, one pump beam, which is at the wavelength of the absorption band maximum of the UV-vis absorption spectrum of the material, can be used to control the transmission of another beam (probe beam), which is not absorbed by the material via one-photon absorption. Without the pump beam, the probe beam will pass through; with the pump beam on, the probe beam does not pass through the device. Put another way, light controls light in the optical-switching devices.

In other embodiments, the synthesized metal complexes described above are used as light-emitting materials in an organic light-emitting diode (OLED). The OLED is constituted of an organic compound layer including the synthesized metal complexes, interposed between an anode and a cathode. When a DC voltage is applied to the OLED with the anode as a positive electrode and the cathode as a negative electrode, light is emitted.

In still other embodiments, the synthesized metal complexes described above are used as chemical sensors. The chemical sensor includes a fibrous substrate and a coating solution including a metal complex impregnated to the substrate. When contacted with organic vapors, the chemical sensor indicates the presence of the organic vapors by a vapochromic response, that is, by changing color. Mathew, I.; Sun, W. Dalton Trans. 2010, 39, 5885 (incorporated by reference herein). Non-limiting examples of the vapors that can be sensed by the disclosed chemical sensor include methanol, ethanol, iso-propanol, diethyl ether, acetonitrile, hexanes, acetone, benzene, dichloromethane, chloroform, and a combination thereof.

In still other embodiments, the synthesized metal complexes described above are used as ion sensors. The ion sensors include the dimethyl sulfoxide (DMSO) solution in which the ligands and metal complexes described herein are dissolved. When contacted with anions such as F⁻, H₂PO₄⁻ and OAc⁻, the color of the solutions can change; whereas addition of NO₃⁻, Cl⁻, Br⁻ and I⁻ (all as tetra-n-butylammonium salts, TBA salts) may have no effect on the solution color. B. Zhang, Y. Li, W. Sun, “Anion-Sensitive 2,4-Dinitrophenylhydrazone-Containing Terpyridine Derivative and Its Platinum Chloride Complex,” Eur. J. Inorg. Chem. (incorporated by reference herein).

### EXAMPLES

### Example 1

### Synthesis of Platinum(II) 6-Phenyl-4-(9,9-dihexylfluoren-2-yl)-2,2′-bipyridine Complexes with Phenothiazinyl Acetylide Ligand

All of the reagents and solvents were purchased from Alfa Aesar or Aldrich. Solvents were used as received unless otherwise stated. ¹H NMR spectra were measured on a Varian 400 MHz VNMR spectrometer. ESI-HRMS analyses were conducted on a Bruker Daltonics BioTOF III mass spectrometer. Elemental analyses were performed by NuMega Resonance Labs, Inc. in San Diego, Calif.

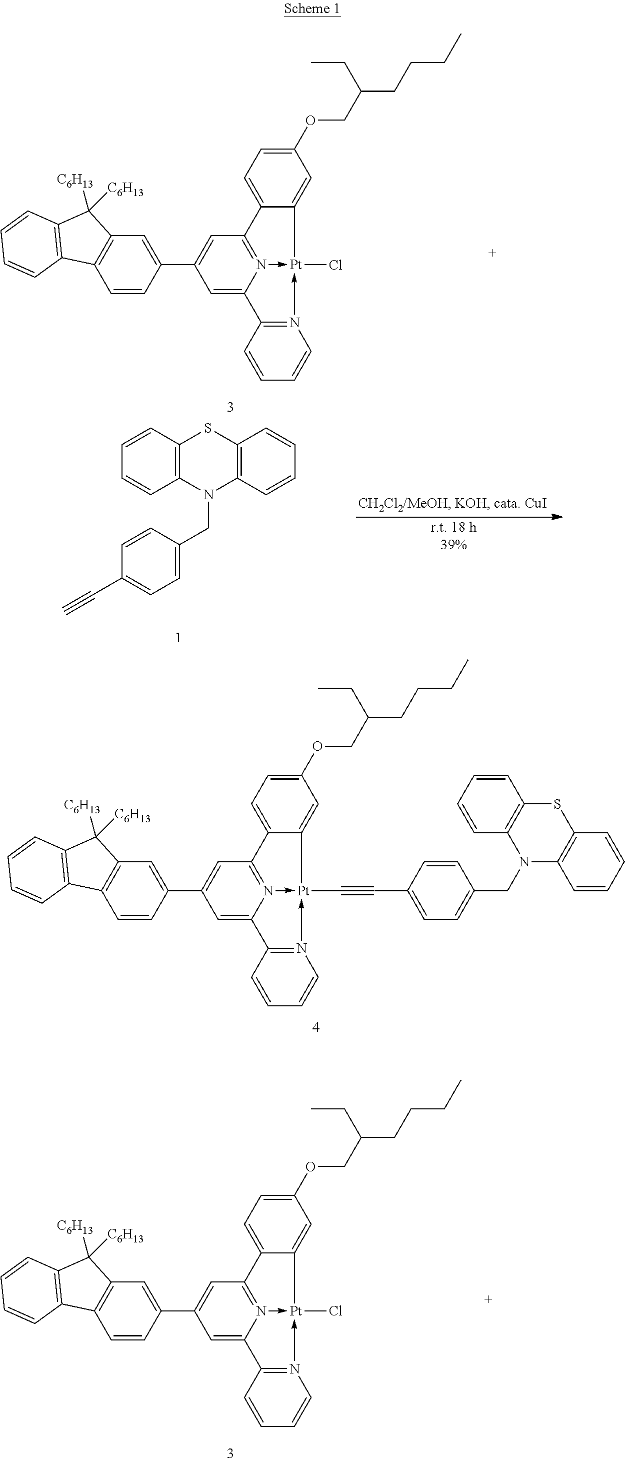

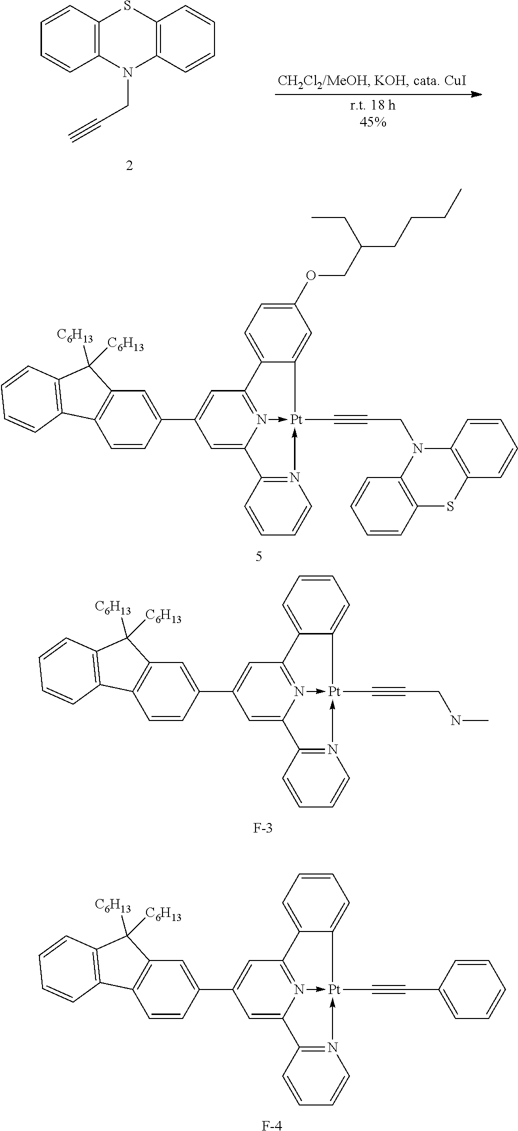

Compounds 1 and 2 were synthesized according to the reported procedures. McGarrah, J. E.; Kim, Y. J.; Hissler, M.; Eisenberg, supra; Sae-Lim, C.; Sandman, D. J.; Foxman, B. M.; Sukwattanasinitt, A. M. J. Macromolecular Sci., Part A: Pure Appl. Chem. 2006, 43, 1929 (incorporated by reference herein). The synthesis of complex 3 was reported previously. Shao, P.; Li, Y.; Yi, J.; Pritchett, T. M; Sun, W., Inorg. Chem. 2010, 49, 4507-4517 (incorporated herein by reference).

Complex 4: Complex 3 (0.18 g, 0.20 mmol), compound 1 (0.08 g, 0.25 mmol), KOH (0.02 g, 0.36 mmol), and catalytic amount of CuI were dissolved in a mixed solvent of CH₂Cl₂ and CH₃OH (v/v=50 mL/30 mL). The reaction mixture was stirred at room temperature for 18 hrs. After removal of the solvent, the residue was dissolved in CH₂Cl₂, and then washed with brine three times to remove KOH. The CH₂Cl₂ layer was combined and dried over Na₂SO₄. Solvent was then removed and the crude product was purified by chromatography on a silica gel column using CH₂Cl₂ as the eluent. The product was recrystallized from CH₂Cl₂/ethanol to yield 98 mg dark red solid (yield: 39%). ¹H NMR (400 MHz, CDCl₃, 25° C., TMS): δ 9.26 (d, 1H, J=4.8 Hz), 8.03 (t, 1H, J=7.6 Hz), 7.97 (d, 1H, J=8.4 Hz), 7.80 (d, 1H, J=8.0 Hz), 7.74 (t, 1H, J=2.8 Hz), 7.67 (t, 2H, J=3.8 Hz), 7.60 (d, 2H, J=6.4 Hz), 7.57 (d, 1H, J=2.8 Hz), 7.51 (d, 3H, J=7.2 Hz), 7.40 (d, 1H, J=7.6 Hz), 7.35 (d, 3H, J=3.2 Hz), 7.18 (d, 2H, J=7.6 Hz), 7.06 (dt, 2H, J=7.6 Hz and 1.2 Hz), 6.97 (tt, 2H, J=7.2 Hz and 1.2 Hz), 6.84 (t, 2H, J=7.6 Hz), 6.67 (d, 2H, J=8.0 Hz), 6.61 (dd, 1H, J=7.6 Hz and 1.2 Hz), 5.06 (s, 2H), 3.93 (d, 2H, J=5.6 Hz), 2.02 (t, 4H, J=8 Hz), 1.72 (m, 1H), 1.51 (d, 1H, J=1.6 Hz), 1.50-1.37 (m, 4H), 1.29-1.26 (m, 5H), 1.31-1.05 (m, 5H), 1.31-1.05 (m, 12H), 0.89-0.83 (m, 6H), 0.75 (t, 5H, J=7.2 Hz), 0.65 (s, 3H). ESI-HRMS: m/z calcd for [C₇₀H₇₃N₃OPtS+Na]⁺: 1222.5034; found, 1222.5048. Anal. Calcd (%) for C₇₀H₇₃N₃OPtS.CH₃OH: C, 69.16; H, 6.74; N, 3.46; found: C, 69.24, H, 6.30, N, 3.41.

Complex 5: Complex 3 (0.18 g, 0.20 mmol), compound 2 (0.11 g, 0.30 mmol), KOH (0.02 g, 0.36 mmol), and catalytic amount of CuI were dissolved in a mixed solvent of CH₂Cl₂ and CH₃OH (v/v=50 mL/30 mL). The reaction mixture was stirred at room temperature for 18 hrs. After removal of the solvent, the residue was dissolved in CH₂Cl₂, and then washed with brine three times to remove KOH. The CH₂Cl₂ layer was combined and dried over Na₂SO₄. Solvent was then removed and the crude product was purified by chromatography on a silica gel column using CH₂Cl₂ as the eluent. The product was recrystallized from CH₂Cl₂/ethanol to yield 102 mg dark red solid (yield: 46%). ¹H NMR (400 MHz, CDCl₃, 25° C., TMS): δ 8.58 (d, 1H, J=4.8 Hz), 7.74 (d, 2H, J=8.4 Hz), 7.67 (d, 2H, J=7.6 Hz), 7.58 (t, 2H, J=3.8 Hz), 7.55 (s, 1H), 7.84 (d, 2H, J=8.4 Hz), 7.33-7.29 (m, 4H), 7.14-7.05 (m, 6H), 6.90 (dd, 1H, J=8.8 Hz and 4.8 Hz), 6.83 (t, 2H, J=7.6 Hz), 6.36 (dd, 1H, J=8.4 Hz and 2.4 Hz), 4.84 (s, 2H), 3.43 (d, 2H, J=5.2 Hz), 1.97 (t, 4H, J=8.0 Hz), 1.47-1.43 (m, 1H), 1.36-1.16 (m, 10H), 1.09-1.00 (m, 12H), 0.84-0.75 (m, 6H), 0.69 (t, 5H, J=6.8 Hz), 0.62 (s, 3H). ESI-HRMS: m/z calcd for [C₆₄H₆₉N₃OPtS+Na]⁺, 1145.4706; found, 1145.4662. Anal. Calcd (%) for C₆₄H₆₉N₃OPtS.CH₃OH: C, 67.15; H, 6.59; N, 3.54; found: C, 67.57; H, 6.37; N, 3.64.

### Example 2

### Photophysics of Platinum(II) 6-Phenyl-4-(9,9-dihexylfluoren-2-yl)-2,2′-bipyridine Complexes with Phenothiazinyl Acetylide Ligand

Two platinum 6-phenyl-4-(9,9-dihexylfluoren-2-yl)-2,2′-bipyridine complexes (4 and 5) with phenothiazinyl (PTZ) acetylide ligand were characterized. Their UV-vis absorption and emission characteristics in solution and in LB film were systematically investigated. The triplet transient difference absorption and nonlinear absorption properties were also studied for these complexes. Both complexes exhibit a broad metal-to-ligand charge transfer/intraligand charge transfer/ligand-to-ligand charge transfer (¹MLCT/¹ILCT/¹LLCT) absorption band between 400 and 500 nm and a ³MLCT/³ILCT/³π,π* emission band at ˜594 nm at room temperature, which blue shifts at 77 K. Both UV-vis absorption and emission spectra show negative solvatochromic effect. The triplet excited-state lifetime at room temperature for complex 4 is ca. 1.2 μs, which is longer than that for complex 5 (˜600 ns). The emission quantum yield of complex 4 in toluene is 0.18, while it is 0.053 for complex 5. Both of the complexes also exhibit broad and moderately strong triplet transient absorption from the near-UV to the near-IR spectral region. However, 5 exhibits stronger reverse saturable absorption than complex 4 does at 532 nm for ns laser pulses. This is attributed to the weaker ground-state absorption but stronger triplet excited-state absorption at 532 nm for 5 than for 4, which leads to a larger ratio of excited-state absorption cross-section to ground-state absorption for 5 than 4. In addition, LB films of 4 and 5 were prepared and characterized by AFM technique. The UV-vis absorption and emission spectra of the LB films of 4 and 5 were also investigated and compared with those obtained in solution.

### Photophysical Measurements

The UV-vis absorption spectra were measured on an Agilent 8453 spectrophotometer in a 1-cm quartz cuvette in HPLC-grade solvent. The steady state emission spectra were obtained using a SPEX fluorolog-3 fluorometer/phosphorometer. The emission quantum yields were measured by the comparative method (Demas, J. N.; Crosby, G. A. J. Phys. Chem. 1971, 75, 991) (incorporated by reference herein) in degassed toluene solution. A degassed [Ru(bpy)₃]Cl₂ in aqueous solution (Φₑₘ=0.042, λₑₓ=436 nm) (Van Houten, J.; Watts, R. J. Am. Chem. Soc. 1976, 98, 4853) (incorporated by reference herein) was used as the reference. The excited-state lifetimes and the triplet transient difference absorption spectra were obtained in toluene solutions on an Edinburgh LP920 laser flash photolysis spectrometer. The third harmonic output (355 nm) of a Nd:YAG laser (Quantel Brilliant, pulse width (fwhm)=4.1 ns, the repetition rate was set to 1 Hz) was used as the excitation source. Each sample was purged with Ar for 30 min prior to each measurement. The triplet excited-state absorption coefficients were measured using partial saturation method. Carmichael, I.; Hug, G. L. J. Phys. Chem. Ref Data 1986, 15, 1 (incorporated by reference herein).

### Nonlinear Transmission Measurement.

The experimental setup was described previously. Sun, W.; Zhu, H.-J.; Barron, P. Chem. Mater. 2006, 18, 2602 (incorporated by reference herein). The second harmonic (λ=532 nm) of a 4.1 ns (fwhm), 10 Hz, Q-switched Quantel Brilliant Nd:YAG laser was used as the light source. The laser beam was focused by a f=30 cm plano-convex lens to the center of a 2 mm thick quartz cuvette that contained the sample solution. The radius of the beam waist was approximately 75 μm. Two Molectron J4-09 pyroelectric probes and an EPM2000 energy/power meter were used to monitor the incident and output energies.

### LB Film Preparation

Surface pressure-mean molecular area isotherm measurement and LB film preparation were carried out on a KSV minitrough (Teflon coating, 7.5×30×1 cm³). The trough and the barriers were cleaned using ethanol and ultra pure water with a resistance of 18 MΩ·cm. Both 4 and 5 were dissolved in HPLC-grade CHCl₃ solvent for the surface pressure—mean molecular area measurement (c=1.16×10⁻³ mol/mL for 4 and 0.78 mmol/mL for 5). 50 μl of solution was spread on ultra pure water subphase at 25±1° C. and was left for 25 mins. to allow for CHCl₃ to evaporate. The compression rate was kept at 5 mm/min The isotherm measurement for each sample was repeated at least twice.

The LB films of 4 and 5 were deposited on hydrophilic glass slides using a dipping method with the transfer ratio in a range of 0.6-1. The glass slides were cleaned using detergent and water first, followed by soaking in concentrated H₂SO₄ for at least 1 hour. This would make the surfaces hydrophilic before the deposition. The slides were then cleaned by ultra pure water.

Surface morphology of the films prepared was studied by AFM technique using the tapping mode of a Veeco DI-3100 with a silicon nitride probe. Each sample was repeated three times to verify that the observed AFM images are not from the defective surface.

### Results

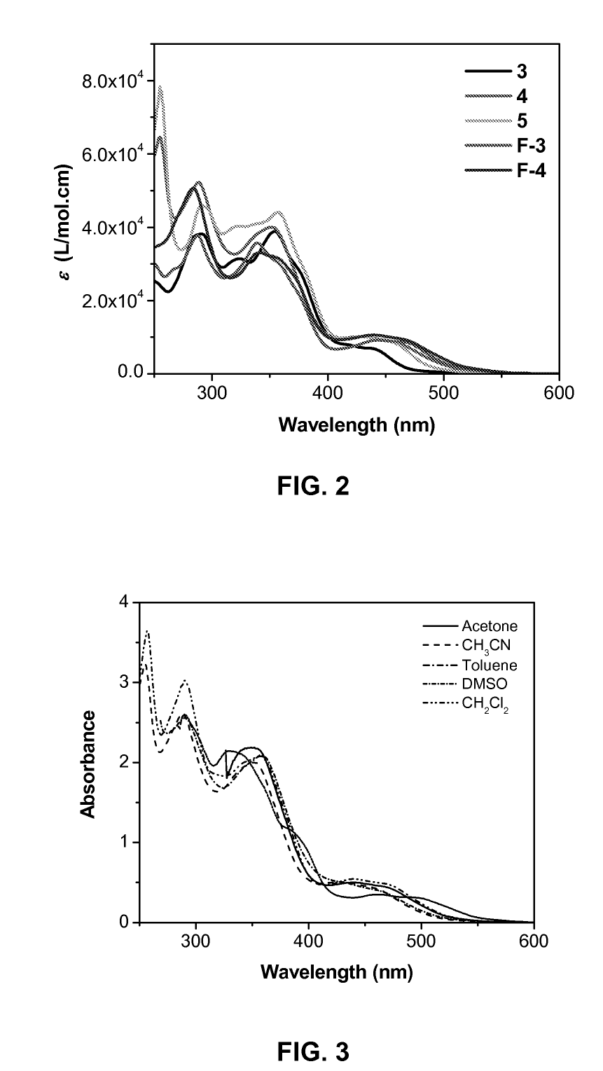

The electronic absorption spectra of 4 and 5 in dichloromethane solution are shown in FIG. 2, and the absorption band maxima and molar extinction coefficients are listed in Table 1. For comparison purpose, the UV-vis absorption spectra and corresponding data for their platinum chloride precursor 3 and their platinum acetylide analogues without the PTZ unit (F-3 and F-4) are also included in FIG. 2 and Table 1. The absorptions for both 4 and 5 follow Beer-Lambert's law in the concentration range of 10⁻⁶-10⁻⁴ mol/L, illustrating that no aggregation occurs in this concentration range. Both complexes exhibit intense high-energy absorption bands at 250-390 nm, which are attributed to the ¹π,π* transitions within the fluorenyl substituted ĈN̂N ligand and the BTZ acetylide ligand. The broad low-energy band in the range of 400-500 nm can be tentatively assigned as the ¹MLCT/¹ILCT/¹LLCT (the ILCT refers to the charge transfer from the fluorenyl substituent to the bipyridine component or from the phenyl ring to the bipyridine component within the fluorenyl ĈN̂N ligand) transitions with reference to the similar band in other platinum terpyridyl or ĈN̂N acetylide complexes. Yang, Q. Z.; Wu, L. Z.; Wu, Z. X.; Zhang, L. P.; Tung, C. H. Inorg. Chem. 2002, 41, 5653; Liu, X.-J.; Feng, J.-K.; Meng, J.; Pan Q.-J.; Ren, A.-M.; Zhou, X.; Zhang, H.-X. Eur. J. Inorg. Chem. 2005, 1856 (incorporated by reference herein). The ¹MLCT/¹ILCT/¹LLCT band in 4 is slightly broader than that in 5.

The charge transfer nature of the low-energy absorption band is supported by the negative solvatochromic effect for these two complexes in different polarity solvents (demonstrated in FIG. 3). Low-polarity solvents, such as toluene and dichloromethane, cause a pronounced bathochromic shift for the low-energy absorption band in comparison to that in more polar solvents, such as CH₃CN. This indicates that the ground states of both complexes are more polar than that of the excited states, which is a characteristic of a charge-transfer transition.

TABLE 1
Photophysical parameters of 3, 4 and 5.

| | λ_{abs}/nm | | λₑₘ/nm | λ_{T1-Tn}/nm |
|---|---|---|---|---|
| | (ε/L · mol⁻¹ · | λₑₘ/nm | (τ/μs)^{f} | (ε_{T1-Tn}/L · mol⁻¹ · |
| | cm⁻¹)^{c} | (τ/ns; Φₑₘ) R.T. | 77 K | cm⁻¹; τ/ns) |
| 3^{a} | 439 (6800), | 591 (950; 0.047)^{d} | 542 (16.0), | 475 (5500; 620), |
| | 419 (7700), | | 586 (15.5) | 665 (4820; 680), |
| | 354 (38100), | | | 800 (—; 480)^{g} |
| | 323 (30800), | | | |
| | 291 (37500) | | | |
| 4^{b} | 462 (8280), | 594 (1210; 0.18)^{e} | 546 (10.2), | 420 (12810; |
| | 428 (10180), | | 589 (10.6) | 1260), 636 |
| | 352 (40020), | | | (8190; 1210)^{h} |
| | 288 (52220), | | | |
| | 255 (64540) | | | |
| 5^{b} | 451 (8980), | 597 (600; 0.053)^{e} | 546 (9.7), | 415 (9560; 560), |
| | 427 (10180), | | 590 (9.3) | 505 (4830; 630)^{h} |
| | 359 (43840), | | | |
| | 292 (45840), | | | |
| | 255 (78280) | | | |
| F-3^{a} | 463 (8800), | 593 (680; 0.073)^{d} | 550 (14.0), | 400 (11480; 670), |
| | 443 (9200), | | 590 (14.2) | 635 (3790; 660)^{g} |
| | 355 (30800), | | | |
| | 339 (35700), | | | |
| | 288 (37700) | | | |
| F-4^{a} | 465 (9200), | 593(980; 0.076)^{d} | 554 (14.0), | 400 (11000; 880), |
| | 441 (10300), | | 584 (14.5) | 645 (1670; 800)^{g} |
| | 355 (30900), | | | |
| | 341 (31900), | | | |
| | 284 (48200) | | | |

^{a}From Shao, P.; Li, Y.; Yi, J.; Pritchett, T. M. W. Sun, supra.
^{b}This Example.
^{c}In CH₂Cl₂.
^{d}In CH₂Cl₂ at a concentration of 5 × 10⁻⁵ mol/L.
^{e}Measured at a toluene solution with A = 0.1 at 436 nm.
^{f}In butyronitrile glassy solution at 77 K. c = 5 × 10⁻⁵ mol/L for 3, F-3 and F-4, and 3.5 × 10⁻⁵ mol/L for 4 and 5.
^{g}In CH₃CN.
^{h}In toluene.

In comparison to the UV-vis absorption spectrum of the corresponding platinum chloride complex 3, the low-energy absorption band of 4 and 5 is 23 nm and 17 nm red-shifted, respectively. This is the result of the electron donating PTZ acetylide ligand, which not only causes the red-shift of the ¹MLCT band due to the reduced energy gap between the destabilized platinum based HOMO and the bipyridine based LUMO, but also admixes ¹LLCT character into the lowest excited state. In addition, the molar extinction coefficients of 4 and 5 are somewhat larger than those of 3, reflecting the influence of the electron-donating acetylide ligand. In contrast, introducing the PTZ substituent on the acetylide ligand exhibits minor effect on the low-energy charge transfer bands of 4 and 5 in comparison to those in their acetylide analogues F-3 and F-4. However, the molar extinction coefficients of the UV absorption bands in 4 and 5 are larger than those in F-3 and F-4.

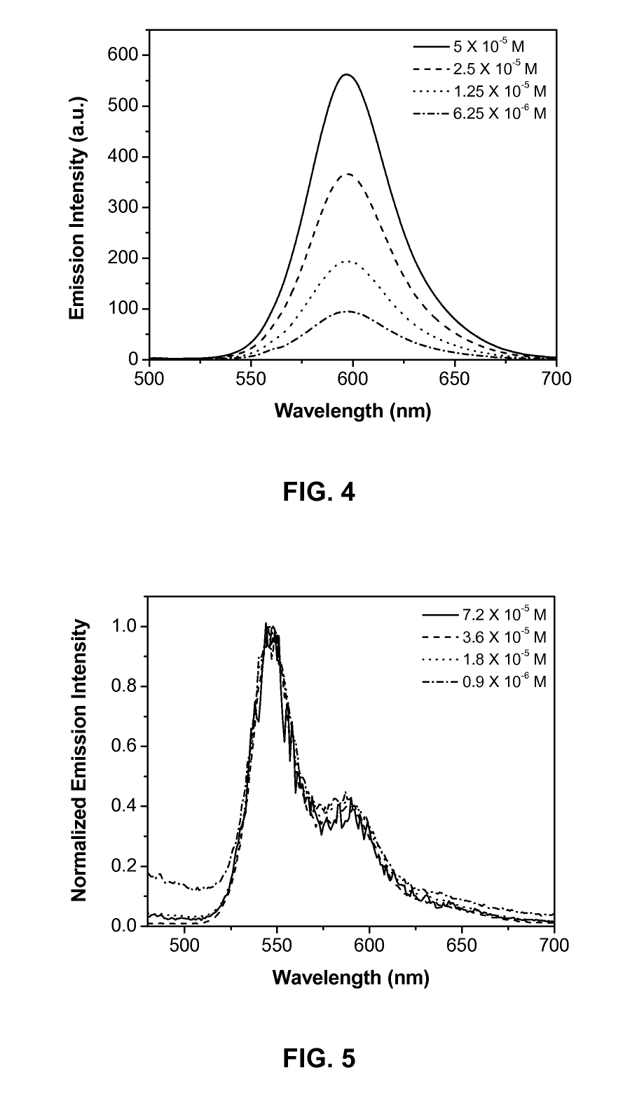

Complexes 4 and 5 are emissive at room temperature. As shown in FIG. 4, when the concentration of the solution increases from 6.25×10⁻⁶ to 5.0×10⁻⁵ mol/L, the intensity of the emission band at ˜590 nm keeps increasing; while the lifetime of 4 and 5 at different concentrations are essentially the same. This implies that no self-quenching occurs in the concentration range studied. The lifetimes of 4 and 5 are approximately 1.2 μs and 600 ns, respectively. Considering the long lifetimes and the large Stokes shifts for both complexes, it is reasonable to believe that the emission originates from a triplet excited state, likely from the ³MLCT/³ILCT/³π,π* state (the ILCT and π,π* states refer to the charge transfer and π,π* states within the fluorenyl ĈN̂N ligand) with reference to the nature of the emission from related platinum complexes with alkoxyl substituent. The involvement of the ³ILCT/³π,π* character can be partially supported by the fact that the emission energies of these two complexes are essentially the same, and they are similar to that of their corresponding precursor 3. This similarity indicates that the acetylide ligand has negligible effect on the emission energy, thus the emitting state should primarily be related to the fluorenyl ĈN̂N platinum component. This phenomenon is similar to that of the platinum ĈN̂N complexes with alkoxyl substituent.

Another piece of evidence that supports the admixture of the ³ILCT/³π,π* character into the emitting state arises from the minor solvent effect on the emission energy, as listed in Table 2 for 4 and 5. This reflects the delocalization of the emitting state and is consistent with that observed in the platinum ĈN̂N complexes with alkoxyl substituent. Although the solvatochromic effect on the emission energy is insignificant, it is still evident that less polar solvents, such as CH₂Cl₂, cause a slight bathochromic shift of the emission band compared to polar solvents, such as CH₃CN, which implies the participation of the charge transfer character in the emitting state

TABLE 2
Emission lifetimes and quantum yields of 4 and 5 at room temperature in
different solvents.

λₑₘ/nm (τ/ns; Φₑₘ)

| Solvent | 4 | 5 |
|---|---|---|
| CH₂Cl₂ | 584 (—; 0.036) | 584 (—; 0.0012) |
| Acetonitrile | 576 (—; 0.022) | 574 (—; 0.0019) |
| Acetone | 579 (30; 0.0048) | — |
| Toluene | 594 (1210; 0.18) | 597 (600; 0.053) |
| DMSO | 587 (—; 0.0009) | 605 (—; 0.0021) |

The emission quantum yields of 4 and 5 are measured to be 0.18 and 0.053 in toluene, respectively, which are much higher than platinum(II) terpyridyl complexes with PTZ acetylide ligands (Φₑₘ less than 0.00033). See Chakraborty, S.; Wadas, J. T.; Hester, H.; Schmehl, R.; Eisenberg, R. Inorg. Chem. 2005, 44, 6865 (incorporated by reference herein). The emission quantum yield of 4 is approximately 4 times and 2.4 times as large as those of its corresponding platinum chloride precursor 3 and its acetylide analogues F-3 and F-4, respectively, and is 1.6 times larger than that of the “alkoxyl free” ĈN̂N platinum phenylacetylide complexes without the fluorenyl substituent (Φₑₘ=0.07). The stronger emission of 4 and 5 than that of their corresponding platinum terpyridyl PTZ acetylide dyads and triads is attributed to the nature of the emitting states for 4 and 5 as discussed in the previous two paragraphs, which admixes ³MLCT/³ILCT/³π,π* characters and thus is much less affected by the acetylide ligand. In addition, stronger emission is commonly seen from Pt(ĈN̂N) complexes than from platinum terpyridyl complexes.

Emission measured in butyronitrile glassy solution at 77 K (FIG. 5) reveals that the emission band becomes structured and blue shifted in comparison to that measured at room temperature. The vibronic spacing is 1150 cm⁻¹ for 4 and 1310 cm⁻¹ for 5, which falls in the frequency range for the in-plane bending mode of the Aryl-H bond, and the C—N stretching mode and the ring breathing mode of the pyridine rings. Considering the similar emission energy and structure of the emission of these two complexes to those reported in the literature for other platinum terpyridyl or ĈN̂N complexes, the emitting state at 77 K for 4 and 5 is tentatively assigned as the ³MLCT state, presumably mixed with some ¹ILCT and ³ π,π* character. With an increased concentration from 10⁻⁶ mol/L to ˜7×10⁻⁵ mol/L, the emission spectra remain the same, suggesting that no ground-state aggregation occurs in this concentration range. The lifetimes measured at the two vibronic bands are essentially the same, all around 10 μs (Table 1).

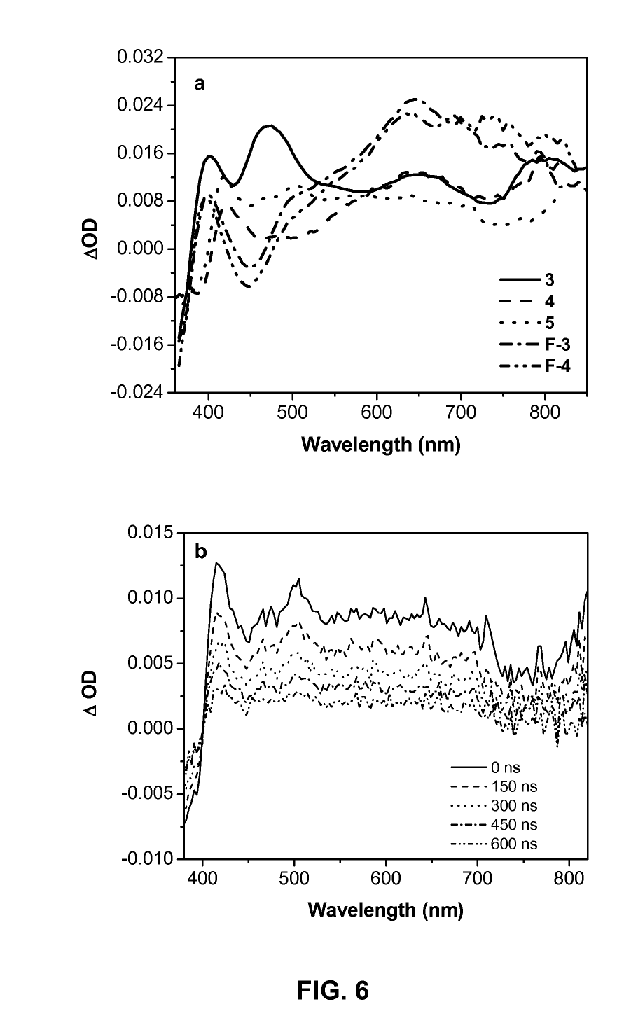

The triplet transient difference absorption spectra of 4 and 5 in toluene at zero time delay after excitation and the time-resolved triplet transient difference absorption spectra of 5 are illustrated in FIG. 6. For comparison purpose, the triplet transient difference absorption spectrum of 3 in CH₃CN is also included in FIG. 6a. The lifetimes of the triplet exited state deduced from the decay of the transient absorption are listed in Table 1. Both 4 and 5 exhibit positive absorption bands from 400 to 820 nm, indicating stronger triplet excited-state absorption than that of the ground state in this spectral region, and a bleaching band in the UV region. The relatively narrower band at ca. 420 nm for both complexes could possibly arise from the absorption of the NAN anion resulting from ³MLCT, ³ILCT, or ³LLCT. However, the lack of obvious absorption band in the region of 480-520 nm suggests that no PTZ⁺ is generated during the excitation. Therefore, contribution from the ³LLCT state could be excluded.

The broad absorption band at ca. 640 nm for complex 4 locates at the similar region to those observed in the precursor complex 3 and the acetylide analogues F-3 and F-4, suggesting that this band emanates from the ³MLCT/³ILCT states that are independent on the monodentate ligand. The lifetimes deduced from the decay of both bands are essentially the same (Table 1), indicating that the absorption originates from the same transient species. Additionally, these lifetimes are comparable to those obtained from the decay of the emission. Therefore, the transient absorption likely arises from the same excited state that emits or a state that is in equilibrium with the emitting state. Therefore, the state that gives rise to the transient absorption is tentatively assigned as the ³MLCT/³ILCT state. At the same excitation condition (Å=0.4 at 355 nm), the ΔOD value at 532 nm for 5 is approximately 2 times as large as that for 4. Therefore it is expected that the reverse saturable absorption for 5 at 532 nm would be stronger than that for 4, which is demonstrated by the nonlinear transmission measurement.

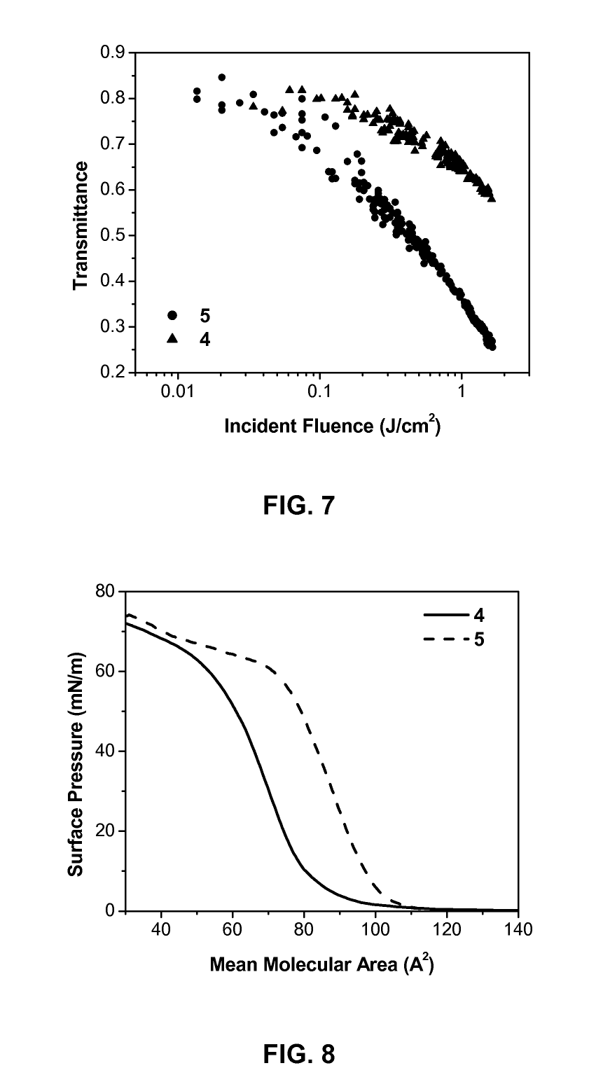

Complexes 4 and 5 exhibit broad positive triplet transient absorption in the visible to the near-IR region, and both complexes possess long-lived triplet excited state. Therefore, reverse saturable absorption (RSA) is expected to occur in the visible to the near-IR region. To demonstrate this, nonlinear transmission measurements were performed at 532 nm using nanosecond laser pulses. FIG. 7 shows the results of 4 and 5 in toluene solutions at a linear transmission of 80% in a 2-mm cuvette. Both of the complexes show significant transmission decrease with increased incident fluence, which is a typical phenomenon for RSA. However, the RSA of 5 is much stronger than that of 4. When the incident fluence is increased to 1.6 J/cm², the transmission decreases to 58% for 4 and 25% for 5. The stronger RSA of 5 could be ascribed to its smaller ground-state absorption cross-section (σ₀=1.4×10⁻¹⁸ cm²) compared to that of 4 (σ₀=2.1×10⁻¹⁸ cm²) at 532 nm. Meanwhile, the triplet excited-state absorption cross-sections at 532 nm are estimated according to the equation ΔOD=(ε_{T}−ε_{g})C_{T}l, where ε_{T} and ε_{g} are the triplet excited state and ground state extinction coefficients, respectively, C_{T} is the triplet excited state concentration, and l is the optical pathlength of the sample. Since the ε_{T} at the TA band maximum has been measured by the partial saturation method, the ε_{g} can be deduced from the UV-vis measurement, ΔOD can be obtained from the transient difference absorption spectrum, and the pathlength for the sample is 1 cm, the triplet excited state concentration c_{T} can thus be estimated. Using this value and the ΔOD and ε_{g} obtained from the TA spectrum and the UV-vis absorption spectrum at 532 nm, respectively, the ε_{T}'s at 532 nm are estimated to be 1020 L·mol⁻¹·cm⁻¹ for 4 and 2720 L·mol⁻¹·cm⁻¹ for 5, which correspond to an excited-state absorption cross-section (σ_{T}) of 3.9×10⁻¹⁸ cm² for 4 and 1.0×10⁻¹⁷ cm² for 5 according to the conversion equation σ=2303ε/N_{A}, where N_{A} is the Avogadro constant. Shao, P.; Li, Y.; Sun, W. Organometallics 2008, 27, 2743 (incorporated by reference herein). Although this is a rough estimation of the excited-state absorption cross-section using this method, the resultant ratio of σ_{T}/σ₀, i.e. 1.9 for 4 and 7.4 for 5 at 532 nm, still corresponds well with the observed RSA trend for these two complexes.

Platinum(II) ĈN̂N complexes with hydrophilic substituents can form LB films due to their amphiphilic property. To explore whether complexes 4 and 5 can form LB films, surface pressure-mean molecular area of these two complexes have been measured and the results are presented in FIG. 8. The isotherm indicates that the monolayers start to lift around the same molecular area of ca. 110 Å² for both 4 and 5, after which the surface pressure increases sharply. The slopes of the isotherm corresponding to the liquid condensed phase are essentially the same for 4 and 5, indicating the similar liquid condensed state for these two complexes. Both complexes collapse at a surface pressure of ca. 70 mN/m, but the collapsing pressure for 5 is slightly higher than that for 4, indicating that 5 forms a slightly more stable monolayer than 4 does. The limiting molecular areas obtained by extrapolating the plot to zero surface pressure are approximately 86 and 103 Å² for monolayers of 4 and 5, respectively, which are much smaller than the estimated molecular areas (448 Å² for 4 and 384 Å² for 5, obtained by geometry optimization through density functional theory (DFT) calculation in vacuum using the DMOL³ program implemented in Material Studio 4.3). This suggests that the molecules take up an “edge-on” orientation rather than a “flat-on” arrangement. With reference to LB films of the mononuclear ĈN̂N complex with alkoxyl substituent, the alkyl chain in complexes 4 and 5 should stick out from the air/water interface.

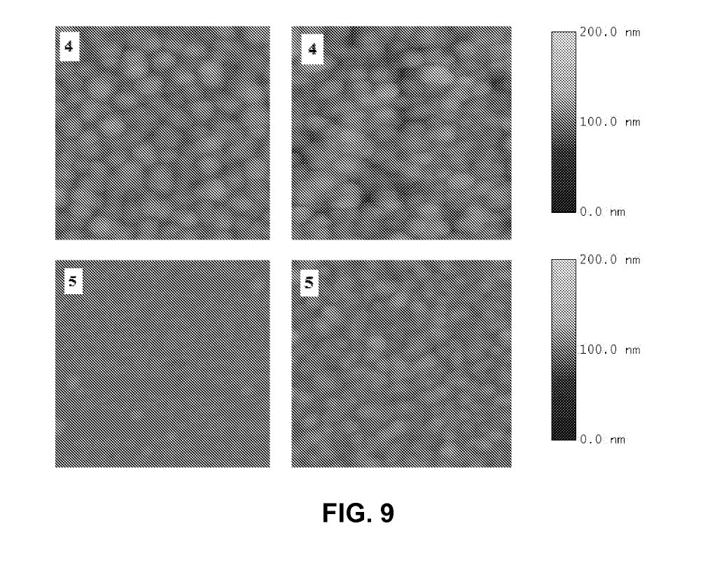

FIG. 9 shows the AFM height images of 5-layer and 11-layer LB films of 4 and 5, which illustrate the surface morphology of the films. Both the 5-layer and 11-layer LB films of 5 are smoother than those of 4. Particularly the 5-layer film of 5 is more uniform and fewer grains are observed in its AFM image. This indicates that the 5-layer film is more stable and organized than the 11-layer film because defects build up when the number of layer increases. The bright grain-like particles could be ascribed to the formation of aggregation in LB films, which is more salient in the LB films of 4 in comparison to that in 5. The ease of aggregation in LB films of 4 than in 5 could be related to the presence of phenyl ring in the acetylide ligand of 4, which could facilitate π,π stacking in the solid form.

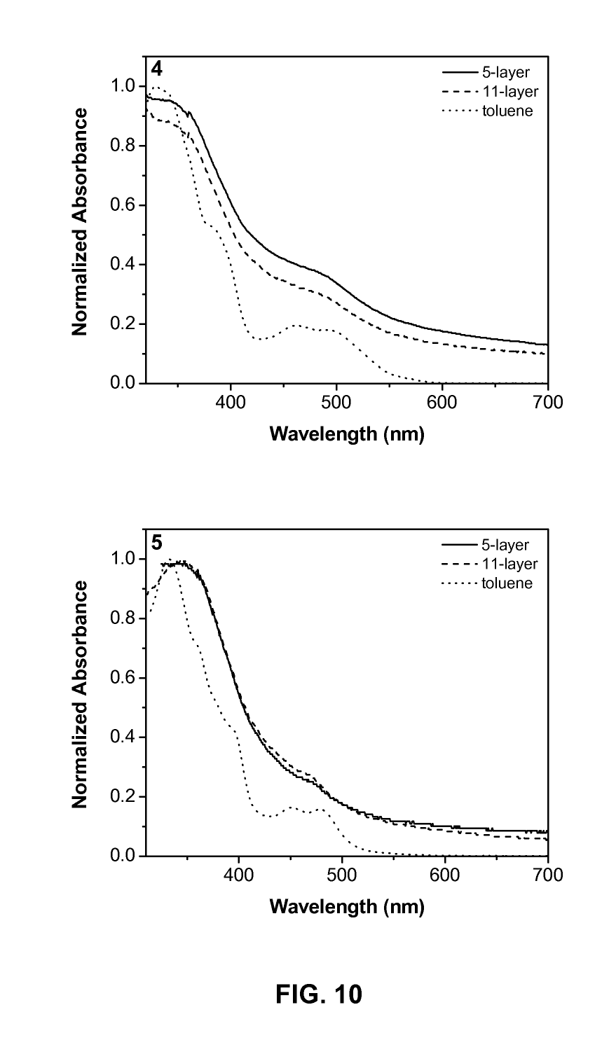

The UV-vis absorption spectra of the LB films of 4 and 5 are shown in FIG. 10. For comparison purpose, their UV-vis absorption spectra in toluene are also provided in the same figure. The ¹π,π* transitions in the UV region became a broad and structureless absorption band in the films, and the charge-transfer band became a shoulder at ca. 480 nm.

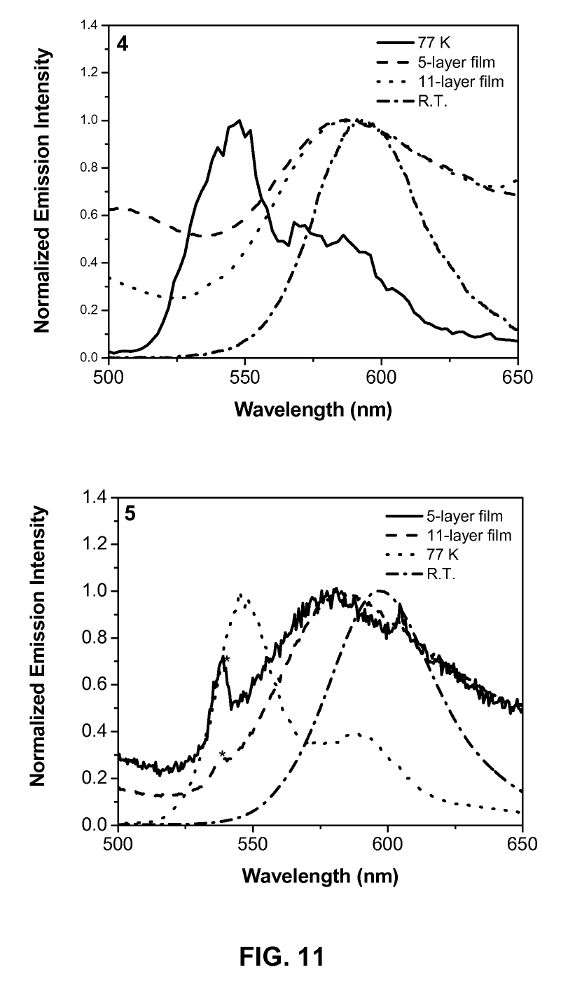

The LB films of both complexes are emissive at room temperature. The emission spectra for the 5-layer and 11-layer LB films are shown in FIG. 11, along with the spectra in toluene at room temperature and the one in butyronitrile glassy solution at 77 K. The number of layers shows a negligible effect on the emission energy of the LB films, and the energy is comparable to that in toluene solution, with a slight blue-shift. Although aggregation was observed via the AFM image in the LB films, the intermolecular distance is probably not close enough in the aggregates to allow for Pt—Pt interactions to occur. Therefore, no red-shifted emission attributing to the metal-metal-to-ligand charge transfer (³MMLCT) is observed, which is in line with that observed in the UV-vis absorption spectra.

Introducing PTZ acetylide ligands to platinum (II) complexes causes a mixture of ¹LLCT character into the ¹MLCT/¹ILCT state due to the electron-donating ability of the PTZ acetylide ligand, which results in a broadening and slight red-shift of the lowest-energy charge-transfer band of 4 and 5 compared to that of their corresponding platinum chloride precursor 3. Both complexes are emissive at room temperature in fluid solutions and at 77 K in glassy matrix; with the emission quantum yield of 4 (Φₑₘ=0.18) at room temperature in toluene is larger than that of 5 (Φₑₘ=0.053). Both complexes exhibit broadband triplet excited-state absorption, therefore considerable reverse saturable absorption (RSA) was observed at 532 nm for ns laser pulses. Due to the smaller ground-state absorption cross-section but larger triplet excited-state absorption at 532 nm for 5 compared to those of 4, 5 exhibits stronger RSA than 4 does. The presence of the alkyl chain on the ĈN̂N ligand makes it possible to fabricate LB films for 4 and 5. Although aggregation is evident in the LB films of these two complexes via AFM images, the electronic absorption energy and the emission energy are comparable to those in solutions, indicating that the intermolecular distance in the LB films is not close enough for intermolecular Pt—Pt interaction to occur.

### Example 3

### Cyclometalated Platinum(II) 6-Phenyl-4-(9,9-dihexylfluoren-2-yl)-2,2′-bipyridine Complexes: Synthesis, Photophysics and Nonlinear Absorption

### Platinum Complexes

In this example, two new ĈN̂N ligands containing the fluorene unit at the 4-position of the central pyridine ring and their platinum complexes with different monodentate co-ligands were synthesized and characterized. Structures of these complexes and their synthetic routes are displayed in Scheme 2. The photophysics of these complexes and their reverse saturable absorption for ns laser pulses at 532 nm were systematically investigated and analyzed.

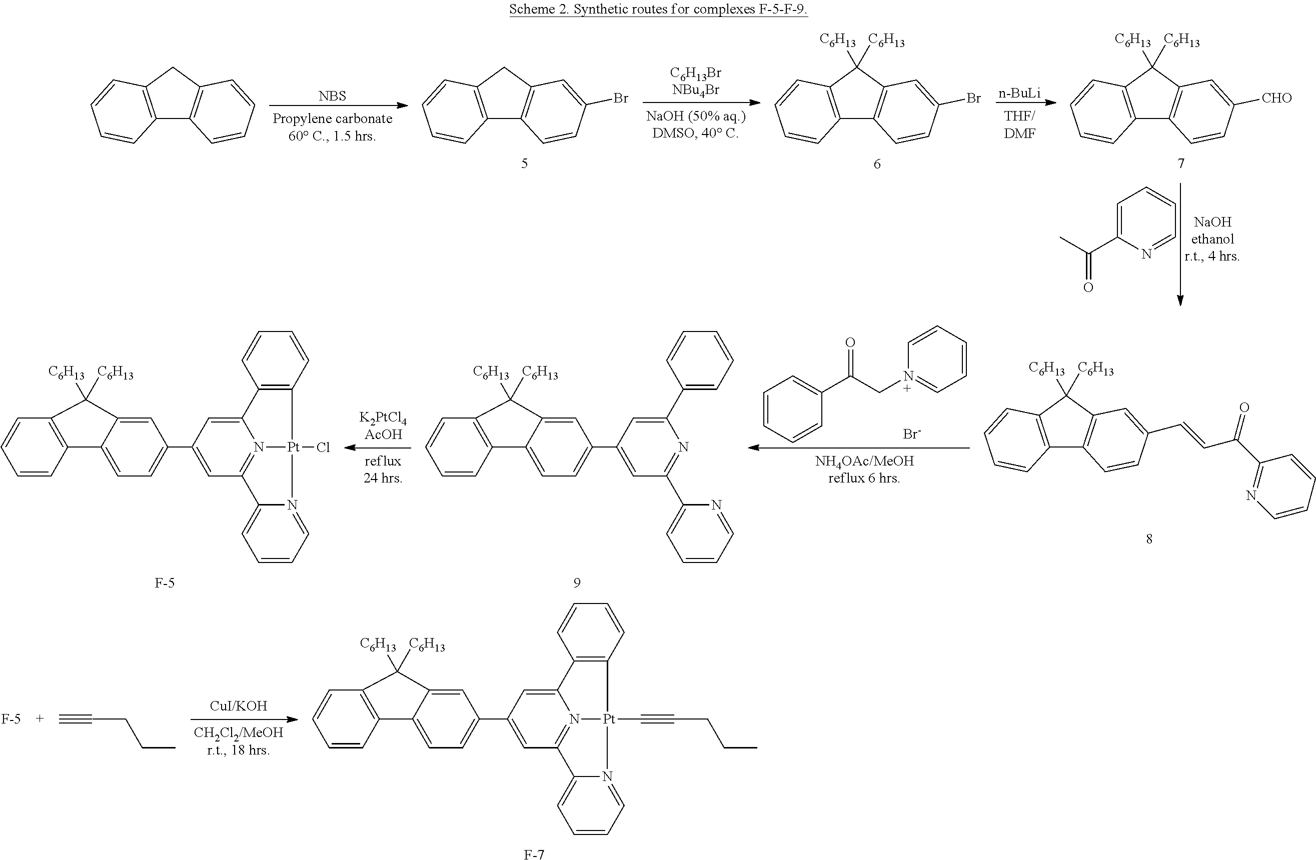

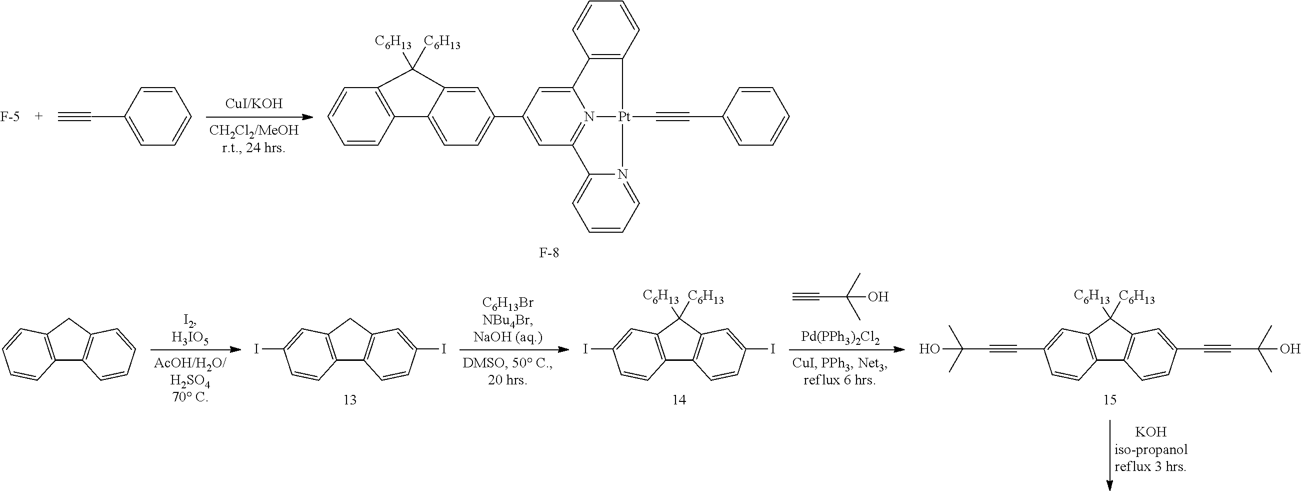

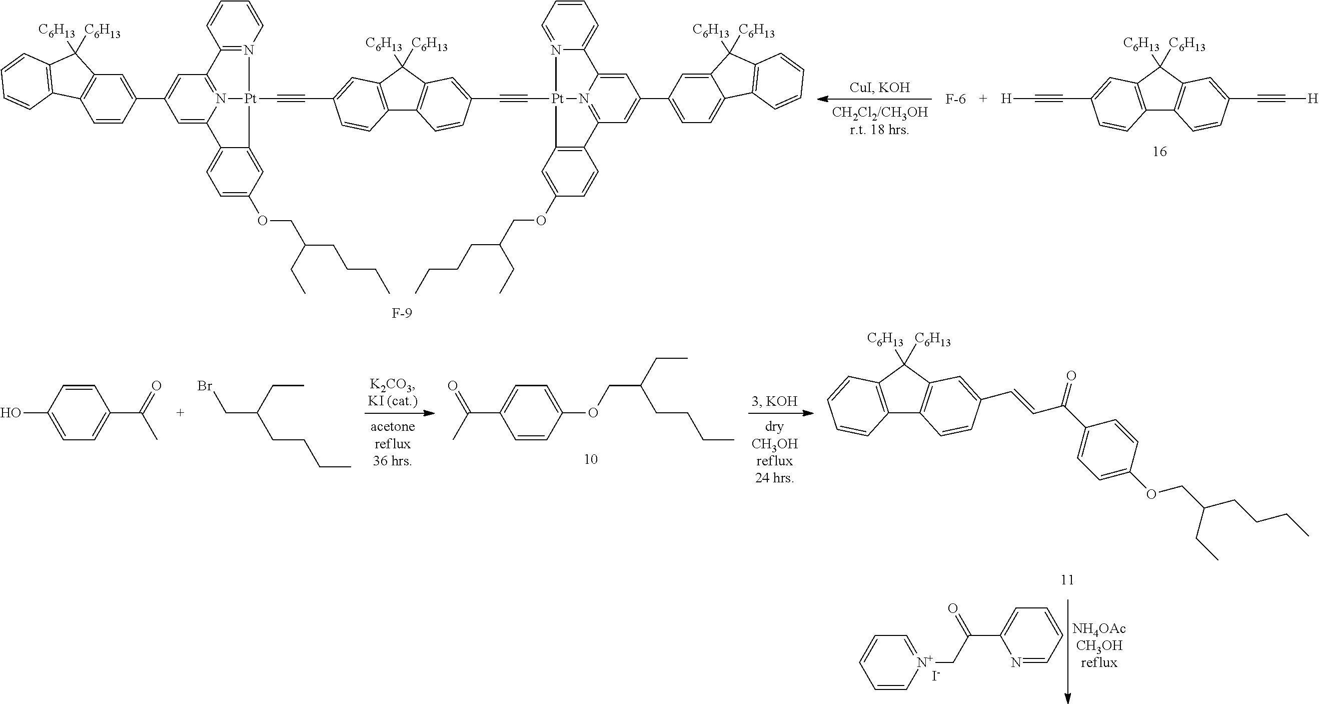

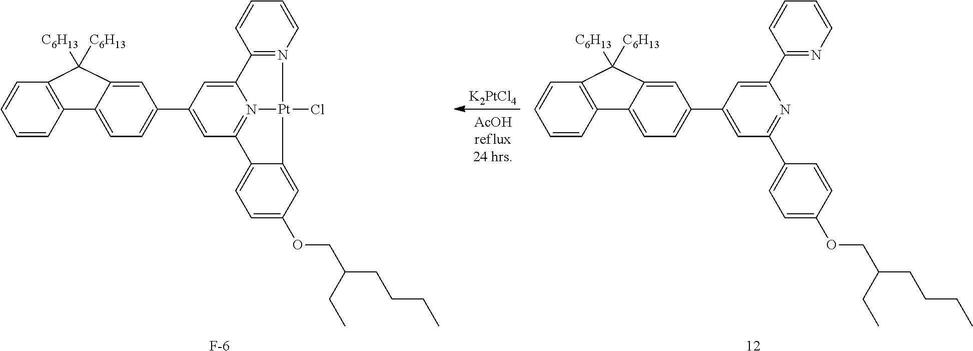

The compounds pyridacylpyridinium iodide (Neve, F.; Crispini, A.; Campagna, S.; Serroni, S. Inorg. Chem. 1999, 38, 2250 (incorporated by reference herein)), 5-6 (Koizumi, Y.; Seki, S.; Tsukuda, S.; Sakamoto, S.; Tagawa, S. J. Am. Chem. Soc. 2006, 128, 9036 (incorporated by reference herein)), 7 (He, F.; Xia, H.; Tang, S.; Duan, Y.; Zeng, M.; Liu, L.; Li, M.; Zhang, H.; Yang, B.; Ma, Y.; Liu, S.; Shen J. J. Mater. Chem. 2004, 14, 2735 (incorporated by reference herein)), 10 (Shao, P.; Li, Y.; Azenkeng, A.; Hoffmann, M.; Sun, W., supra) and 13-16 (Lee, S. H.; Nakamura, T.; Tsutsui, T. Org. Lett. 2001, 3, 2005 (incorporated by reference herein)) were prepared according to the literature procedures. All the solvents and reagents were purchased from Alfa Aesar and used as received unless otherwise stated. Column chromatography was carried out on silica gel (Sorbent Technologies, 60 Å, 230×450 mesh) or neutral aluminum oxide (Sigma-Aldrich, 58 Å, ˜150 mesh).

¹H NMR spectra were measured on a Varian 300 or 400 MHz VNMR spectrometer. High-resolution mass spectrometry was carried out on a Bruker BioTOF III mass spectrometer. Elemental analyses were carried out by NuMega Resonance Laboratories, Inc. in San Diego, Calif.

8. To a mixture of 7 (1.43 g, 4 mmol) and 2-acetylpyridine (0.48 g, 4 mmol) in 20 mL ethanol, 4 mL of 1.5 M aqueous NaOH solution was added. After stirring at room temperature for 4 hrs, the solid formed was collected by filtration. The crude product was purified by recrystallization from methanol to afford 0.21 g yellow solid as the pure product (yield: 11%). ¹H NMR (CDCl₃): δ 8.79 (d, J=4.5 Hz, 1H), 8.32 (d, J=16.2 Hz, 1H), 8.23 (d, J=8.1 Hz, 1H), 8.06 (d, J=16.2 Hz, 1H), 7.88-7.93 (m, 1H), 7.72 (d, J=5.1 Hz, 4H), 7.50-7.54 (m, 1H), 7.35 (d, J=3.3 Hz, 3H), 1.98-2.04 (m, 4H), 1.04-1.14 (m, 12H), 0.75 (t, J=6.6 Hz, 6H), 0.59-0.63 (m, 4H) ppm. ESI-HRMS: m/z calcd for [C₃₃H₃₉NO+H]⁺: 466.3104; found, 466.3124. Anal. Calcd (%) for C₃₃H₃₉NO: C, 85.11; H, 8.44; N, 3.01. Found: C, 84.75; H, 8.90; N, 3.05.

9. A mixture of 8 (1.42 g, 3 mmol), N-(benzoylmethyl)pyridinium bromide (0.85 g, 3 mmol) and NH₄OAc (3.00 g, 39 mmol) in 70 mL methanol was refluxed for 6 hrs. Methanol was removed and 40 mL water was added to the residue. The resultant mixture was extracted with CH₂Cl₂. The organic layer was washed with brine (40 mL×2) and dried over Na₂SO₄. The crude product was purified using a silica gel column eluted with hexane/ethyl acetate (v/v=20/1) to yield 0.75 g colorless viscous oil (yield: 44%). ¹H NMR (CDCl₃): δ 8.77-8.80 (m, 1H), 8.73-8.76 (m, 2H), 8.29 (dd, J=1.5, 8.4 Hz, 2H), 8.09 (d, J=1.5 Hz, 1H), 7.91 (dd, J=2.1, 7.5 Hz, 1H), 7.86-7.89 (m, 2H), 7.83 (d, J=1.2 Hz, 1H), 7.78-7.81 (m, 1H), 7.59 (td, J=1.5, 6.6 Hz, 2H), 7.47-7.53 (m, 1H), 7.35-7.44 (m, 4H), 2.02-2.13 (m, 4H), 1.11-1.19 (m, 12H), 0.73-0.82 (m, 10H) ppm. ESI-HRMS: m/z calcd for [C₄₁H₄₄N₂+H]⁺: 565.3577; found, 565.3572. Anal. Calcd (%) for C₄₁H₄₄N₂: C, 87.19; H, 7.85; N, 4.96. Found: C, 86.91; H, 8.05; N, 4.97.

11. A mixture of 7 (1.17 g, 3.23 mmol), 10 (0.80 g, 3.23 mmol) and KOH (0.84 g, 15 mmol) in dry MeOH (80 mL) was refluxed for 24 hrs. The solvent was removed and 40 mL water was added to the residue. The resultant mixture was extracted with ether. The organic layer was washed with brine and dried over Na₂SO₄. The crude product was purified by a silica gel column eluted with CH₂Cl₂/hexane (v/v=1/1) to afford 0.71 g yellow oil (yield: 37%). ¹H NMR (CDCl₃): δ 8.09 (d, J=8.4 Hz, 2H), 7.93 (d, J=15.6 Hz, 1H), 7.71-7.73 (m, 2H), 7.59-7.66 (m, 3H), 7.34 (d, J=2.4 Hz, 3H), 7.00 (d, J=8.4 Hz, 2H), 3.93 (d, J=5.4 Hz, 2H), 1.98-2.04 (m, 4H), 1.75-1.77 (m, 1H), 1.33-1.54 (m, 8H), 1.05-1.14 (m, 12H), 0.86-0.97 (m, 6H), 0.73-0.78 (m, 6H), 0.63 (s, 4H) ppm. ESI-HRMS: m/z calcd for [C₄₂H₅₆O₂+H]⁺: 593.4353; found, 593.4363. Anal. Calcd (%) for C₄₂H₅₆O₂.C₆H₁₄: C, 84.89; H, 10.40. Found: C, 85.35; H, 10.70.

12. A mixture of 11 (1.46 g, 2.5 mmol), pyridacylpyridinium iodide (0.80 g, 2.5 mmol) and NH₄OAc (2.00 g, 26 mmol) was refluxed in 50 mL MeOH for overnight. The solvent was removed and 40 mL ether was added to the residue. The resultant mixture was washed with brine and dried over Na₂SO₄. The crude product was purified by a silica gel column eluted with CH₂Cl₂/hexane (v/v=1/1) to give 0.22 g colorless viscous oil (yield: 13%). ¹H NMR (CDCl₃): δ 8.73 (d, J=4.8 Hz, 1H), 8.68 (d, J=7.8 Hz, 1H), 8.61 (d, J=1.2 Hz, 1H), 8.17 (d, J=8.7 Hz, 2H), 7.97 (d, J=1.2 Hz, 1H), 7.84-7.90 (m, 1H), 7.80 (t, J=1.2 Hz, 2H), 7.73-7.76 (m, 2H), 7.32-7.37 (m, 4H), 7.05 (d, J=9.0 Hz, 2H), 3.93 (d, J=5.7 Hz, 2H), 2.00-2.05 (m, 4H), 1.73-1.80 (m, 1H), 1.33-1.55 (m, 8H), 1.05-1.14 (m, 12H), 0.91-0.97 (m, 6H), 0.75 (t, J=6.3 Hz, 6H), 0.64-0.66 (m, 4H) ppm. ESI-HRMS: m/z calcd for [C₄₉H₆₀N₂O+H]⁺: 693.4778; found, 693.4809. Anal. Calcd (%) for C₄₉H₆₀ON₂.⅓CH₂Cl₂: C, 82.14; H, 8.48; N, 3.88. Found: C, 82.23; H, 8.27; N, 3.56.

F-5. A mixture of ligand 9 (0.20 g, 0.35 mmol) and K₂PtCl₄ (0.15 g, 0.35 mmol) was refluxed in 60 mL AcOH for 24 hrs. After cooled to room temperature, the yellow precipitant was collected by filtration. The crude product was purified by a neutral alumina gel column eluted with CH₂Cl₂, and then further purified by recrystallization from CH₂Cl₂/ether. Orange solid was obtained as the pure product (0.23 g, yield: 83%). ¹H NMR (CDCl₃): δ 8.73 (d, J=5.4 Hz, 1H), 7.92 (d, J=3.6 Hz, 2H), 7.85 (d, J=8.4 Hz, 1H), 7.78-7.81 (m, 1H), 7.72-7.74 (m, 2H), 7.60 (d, J=1.8 Hz, 1H), 7.44 (dd, J=7.2, 1.2 Hz, 1H), 7.37-7.41 (m, 3H), 7.31 (t, J=4.2 Hz, 2H), 7.22-7.24 (m, 1H), 6.96 (td, J=1.2, 7.8 Hz, 1H), 6.88 (td, J=1.2, 7.5 Hz, 1H), 2.07-2.16 (m, 4H), 1.09-1.14 (m, 12H), 0.76 (t, J=6.6 Hz, 6H), 0.69 (br. s, 4H) ppm. ESI-MS: m/z calcd for [C₄₁H₄₃N₂¹⁹⁵Pt+CH₃CN]⁺, 799.3338; found, 799.3396. Anal. Calcd (%) for C₄₁H₄₃ClN₂Pt: C, 61.99; H, 5.46; N, 3.53. Found: C, 62.30; H, 5.10; N, 3.64.

F-6. A mixture of 12 (0.25 g, 0.36 mmol) and K₂PtCl₄ (0.15 g, 0.36 mmol) was refluxed in 60 mL AcOH for 24 hrs. After cooled down to room temperature, the solid formed was collected by filtration. The crude product was purified by a neutral alumina gel column eluted by CH₂Cl₂, and then recrystallized from CH₂Cl₂/hexane. 0.25 g orange solid was obtained as the pure product (yield: 76%). ¹H NMR (CDCl₃): δ 8.72 (d, J=5.1 Hz, 1H), 7.77-7.82 (m, 5H), 7.71 (d, J=8.1 Hz, 1H), 7.46 (s, 1H), 7.36-7.39 (m, 3H), 7.18-7.23 (m, 2H), 7.15 (d, J=8.4 Hz, 1H), 6.98 (d, J=2.4 Hz, 1H), 6.49 (d, J=8.4 Hz, 1H), 3.72 (d, J=5.7 Hz, 2H), 2.06-2.12 (m, 4H), 1.67-1.72 (m, 1H), 1.26-1.51 (m, 8H), 1.05-1.15 (m, 12H), 0.85-0.90 (m, 6H), 0.72-0.79 (m, 10H) ppm. ESI-MS: m/z calcd for [C₄₉H₅₉N₂O¹⁹⁵Pt+CH₃CN]⁺, 927.4539; found, 927.4551. Anal. Calcd (%) for C₄₉H₅₉ClN₂OPt: C, 63.79; H, 6.45; N, 3.04. Found: C, 63.82; H, 6.22; N, 3.35.

F-7. A mixture of F-5 (0.13 g, 0.16 mmol), 1-pentyne (40 μL, 0.41 mmol), CuI (3.0 mg, 0.01 mmol) and KOH (60 mg, 1 mmol) in degassed CH₂Cl₂/CH₃OH (40 mL/20 mL) was stirred at room temperature under argon for 18 hrs. After the reaction, the solvent was removed and the crude product was purified by a neutral alumina gel column eluted by CH₂Cl₂/hexane (v/v=3/1), and then recrystallized from CH₃OH. Red solid was obtained as the pure product (89 mg, yield: 67%). ¹H NMR (CDCl₃): δ 9.05 (d, J=5.4 Hz, 1H), 7.92-7.98 (m, 2H), 7.69-7.86 (m, 5H), 7.60 (s, 1H), 7.53 (s, 1H), 7.34-7.38 (m, 5H), 6.96-7.05 (m, 2H), 2.68 (t, J=7.2 Hz, 2H), 2.02-2.08 (m, 4H), 1.67-1.77 (m, 2H), 1.07-1.17 (m, 15H), 0.67-0.77 (m, 10H) ppm. ESI-MS: m/z calcd for [C₄₆H₅₀N₂¹⁹⁵Pt+H]⁺, 826.3698; found, 826.3695. Anal. Calcd (%) for C₄₆H₅₀N₂Pt: C, 66.89; H, 6.10; N, 3.39. Found: C, 66.51; H, 6.37; N, 3.54.

F-8. A mixture of F-5 (0.13 g, 0.16 mmol), phenylacetylene (24 μL, 0.24 mmol), CuI (3.0 mg, 0.01 mmol) and KOH (60 mg, 1.00 mmol) in degassed CH₂Cl₂/CH₃OH (50 mL/25 mL) was stirred at room temperature under argon for 24 hrs. The solvent was removed and the crude product was purified by a neutral alumina gel column with CH₂Cl₂/hexane (v/v=3/1) used as the eluent. Recrystallization from CH₂Cl₂/ether yields 82 mg red solid as the pure product (yield: 59%). ¹H NMR (CDCl₃): δ 9.10 (d, J=4.8 Hz, 1H), 8.01 (d, J=4.4 Hz, 2H), 7.75-7.81 (m, 4H), 7.71 (d, J=8.0 Hz, 1H), 7.64 (s, 1H), 7.58 (d, J=8.0 Hz, 3H), 7.37-7.43 (m, 6H), 7.29 (t, J=8.0 Hz, 2H), 7.18-7.20 (m, 1H), 7.03 (dd, J=3.2, 5.6 Hz, 1H), 2.01-2.05 (m, 4H), 1.07-1.14 (m, 12H), 0.76 (t, J=7.2 Hz, 6H), 0.66 (br. s, 4H) ppm. ESI-MS: m/z calcd for [C₄₉H₄₈N₂¹⁹⁵Pt+Na]⁺, 882.3362; found, 882.3343. Anal. Calcd (%) for C₄₉H₄₈N₂Pt.0.1CH₂Cl₂: C, 67.90; H, 5.59; N, 3.23. Found: C, 67.70; H, 5.26; N, 3.38.

F-9. A mixture of F-6 (0.48 g, 0.52 mmol), 16 (0.10 g, 0.26 mmol), CuI (6.0 mg, 0.02 mmol) and KOH (56 mg, 1.0 mmol) in degassed CH₂Cl₂/CH₃OH (50 mL/40 mL) was stirred at room temperature under argon for 18 hrs. The solvent was removed and the crude product was purified by a neutral alumina gel column (CH₂Cl₂/hexane (v/v=3/1) was used as the eluent), and then recrystallized from CH₂Cl₂/ether. Red solid was obtained as the pure product (0.34 g, yield: 30%). ¹H NMR (CDCl₃): δ 9.38 (d, J=5.7 Hz, 2H), 8.05 (s, 4H), 7.57-7.82 (m, 22H), 7.45 (d, J=8.4 Hz, 2H), 7.39 (br. s, 6H), 6.65 (d, J=9.0 Hz, 2H), 4.03 (br. s, 4H), 2.02 (br. s, 12H), 1.78 (br. s, 2H), 1.35-1.52 (m, 16H), 1.08-1.23 (m, 36H), 0.91-0.98 (m, 12H), 0.75-0.78 (m, 30H) ppm. ESI-MS: m/z calcd for [C₁₂₇H₁₅₀N₄O₂Pt₂+2Na]²⁺, 1100.0432; found, 1100.0466. Anal. Calcd (%) for C₁₂₇H₁₅₀N₄O₂Pt₂: C, 70.79; H, 7.02; N, 2.60. Found: C, 70.73; H, 6.69; N, 2.64.

### Photophysical Measurements

The UV-vis absorption spectra were measured using an Agilent 8453 spectrophotometer in a 1-cm or 1-mm quartz cuvette. The steady-state emission spectra were obtained on a SPEX fluorolog-3 fluorometer/phosphorometer. The emission quantum yields were determined by the optical dilute method (Demas, J. N.; Crosby, G. A. J. Phys. Chem. 1971, 75, 991 (incorporated by reference herein)) in degassed solutions, and a degassed aqueous solution of [Ru(bpy)₃]Cl₂ (Φₑₘ=0.042, λₑₓ=436 nm) (Van Houten, J.; Watts, R. J. Am. Chem. Soc. 1976, 98, 4853 (incorporated by reference herein)) was used as the reference. The excited-state lifetimes, the triplet excited-state quantum yields, and the triplet transient difference absorption spectra were measured in degassed solutions on an Edinburgh LP920 laser flash photolysis spectrometer. The third harmonic output (355 nm) of a Nd:YAG laser (Quantel Brilliant, pulsewidth ˜4.1 ns, repetition rate was set at 1 Hz) was used as the excitation source. Each sample was purged with Ar for 30 minutes before measurement.

The self-quenching rate constants (k_{Q}) in CH₂Cl₂ were deduced following the Stern-Volmer equation:

k_{obs}=k_{Q}[C]+k₀ (1)

where k_{obs} is the decay rate constant of the emission (k_{obs}=1/τₑₘ), k_{Q} is the self-quenching rate constant, [C] is the concentration of the complex in mol/L, and k₀ (k₀=1/τ₀) is the decay rate constant of the excited-state at infinite dilute solution. A plot of the observed decay rate constant versus concentration should give rise to a straight line. The slope of the straight line corresponds to k_{Q} and the intercept corresponds to k₀.

The triplet excited-state molar extinction coefficient and triplet quantum yield were determined by the partial saturation method. Carmichael, I.; Hug, G. L. J. Phys. Chem. Ref Data 1986, 15,1 (incorporated by reference herein). The optical density at 585 nm was monitored when the excitation energy at 355 nm was gradually increased. Saturation was observed when the excitation energy was higher than 10 mJ. The following equation was then used to fit the experimental data to obtain the ε_{T} and Φ_{T}. Id.

ΔOD=a(1−exp(−bIₚ)) (2)

where ΔOD is the optical density at 585 nm, Iₚ is the pump intensity in Einstein·cm⁻², a=(ε_{T}−ε₀)dl, and b=2303 ε₀^{ex} _{T}/Å. ε_{T} and ε₀ are the absorption coefficients of the excited state and the ground state at 585 nm, ε₀^{ex} is the ground state absorption coefficient at the excitation wavelength of 355 nm, d is the concentration of the sample (mol·L⁻¹), l is the thickness of the sample, and A is the area of the sample irradiated by the excitation beam.

### Nonlinear Transmission Measurement

The nonlinear transmission measurement experimental setup was similar to that described previously (F. Guo, W. Sun, Y. Liu, K. Schanze, Inorg. Chem. 2005, 44, 4055 (incorporated by reference herein)), with a 20-cm focal length lens used to focus the beam to the 2-mm thick sample cuvette. A Q-switched Quantel Brilliant Nd:YAG laser operated at the second-harmonic wavelength (532 nm) was used as the light source. The repetition rate of the laser was 10 Hz. Energies of the incident laser beam were attenuated by a combination of a half-wave plate and a polarizer. The beam was then split by a wedged beamsplitter. One of the reflected beams was used to monitor the incident energy. The diameter of the transmitted beam was reduced to half of the original size by a telescope and was focused by a 20-cm plano-convex lens (f/52.5) to the center of a 2-mm sample cell. The radius of the beam waist was approximately 17.8 μm. The incident energy and the output energy were monitored by two Molectron J4-09 pyroelectric joule meters.

### Z-Scan Measurements

The open-aperture Z-scan measurements experimental setup is similar to the one reported previously. Li, Y.; Pritchett, T. M.; Huang, J.; Ke, M.; Shao, P.; Sun, W. J. Phys. Chem. A 2008, 112, 7200 (incorporated by reference herein). For ns Z-scan measurements, the second harmonic output (532 nm) from a Quantel Brilliant Nd:YAG laser with a pulsewidth of 4.1 ns and a repetition rate of 10 Hz was used as the light source. The laser beam was focused by a 30-cm focal-length plano-convex lens to a beam waist of 30 μm at the focal point, which corresponds to a Rayleigh length (

$${z}_{0} = {πω}_{0}^{2} / λ ,$$

where ω₀ is the radius at the beam waist) of 5.31 mm. For ps Z scans, the light source was the second harmonic output of an EKSPLA PL2143A passively mode-locked, Q-switched Nd:YAG laser (pulsewidth=21 ps, repetition rate=10 Hz). A 15-cm plano-convex lens was used to focus the beam to a beam waist of 34 μm at the focal point, which gave rise to a Rayleigh length of 6.82 mm. Therefore, the sample solution placed in a 1-mm thick quartz cuvette for ns measurements and in a 2-mm cuvette for ps measurements could be considered as thin samples. In both nanosecond and picosecond Z-scan measurements, the laser beam was split by a wedged beamsplitter. One of the reflected beams was used to monitor the incident energy, while the transmitted beam was focused by the lens to the sample cell. The sample was mounted on a translation stage (Newport M-UTM100) and moved through the vicinity of the focal plane. A computer was used to control the translation stage movement and the data acquisition. A 50-cm plano-convex lens was placed at approximately 30 cm after the linear focal plane to collect all of the transmitted light into the Molectron J4-09 joulemeter probe.

A five-band model that consists of a ground state, two singlet excited states and two triplet excited states was used to fit the Z-scan experimental data. The detailed descriptions of the model and the fitting procedure were reported previously. Li, Y.; Pritchett, T. M.; Huang, J.; Ke, M.; Shao, P.; Sun, W. J. Phys. Chem. A 2008, 112, 7200 (incorporated by reference herein).

### Results

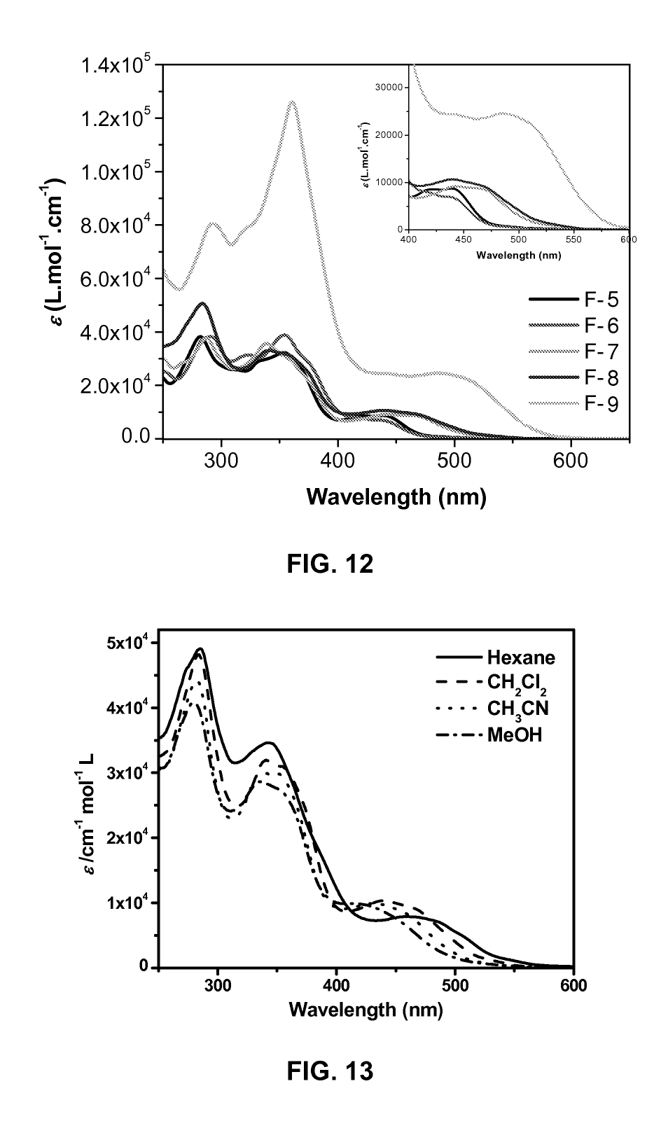

The UV-vis absorption spectra of F-5-F-9 were investigated in CH₂Cl₂ at different concentrations (5×10⁻⁶ to 5×10⁻⁴ mol/L), and the results are presented in FIG. 12 for a concentration of 5×10⁻⁵ mol/L. The absorption of all five complexes obeys Lambert-Beer's law in the concentration range studied, indicating that no ground-state aggregation occurs in this concentration range. The intense absorption below 400 nm for these complexes could be assigned to the intraligand π,π* transitions within the 4-fluorenyl-ĈN̂N ligand because these bands are essentially independent of the monodentate co-ligand. The broad, moderately intense absorption band in the visible region, i.e. 400-500 nm for F-5 and F-6, 410-550 nm for F-7 and F-8, and 420-600 nm for F-9, could be tentatively attributed to the admixture of ¹MLCT (metal-to-ligand charge transfer)/¹ILCT (intraligand charge transfer) transitions for F-5 and F-6, and ¹MLCT/¹ILCT/¹LLCT (ligand-to-ligand charge transfer) for F-7-F-9 considering the similar shape and energy of this band to those reported in the literature for other platinum ĈN̂N and terpyridyl acetylide complexes. Fan, Y.; Zhang, L.-Y.; Dai, F.-R.; Shi, L.-X.; Chen, Z.-N. Inorg. Chem. 2008, 47, 2811 (incorporated by reference herein). The involvement of the intraligand charge transfer (¹ILCT) character into the charge-transfer band of these complexes should be taken into account because of the π-donating ability of the fluorenyl unit on the ĈN̂N ligand. In addition, the charge transfer band of these complexes has enhanced intensity as compared to their respective platinum ĈN̂N complexes without the 4-fluorenyl substituent. The charge transfer band becomes broadened and red-shifted for F-7-F-9 comparing to those of F-5 and F-6. Though not wishing to be bound by a particulart theory, such a feature could be rationalized by two factors: First, the stronger π-donating ability of the acetylide in F-7-F-9 could raise the Pt d orbitals, and thus decreases the energy gap between the bipyridine-based lowest unoccupied molecular orbital (LUMO) and the d orbital. This would consequently decrease the energy of the ¹MLCT excited state. Secondly, the acetylide ligand would admix more LLCT character into the lowest excited state with the increased π-donating ability of the acetylide ligand, which would also cause the broadening and red-shift of the charge-transfer band.

The influence of the 4-fluorenyl substituent on the UV-Vis absorption spectra of F-5-F-9 is notable. The transition energies in these complexes are quite similar to those of their corresponding platinum ĈN̂N complexes without the 4-fluorenyl substituent, especially for the lowest-energy charge transfer bands, indicating that the fluorenyl substituent has a negligible effect on the energy level of the bipyridine based LUMO (π*(N̂N)) for the Pt(ĈN̂N)X complexes. However, the presence of the fluorenyl substituent significantly increases the molar extinction coefficients for the bands above 300 nm. This phenomenon likely arises from the extended π-system in the fluorenyl-substituted complexes, which could increase the oscillator strength for these transitions by increasing the transition dipoles.

The extinction coefficients for the dinuclear complex F-9 are much larger than those of the mononuclear complexes F-5-F-8. The extinction coefficients of the bands at ca. 360 nm and 490 nm are more than double of the respective bands in the mononuclear complexes, and the ε value at the low-energy absorption band apex is approximately 2.4×10⁴ M⁻¹ cm⁻¹.

The charge transfer nature of the low-energy absorption bands in F-5-F-9 is supported by the solvent-dependency studies. As exemplified in FIG. 13 for F-8, the low-energy absorption band energy bathochromically shifts to longer wavelengths in solvents with lower polarity (such as hexane and CH₂Cl₂) compared to those in more polar solvents (i.e CH₃CN and CH₃OH). This negative solvatochromic effect is a characteristic of a charge-transfer transition, in which the ground state is more polar than the excited state, and is in line with many of the platinum ĈN̂N or terpyridyl complexes reported in the literature.

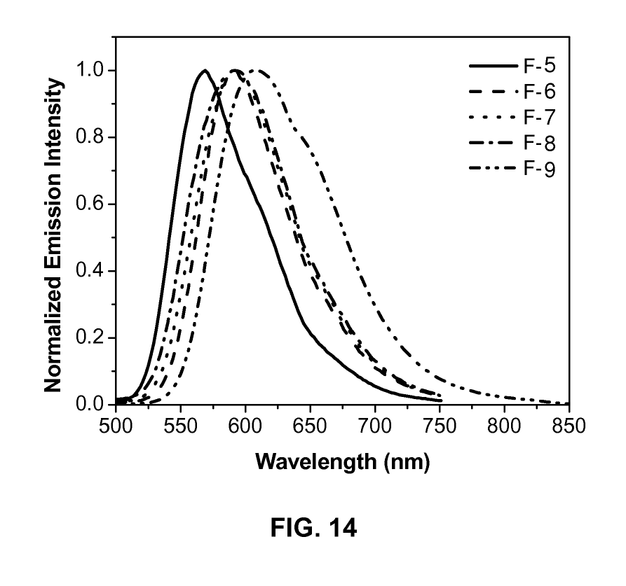

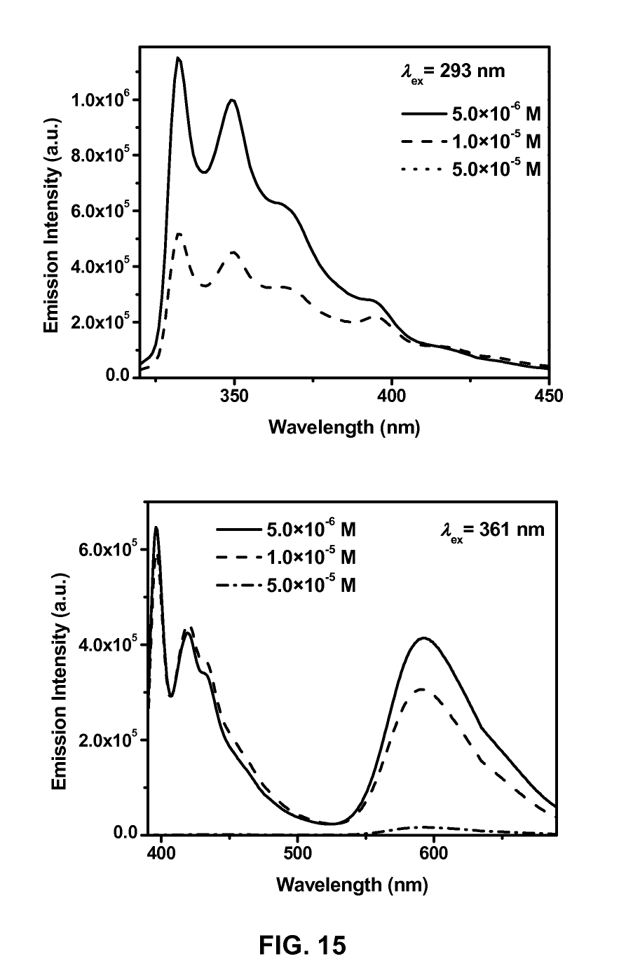

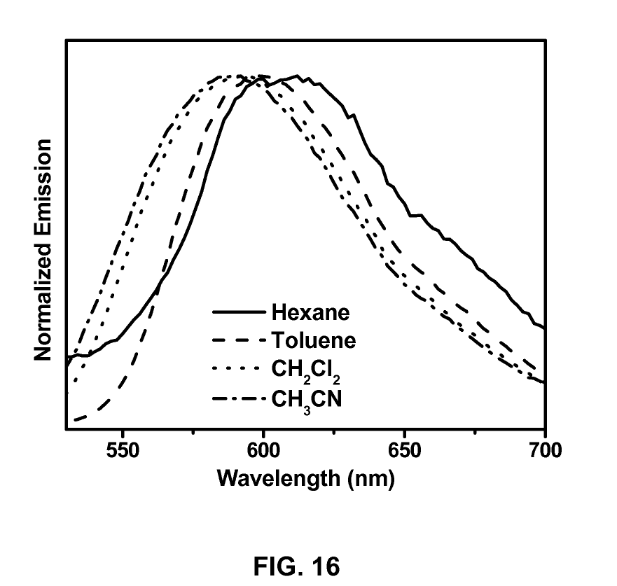

F-5-F-9 are emissive in solutions at room temperature and in glassy solutions at 77 K. FIG. 14 shows the emission spectra of F-5-F-9 in CH₂Cl₂ solution at a concentration of 5×10⁻⁵ mol/L at room temperature when excited at their respective charge transfer band. When excited at their ˜350 nm π,π* transition bands, F-5-F-8 give rise to the same red/orange emission band as that observed from excitation at their charge transfer bands. In contrast, F-9 exhibits a structured emission in the 320-500 nm regions in addition to the broad structureless emission band in the 500-750 nm regions (shown in FIG. 15) upon excitation at λₑₓ≦370 nm. The lifetimes of these two emission bands are quite distinct. The lifetime of the high-energy band is expected to be shorter than 5 ns and the lifetime of the orange emission band is hundreds of nanoseconds. In addition, the excitation spectra corresponding to these two emission bands are different. The excitation spectrum monitored at the high-energy band is consistent with the π,π* transition bands in the UV region. In contrast, the excitation spectrum measured for the orange emission corresponds to the charge transfer band in the UV-Vis absorption spectrum. Considering the different lifetimes, the distinct excitation spectra, and the different Stokes shifts, the high-energy emission from F-9 is tentatively attributed to fluorescence from the ¹π,π* state of the 2,7-diethynyl-9,9-dihexylfluorenyl bridging ligand; whereas the red/orange emission from F-5-F-9 is assigned as a triplet excited-state with charge transfer character, likely to be ³MLCT/³ILCT with reference to the other platinum ĈN̂N complexes reported in the literature. The charge transfer nature of the red/orange emission band could be supported by the fact that this band exhibits a negative solvatochromic effect. As shown in FIG. 16 for complex F-8, the emission energy decreases in less polar solvents such as hexane and toluene in comparison to those in polar solvents CH₂Cl₂ and CH₃CN. The same solvent effect was observed for the other four complexes as well.

The high-energy emission band observed in the solution of F-9 are not attributed to a trace amount of uncoordinated 4-fluorenyl-ĈN̂N ligand or fluorenyl impurity because the free 4-fluorenyl-ĈN̂N ligand exhibits a structureless fluorescence at 378 nm in CH₂Cl₂ upon excitation at 325 nm and the free fluorene shows a slightly structured fluorescence at 312 nm in CH₂Cl₂ upon excitation at 265 nm. Neither the emission energy nor the spectral feature from the free 4-fluorenyl-ĈN̂N ligand and free fluorene is consistent with the high-energy band observed in F-9 solution. However, this high-energy fluorescence band is substantially the same as the fluorescence spectrum of 2,7-diethynyl-9,9-dihexylfluorene. Though not wishing to be bound by a particular theory, the fact that this band is only observed from F-9 solution but not from the solutions of the other four complexes suggests that this band is associated with the unique structural feature of F-9, i.e. the presence of the 2,7-diethynyl-9,9-dihexylfluorenyl bridging ligand. This high-energy emission band in F-9 solution may emanate from the coordinated bridging ligand, rather than the free bridging ligand impurity because the excitation spectrum.

The possibility of the low-energy red/orange emission band arising from fluorescence resonance energy transfer (FRET) from the fluorenyl component to the Pt(ĈN̂N) component could be excluded based on the fact that no low-energy red/orange emission band appears in the emission spectrum of the free 4-fluorenyl-ĈN̂N ligand.

The concentration-dependent emission was investigated for all five complexes. When the concentration is increased from 5×10⁻⁶ mol/L to 5×10⁻⁴ mol/L, the emission energies and the shapes of the spectra of F-5-F-9 remain essentially the same, however, the emission intensity decreases at high concentrations and the emission lifetimes decrease with increased solution concentration. Because these complexes have almost no ground-state absorption at the emission band apex and the shapes of the spectra are the same, self absorption of emission could be excluded. As described above, the UV-vis absorption obeys the Lambert-Beer's law in the concentration range of 5×10⁻⁶ mol/L to 5×10⁻⁴ mol/L, suggesting that no ground-state aggregation occurs in this concentration range. Therefore, the intensity decrease at high concentrations cannot be attributed to the ground-state aggregation. In view of the decreased lifetime at high concentrations, it appears that self-quenching occurs at high concentrations. The self-quenching rate constants (k_{Q}) were measured according to the Stern-Volmer relation and the results are listed in Table 3. The k_{Q} for F-5-F-9 varies between 1.12×10⁹ and 1.63×10⁹ L mol⁻¹ s⁻¹, which falls in the same order of values reported for the terpyridine platinum phenylacetylide complexes (1.45×10⁹−1.74×10⁹ L mol⁻¹ s⁻¹). The self-quenching constant for F-5 was much higher than that for F-6. Though not wishing to be bound by a particular theory, this could be explained by the presence of the branched alkoxyl chain in F-6, which could reduce the π,π stacking and thus the formation of excimer.

TABLE 3
Photophysical parameters of F-5-F-9.

| | CH₂Cl₂^{a} | | | CH₃CN^{a} | | |
|---|---|---|---|---|---|---|
| | λ_{abs}^{c}/nm | λₑₘ^{c}/nm | k_{Q}^{d}/ | λₑₘ^{c}/nm | λ_{Tl-Tn}/nm (τ_{TA}/ns; | C₃H₇CN^{b} |
| | (ε/10³ L · | (τ₀/ns; | 10⁹ L · | (τₑₘ/ns, | ε_{Tl-Tn}/L · mol⁻¹ · | λₑₘ^{c}/nm |
| | mol⁻¹ · cm⁻¹) | Φₑₘ) | mol⁻¹ · s⁻¹ | Φₑₘ) | cm⁻¹; Φ_{T}) | (τₑₘ/μs) |
| F-5 | 282 (38.2), 312 (25.9), | 568 (960; | 1.63 | 566 (190; | 387 (140; 9070), | 548 (23.2), |
| | 336 (29.6), 354 (32.3), | 0.075) | | 0.067) | 656 (210; 4980; | 588 (23.9), |
| | 421 (8.6), 439 (8.8) | | | | 0.08) | 635 |
| F-6 | 291 (37.5), 323 (30.8), | 591 (950; | 1.12 | 588 (570; | 475 (620; 5500; | 542 (16.0), |
| | 354 (38.1), 419 (7.7), | 0.047) | | 0.042) | 0.16), 665 (680; | 586 (15.5), |
| | 439 (6.8) | | | | 4820), 800 (480) | |
| F-7 | 288 (37.7), 339 (35.7), | 593 (680; | 1.41 | 590 (510; | 400 (670; | 550 (14.0), |
| | 355 (30.8), 443 (9.2), | 0.073) | | 0.065) | 11480), 635 | 590 (14.2) |
| | 463 (8.8), 529 (1.0) | | | | (660; 3790; | |
| | | | | | 0.11) | |
| F-8 | 284 (48.2), 341 (31.9), | 593 (980; | 1.37 | 592 (758; | 400 (880; | 554 (14.0), |
| | 355 (30.9), 441 (10.3), | 0.076) | | 0.067) | 11000), 645 | 584 (14.5) |
| | 465 (9.2), 530 (1.2) | | | | (800; 1670; | |
| | | | | | 0.24) | |
| F-9 | 293 (80.4), 324 (79.6), | 602^{e} (890 | 1.94 | 607^{g} (825 | —^{j} | 588 (16.0), |
| | 361 (126.0), 441 | (97%), | | (94%), | | 629 (14.7) |
| | (24.4), 486 (24.6), 510 | 30 (3%)); | | 20 (6%)); | | |
| | (22.3) | 0.015) | | 0.008^{h}) | | |

^{a}Measured at room temperature.
^{b}In butyronitrile glassy solutions at 77 K.
^{c}At a concentration of 5 × 10⁻⁵ mol/L.
^{d}Self-quenching rate constant.
^{e}At a concentration of 5 × 10⁻⁶ mol/L.
^{g}At a concentration of 5 × 10⁻⁶ mol/L in acetone.
^{h}In acetone solution.
^{j}Too weak to be measured.

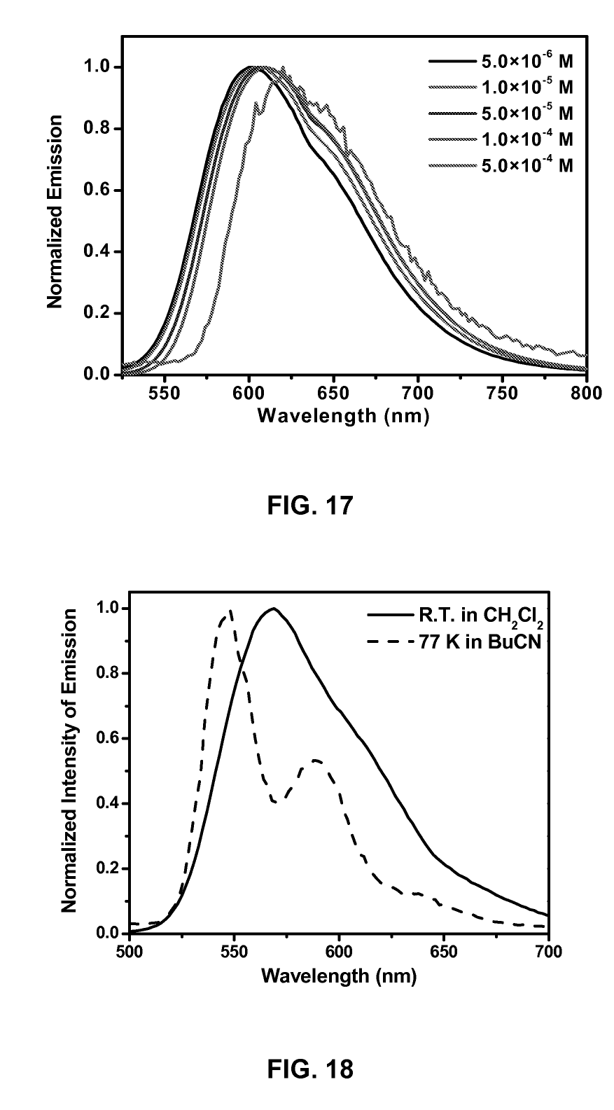

Complex F-9 exhibits a slightly different concentration-dependent behavior from the other four complexes. As shown in FIG. 17, the emission band maximum gradually red-shifts with increased concentration accompanied by reduced intensity and shortened lifetime. In view of the considerable ground-state absorption from 550 nm to 600 nm in F-9, it appears that the red-shift is induced by self absorption of the emission. Though not wishing to be bound by a particular theory, the reduced intensity and lifetime at high concentrations could be attributed to a combination of self-quenching, self absorption of emission and inner filter effect. The self-quenching constant is higher for F-9 than those for F-5-F-8, possibly due to the extended π-system that facilitates the formation of excimer. This notion could be partially supported by the bi-exponential decay of the emission for F-9 (shown in Table 3), in which the minor component may arise from an excimer emission. For the ¹π,π* emission at the UV region for F-9, it also exhibits significant intensity decrease when the concentration increases. This emission is completely quenched when the concentration reaches 5×10⁻⁵ mol/L or higher. Because of the intense absorption of F-9 in this spectral region, the reduced emission intensity should be caused predominately by self absorption of the emission, although the inner filter effect due to the intense absorption could also contribute.

With respect to the emission spectra shown in FIG. 14 for F-5-F-9, it appears that replacing the chloride co-ligand by the acetylide co-ligand reduces the emitting state energy, which has been seen in many reports for platinum ĈN̂N or terpyridyl complexes, and could be attributed to the π-donating ability of the acetylide ligand that would decrease the ³MLCT state energy. However, it is noted that the emission energy for F-6, F-7 and F-8 is quite similar, which may reflect the admixture of ³ILCT character that is independent on the nature of the co-ligand into the emitting state. The reduced emission energy of F-6 compared to that of F-5 is similar to that observed for the platinum ĈN̂N complexes without the fluorenyl substituent and could also be explained by the involvement of the ³ILCT character into the emitting state. The emission energies for all five complexes are quite similar to their respective platinum ĈN̂N congeners without the 4′-fluorenyl substituent. This feature is consistent with that observed from the UV-Vis absorption study, and indicates that the fluorenyl substituent likely twists from the plane of the Pt(ĈN̂N) component. Consequently, electronic interaction between the fluorenyl substituent and the ĈN̂N ligand is reduced. However, introducing the fluorenyl substituent on the ĈN̂N ligand pronouncedly enhances the emission efficiency and increases the emission lifetime for these complexes compared to their respective Pt complexes without the fluorenyl substituent, which is shown by F-5, F-6 and F-7.

F-5-F-8 are emissive in butyronitrile at 77 K. Compared to the emission at room temperature in CH₂Cl₂, the emission spectra shift to higher-energy and exhibit clear vibronic structures, as exemplified in FIG. 18 for F-5 and listed in Table 3 for the other complexes. The vibronic progression is in the range of 930 cm⁻¹ to 1390 cm⁻¹ for these complexes, which falls into the skeletal stretching modes of the ĈN̂N ligand. These spectral features are similar to those of the reported ĈNAN platinum acetylide complexes. The lifetimes of these complexes at 77 K is also comparable to those reported in the literature for other platinum ĈN̂N or terpyridyl complexes. However, the thermally induced Stokes shift for F-5 (580 cm⁻¹) and F-9 (395 cm⁻¹) is much smaller than those for F-6 (1440 cm⁻¹), F-7 (1230 cm⁻¹) and F-8 (1160 cm⁻¹). Considering the emission energy, the shape of the spectrum, the lifetime, and the thermally induced Stokes shift, the emitting state at 77 K is assigned as ³MLCT for F-6-F-8, and ³MLCT/³π,π* for F-5 and F-9.

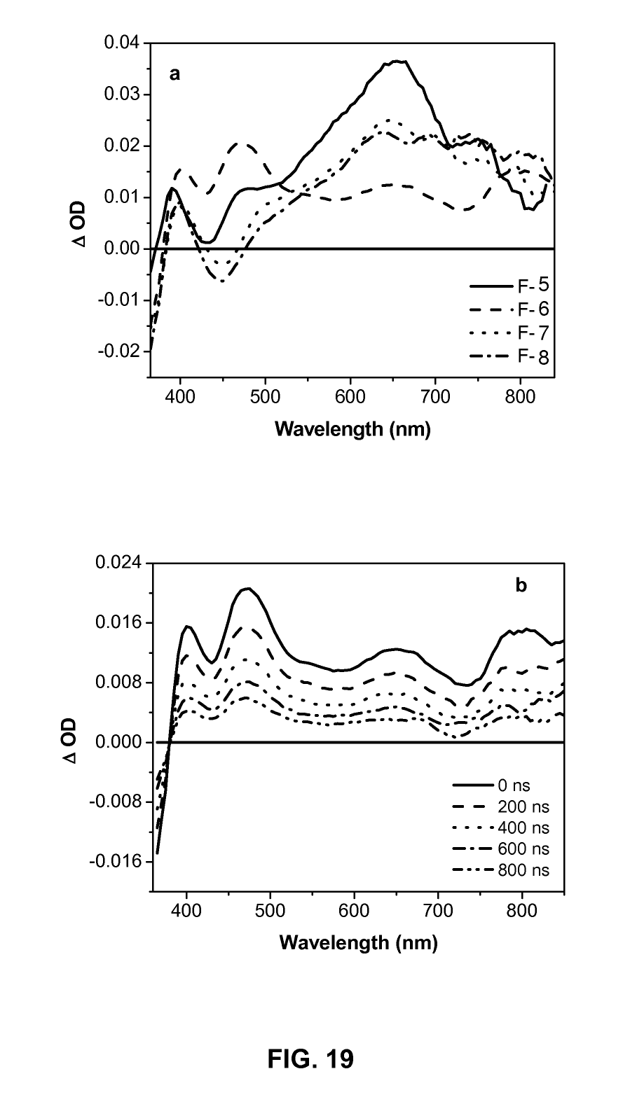

The lifetimes measured from the decay of the emission of F-5-F-9 suggest that the triplet excited state is much long-lived than the laser pulse width. Therefore, triplet excited-state absorption is expected to be observed for these complexes. FIG. 19 displays the triplet transient difference absorption (TA) spectra of F-5-F-8 in degassed CH₃CN solution at zero time-delay after the excitation. The time-resolved TA spectrum is exemplified in FIG. 19 for F-2 as well. The transient absorption from F-9 is too weak to be detected.

F-5 and F-6 exhibit broad positive absorption from 380 nm to 830 nm. The profiles of the TA spectra of F-7 and F-8 are similar to that of F-5, with the exception of that some bleaching occurred in the region of 420-480 nm and a flatter and stronger absorption in the NIR region of 770-850 nm. Though not wishing to be bound by a particular theory, the charge transfer band in the UV-vis absorption spectra and the different features in the NIR region indicate that the TA could possibly originate from the ³MLCT/³ILCT/³LLCT state for F-7 and F-8. For F-5 and F-6, the excited-state that gives rise to the transient absorption could be ³MLCT/³ILCT. All the TA decays monoexponentially throughout the whole spectral range monitored, indicating that the transient absorption arises from the same excited state or excited states in close proximity that are in equilibrium. In addition, the lifetime measured from the decay of the transient absorption and from the decay of the emission are essentially consistent, suggesting that and the TA may arise from the same excited state that emits, or a state that is in equilibrium with the emitting state.

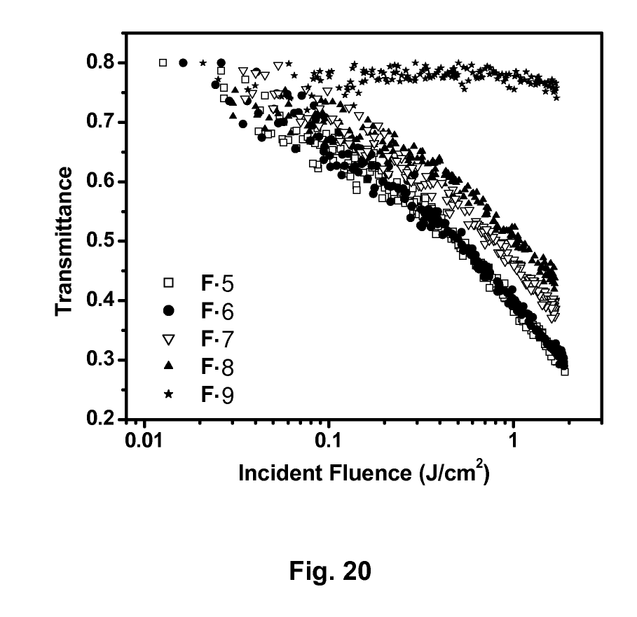

Complexes F-5-F-8 exhibit broad positive TA in most of the visible to the near-IR region, which implies that their excited-state absorption cross-sections are larger than the ground-state absorption cross-sections. In addition, the TA decay times are hundreds of nanoseconds. Therefore, reverse saturable absorption (RSA) could occur for these platinum complexes under the irradiation of ns laser pulses. To demonstrate this, the nonlinear transmission experiments were carried out for F-5-F-9 in CH₂Cl₂ and the results are illustrated in FIG. 20.

When the incident fluence is increased, the transmittance of F-5-F-8 decreases significantly. The RSA threshold, defined as the incident fluence at which the transmittance decreases to 70% of the linear transmittance, is 0.29 J/cm² for F-5 and F-6, 0.48 J/cm² for F-7, and 0.65 J/cm² for F-8. When the incident fluence is increased to 1.8 J/cm², the transmittance drops to 0.28 for F-5 and F-6, 0.37 for F-7, and 0.42 for F-8. For F-9, the transmittance almost keeps constant even at high fluences, indicating that almost no RSA occurs, which is consistent with the TA results discussed in the previous section. The strength of the RSA for these five complexes obviously follows this trend F-5=F-6>F-7>F-8>F-9.

The degree of RSA is determined at least in part by the ratio of the excited-state absorption cross-section to that of the ground-state. The ground-state absorption cross-sections at 532 nm for these complexes are 7.7×10⁻¹⁹ cm² for F-5, 5.4×10⁻¹⁹ cm² for F-6, 3.3×10⁻¹⁸ cm² for F-7, and 4.2×10⁻¹⁸ cm² for F-8 by using the E values obtained from their UV-Vis absorption spectra and the conversion equation σ=2303ε/N_{A}, where N_{A} is the Avogadro constant. To obtain the excited-state absorption cross-sections, open-aperture Z-scan experiments that measure the transmission changes due to nonlinear absorption were carried out at 532 nm using both ns and ps laser pulses for F-5-F-8. In the open-aperture Z-scan experiments, the laser beam at 532 nm is split by a wedge beamsplitter. One of the reflected beams is used to monitor the incident energy; while the transmitted beam is focused to the cuvette that contains the sample solution. The cuvette is placed on a translation stage that moves in the vicinity of the linear focal plane. The transmitted energy is monitored at each position where the translation stage stops. Then the transmission of the sample is plotted vs. the position of the translation stage. The resultant curve is fitted using the five-energy level model described previously to obtained the excited-state absorption cross sections. Li, Y.; Pritchett, T. M.; Huang, J.; Ke, M.; Shao, P.; Sun, W. J. Phys. Chem. A 2008, 112, 7200 (incorporated by reference herein).

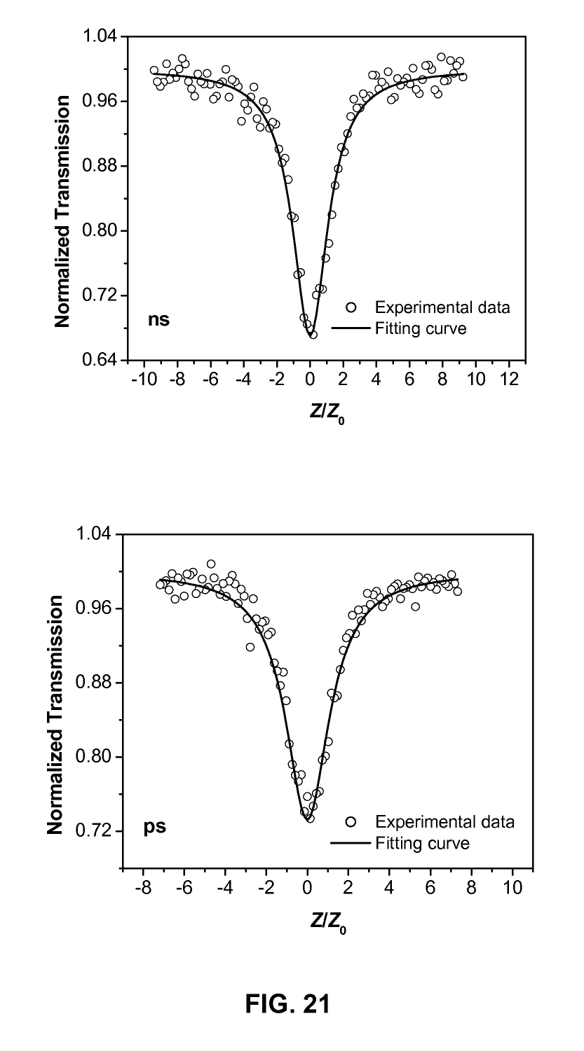

FIG. 21 shows the open-aperture Z-scan experimental data and the fitting curves for F-5. F-6-F-8 exhibit the similar results, while F-9 shows negligible nonlinear absorption. The transmission for F-5-F-8 decreases when the samples are moved close to the focal plane, i.e. the incident fluence increases, indicating the occurrence of reverse saturable absorption. By applying the ground-state absorption cross-sections determined from the UV-Vis absorption, the triplet and singlet excited-state lifetimes obtained from the decay of the respective ns and fs transient absorption (the τ_{T}'s are listed in Table 3, the τ_{S} is 12.1 ps for F-5, 6.9 ps for F-6, and 5.3 ps for F-7 and F-8), and the triplet excited-state quantum yields (listed in Table 3) to the five-band model, and using the procedure described above a single pair of excited-state absorption cross-section values (σₛ, σ_{T}) was obtained that simultaneously fit both the nanosecond and picosecond Z scans. These results are compiled in Table 4. For comparison purpose, Z scans of the corresponding (ĈN̂N)PtC₅H₇ complex has also been carried out, and the fitting results are listed in Table 4.

TABLE 4
Excited-state absorption cross-sections of F-5-F-8 in CH₂Cl₂ at 532 nm.

| | σ₀^{a} | σ_{S}^{b} | σ_{T}^{c} | | | |
|---|---|---|---|---|---|---|
| | (10⁻¹⁸ | (10⁻¹⁸ | (10⁻¹⁸ | | | |
| | cm²) | cm²) | cm²) | σ_{S}/σ₀ | σ_{T}/σ₀ | Φσ_{T}/σ₀ |
| F-5 | 0.765 | 62 ± 2 | 245 ± 5 | 81 | 320 | 25.6 |
| F-6 | 0.536 | 80 ± 3 | 103 ± 3 | 149 | 192 | 30.8 |
| F-7 | 3.29 | 130 ± 5 | 145 ± 5 | 40 | 44 | 4.8 |
| F-8 | 4.21 | 100 ± 5 | 75 ± 5 | 24 | 18 | 4.3 |
| (C\hat{ }N\hat{ }N)PtC₅H₇ | 1.60 | 19 ± 1 | 46 ± 2 | 12 | 29 | 14.7 |

^{a}Ground-state absorption cross-section.
^{b}Singlet excited-state absorption cross-section.
^{c}Triplet excited-state absorption cross-section.

The excited-state absorption cross-section values (both σₛ and σ_{T}) at 532 nm for F-5-F-8 are much larger than those for (ĈN̂N)PtC₅H₇ complex, which is in line with the trend observed in the UV-Vis absorption, reflecting the influence of fluorenyl unit on the ĈN̂N complexes. The ratios of σ_{S}/σ₀ and σ_{T}/σ₀ for F-5 and F-6 are among the largest values reported in the literature. When fitting the Z-scan results, the population fraction on related excited states are calculated versus time. For ns excitation, the triplet excited states (T₁ and T₂) are much more populated than the singlet excited states (S₁ and S₂). Therefore, the excited-state absorption for ns laser pulses is dominated by the triplet excited-state absorption. In such a case, the RSA is not only predominantly determined by the ratio of σ_{T}/σ₀, but also affected by the triplet excited state quantum yield. Taking these factors into account, the ratio of Φσ_{T}/σ₀ (listed in Table 4) was found to correlate very well with the observed RSA trend shown in FIG. 20. Therefore, in order to improve the RSA for ns laser pulses, the ratio of Φσ_{T}/σ₀ may be improved. In the case of complexes having similar excited-state absorption cross-section, the complex with the minimum ground-state absorption cross-section and higher triplet quantum yield would give rise to a larger ratio of Φσ_{T}/σ₀ and thus stronger RSA.

In sum, introducing the fluorenyl substituent on the ĈN̂N ligand causes a pronounced effect on the photophysics of mononuclear and dinuclear platinum(II) 6-phenyl-4-(9,9-dihexylfluoren-2-yl)-2,2′-bipyridine complexes F-5-F-9, which is reflected by their much larger molar extinction coefficients for their low-energy charge transfer absorption band, longer triplet excited-state lifetimes, higher emission quantum yields, and significantly increased ratios of the excited-state absorption cross-section to that of the ground-state compared to those of their corresponding Pt complexes without the fluorenyl substituent. Additionally, F-6 shows much improved solubility in CH₂Cl₂ (˜150 mg/mL), making it possible to prepare high-concentration solutions for two-photon absorption study in the near-IR region. Therefore, two-photon induced excited-state absorption was observed in the near-IR region for this complex.

### Example 4

### One-photon Photophysics and Two-photon Absorption of 4-[9,9-Di(2-ethylhexyl)-7-diphenylaminofluoren-2-yl]-2,2′:6′,2″-terpyridine and Their Platinum Chloride Complexes

All reagents and solvents (analytical grade) were purchased from VWR Scientific Company and used without further purification unless otherwise stated. The silica gel (230-400 mesh) was purchased from Alfa Aesar Company. Neutral Al₂O₃ (standard grade, 150 mesh) was purchased from Aldrich Company. All products were characterized by ¹H NMR, elemental analysis, and high resolution mass spectrometry (HRMS) ¹H NMR spectra were obtained using a Varian 400 MHz or 500 MHz VNMR spectrometer. HRMS was conducted on a Bruker Daltonics BioTOF system with electrospray ionization (ESI) source. Elemental analyses were conducted by NuMega Resonance Labs, Inc. in San Diego, Calif. High resolution MS data were obtained using Bruker BioT of III.

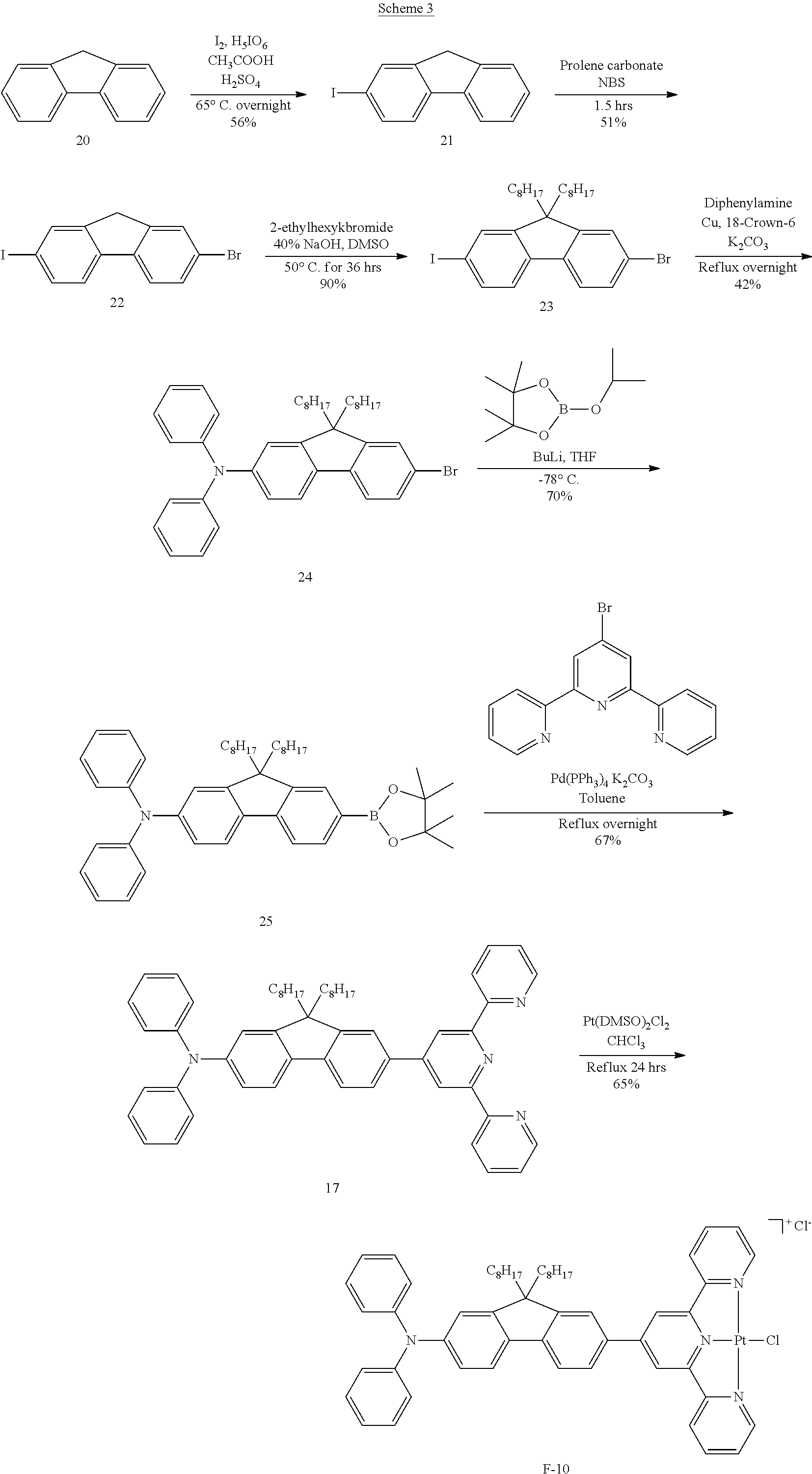

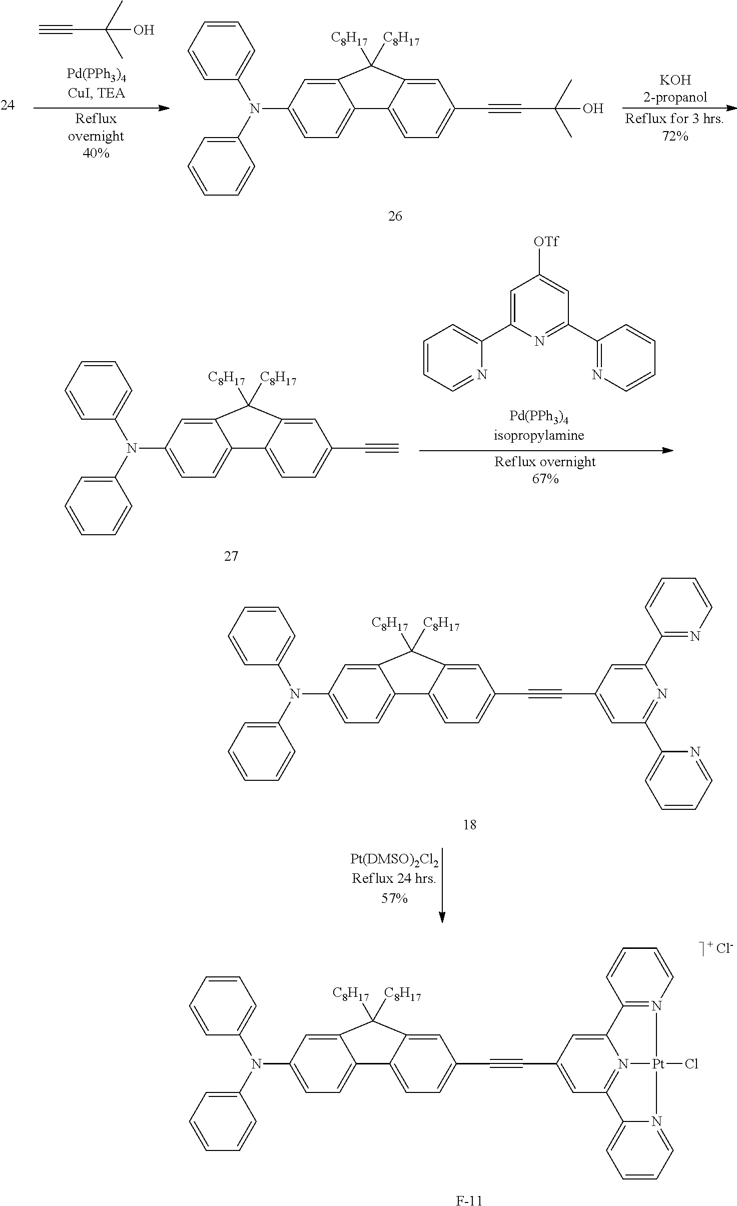

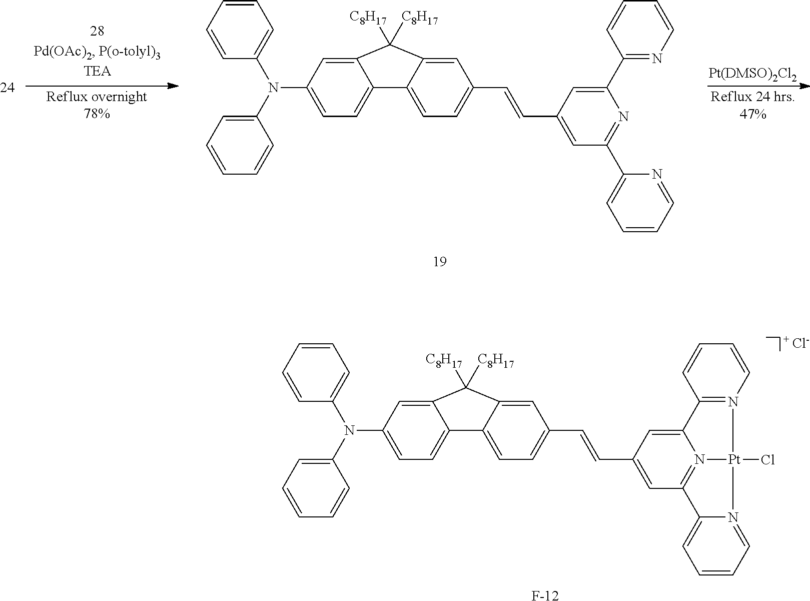

The precursors 2-iodofluorene (21), 2-bromo-7-iodofluorene (22), 2-bromo-9,9-di(2-ethylhexyl)-7-iodofluorene (23), 2-bromo-9,9-di(2-ethylhexyl)-7-diphenylaminofluorene (24), 4′-bromo-2,2′:6′,2″-terpyridine, 4′-vinyl-2,2′:6′,2″-terpyridine (28), 4′-(trifluoromethyl)sulfonyloxy-2,2′:6′,2″-terpyridine (OTf-tpy), were synthesized according to the procedures reported in the literature.

2-Bromo-9,9-di(2-ethylhexyl)-7-diphenylaminofluorene (24): 2-Bromo-9,9-di(2-ethylhexyl)-7-iodofluorene (23) (7.00 g, 0.012 mol), diphenylamine (2.58 g, 0.015 mol), 18-crown-6 ether (0.26 g, 0.001 mol), Cu (1.50 g, 0.023 mol), and K₂CO₃ (2.60 g, 0.019 mol) were added in 40 mL mesitylene. The mixture was heated to reflux under argon overnight. After the solvent was removed, the residue was dissolved in Et₂O. The organic phase was washed with water, and dried over MgSO₄. After removal of the solvent, the crude product was purified by a silica gel column using hexane as the eluent to give 4.50 g colorless oil (Yield: 42%). ¹H NMR (400 MHz, CDCl₃): δ 0.56 (8H, m), 0.75-1.00 (22H, m), 1.73-1.92 (4H, m), 7.00-7.12 (8H, m), 7.25 (4H, m), 7.46 (3H, m), 7.55 (1H, m) ppm.

7-(4,4,5,5-Tetramethyl-1,3,2-dioxaborolan-2-yl)-9,9-di(2-ethylhexyl)fluoren-2-yl-diphenylamine (25): Compound 24 (1.0 g, 1.12 mmol) was dissolved in 50 mL dried THF at −78° C. 1.6 M BuLi/hexane solution (2.0 mL, 3.20 mmol) was added slowly. The mixture was stirred at −78° C. for 1 hr. 2-Isopropoxy-4,4,5,5-tetramethyl-1,3,2-dioxaborolane (0.42 mL, 1.12 mmol) was added dropwise. The mixture was allowed to reach room temperature and stirred at r.t. overnight. After that 50 mL brine was added to terminate the reaction. The aqueous layer was extracted with Et₂O for three times (30 mL each time). The combined organic layer was dried over Na₂SO₄. After removal of the solvent, the crude product was purified by a silica gel column using hexane:toluene (v/v=5:1) as the eluent to give 735 mg colorless oil (Yield: 70%). ¹H NMR (400 MHz, CDCl₃): δ 0.50 (8H, m), 0.6-0.9 (22H, m), 1.34 (12H, s), 1.7-2.0 (4H, m) 6.9-7.1 (8H, m), 7.21 (4H, m), 7.58 (2H, m) 7.76 (2H, m) ppm.

Ligand 17: Compound 25 (0.75 g, 0.80 mmol) and 4′-bromo-2,2′:6′,2″-terpyridine (311 mg, 1.00 mmol) were added to 50 mL toluene. 2 M K₂CO₃ aqueous solution (2.5 mL) was added. The mixture was degassed in argon for 20 mins. Pd(PPh₃)₄ (46 mg, 0.04 mmol) was added. The mixture was heated to reflux under argon overnight. The organic layer was washed with water, dried over MgSO₄ and filtered. After the solvent was removed, the crude product was purified on a silica gel column eluted by CH₂Cl₂ to give 422 mg pale yellow solid (Yield: 67%). ¹H NMR (500 MHz, CDCl₃): δ 0.6 (8H, m), 0.7-1.0 (22H, m), 1.86-2.10 (4H, m), 7.03 (2H, m), 7.11 (6H, m), 7.16 (4H, m), 7.38 (2H, t, J=6.5 Hz), 7.66 (1H, m), 7.76 (1H, m), 7.86 (2H, t, J=7.0 Hz), 7.91 (2H, t, J=7.0 Hz), 8.70 (2H, d, J=8.0 Hz), 8.78 (4H, m) ppm. HRMS: calcd for [C₅₆H₆₁N₄]⁺, 789.4891; found, 789.4875. (100%).

Complex F-10: Ligand 17 (560 mg, 0.71 mmol) and Pt(DMSO)₂Cl₂ (300 mg, 0.73 mmol) were added to 80 mL CHCl₃. The mixture was heated to reflux under argon for 24 hrs. After the solvent was removed, the residue was purified on an Al₂O₃ column eluted by CH₂Cl₂, followed by a mixture of CH₂Cl₂ and MeOH (1:1). The crude product was purified by recrystallization from CH₂Cl₂/Hexane/Et₂O to give 486 mg red solid (Yield: 65%). ¹H NMR (500 MHz, CDCl₃): δ 0.6 (8H), 0.7-1.2 (22H), 2.0 (2H, m), 2.35 (2H, m), 7.13 (8H, m), 7.29 (4H, m), 7.48 (2H, m), 7.70 (1H, d, J=8.0 Hz), 7.97 (1H, d, J=8.0 Hz), 8.32 (3H, m), 8.53 (3H, m), 8.77 (2H, t, J=8.0 Hz), 9.11 (2H, d, J=7.5 Hz) ppm. HRMS: calcd for [C₅₆H₆₀N₄PtCl]⁺, 1019.4156; found, 1019.4159. (100%). Anal. Calcd. for C₅₆H₆₀N₄PtCl₂.2.5CH₂Cl₂: C, 55.30; H, 5.12; N, 4.41. Found: C, 55.75; H, 5.30; N, 4.57.

Compound 26: Compound 24 (1.10 g, 1.70 mmol) and 2-methyl-3-butyn-2-ol (0.33 mL, 3.40 mmol) was added to 30 mL triethylamine. CuI (7.00 mg, 0.04 mmol), PPh₃ (18.00 mg, 0.07 mmol), and Pd(PPh₃)₄ (50.0 mg, 0.04 mmol) were added. The mixture was heated to reflux under argon overnight. The solvent was removed, and the residue was dissolved in CH₂Cl₂. The solution was washed with water, and dried over Na₂SO₄. After the solvent was removed, the crude product was purified on a silica gel column eluted by a mixture of hexane and CH₂Cl₂ (v/v=1:1) to give 435 mg colorless oil (Yield: 40%). ¹H NMR (400 MHz, CDCl₃): δ 0.46-0.53 (8H, m), 0.68-0.86 (22H, m), 1.62 (6H, t, J=2.8 Hz), 1.75-1.89 (4H, m), 1.99 (1H, t, J=3.2 Hz), 7.01 (8H, m), 7.22 (4H, m), 7.34 (2H, m), 7.52 (2H, m) ppm.

Compound 27: Compound 26 (0.41 g, 0.64 mmol) and KOH (0.30 g, 5.36 mmol) were added to 10 mL 2-propanol. The mixture was heated to reflux under argon for 3 hrs. After the solvent was removed, the residue was purified on a silica gel column eluted by hexane to give 268 mg colorless oil (Yield: 72%). ¹H NMR (400 MHz, CDCl₃): δ 0.51 (8H, m), 0.68-0.90 (22H, m), 1.55-1.99 (4H), 3.06 (1H, s), 6.96-7.06 (8H, m), 7.23 (4H, m), 7.42 (2H, m), 7.53 (2H, m) ppm. HRMS: calcd for [C₄₃H₅₁N]⁺, 581.4016; found, 581.4019 (100%).

Ligand 18: Compound 27 (323 mg, 0.56 mmol) and OTf-tpy (212 mg, 0.56 mmol) were added to a mixture of benzene (50 mL) and iso-propylamine (20 mL). Pd(PPh₃)₄ (30 mg, 0.02 mmol) was then added. The mixture was heated to reflux under argon for overnight. After the solvent was removed, the residue was dissolved in CH₂Cl₂. The solution was washed with water, and dried over Na₂SO₄. After the solvent was removed, the crude product was purified on a neutral Al₂O₃ column eluted by a mixture of hexane and CH₂Cl₂ (v/v=10/1) to remove the unreacted reagent, and then eluted by a mixture of hexane and CH₂Cl₂ (1:1) to give 341 mg pale yellow solid (Yield: 75%). ¹H NMR (400 MHz, CDCl₃): δ 0.58 (8H, m), 0.7-1.0 (22H), 1.8-2.0 (4H, m), 7.0 (8H, m), 7.23 (4H, t, J=8.1 Hz), 7.35 (2H, m), 7.57 (4H, m), 7.87 (2H, dt, J=7.5, 1.5 Hz), 8.63 (4H, m), 8.74 (2H, dt, J=4.8 Hz) ppm. HRMS: calcd for [C₅₈H₆₁N₄]⁺, 813.4891; found, 813.4917 (100%).

Complex F-11: Ligand 18 (340 mg, 0.42 mmol) and Pt(DMSO)₂Cl₂ (212 mg, 0.52 mmol) were added to 80 mL CHCl₃. The mixture was heated to reflux under argon for 24 hrs. After the solvent was removed, the residue was purified by an Al₂O₃ column eluted first by a mixture of CH₂Cl₂ and CH₃CN (v/v=5/2), and then by a mixture of CH₂Cl₂ and MeOH (10:1). The crude product was further purified by recrystallization from CH₂Cl₂/Hexane/Et₂O to give 258 mg red solid (Yield: 57%). ¹H NMR (400 MHz, CDCl₃): δ 0.6 (8H), 0.7-1.0 (22H), 1.8-2.0 (4H), 7.10 (8H, m), 7.26 (4H, m), 7.52 (1H, m), 7.60 (2H, m), 7.70 (2H, m), 8.20 (2H, m), 8.40 (2H, t, J=8.0 Hz), 8.87 (2H, d, J=8.0 Hz), 9.01 (2H, d, J=4.8 Hz), 9.29 (2H, s) ppm. HRMS: calcd for [C₅₈H₆₀N₄PtCl+C₂H₆O+H]⁺, 1089.4576; found, 1089.4543 (100%). Anal. Calcd. for C₅₈H₆₀N₄PtCl₂.0.8CH₂Cl₂.CH₃CH₂OH: C, 61.22; H, 6.04; N, 4.97. Found: C, 61.21; H, 5.71; N, 4.70.

Ligand 19: Compound 24 (580 g, 0.90 mmol), 28 (233 mg, 0.90 mmol) and P(o-tolyl)₃ (100 mg, 0.33 mmol) were added to 30 mL triethylamine. Pd(OAc)₂ (10 mg, 0.04 mmol) was then added. The mixture was heated to reflux under argon overnight. The mixture was filtered out and washed with Et₂O. The filtrate was washed with water. The organic phase was dried over Na₂SO₄. The crude product was purified by a neutral Al₂O₃ column eluted by CH₂Cl₂ to give 571 mg orange viscous oil (Yield: 78%). ¹H NMR (500 MHz, CDCl₃): δ 0.6 (8H, m), 0.7-1.0 (22H), 1.8-2.1 (4H), 7.15 (9H, m), 7.25 (4H, m), 7.40 (2H, dd, J=6.5, 1.5 Hz), 7.59 (2H, m), 7.67 (2H, d, J=7.5 Hz), 7.93 (2H, d, J=16 Hz), 7.92 (2H, t, J=7.5 Hz), 8.57 (2H, s), 8.72 (2H, d, J=7.5 Hz), 8.80 (2H, d, J=5.0 Hz) ppm. HRMS: calcd for [C₅₈H₆₃N₄]⁺, 815.5047; found, 815.5015. (100%) Anal. Calcd. for C₅₈H₆₂N₄.toluene.1.5H₂O: C, 83.56; H, 7.88; N, 6.00. Found: C, 83.11; H, 7.84; N, 6.02.

Complex F-12: Compound 19 (91.8 mg, 0.11 mmol) and Pt(DMSO)₂Cl₂ (47.6 mg, 0.12 mmol) were added to 80 mL CHCl₃. The mixture was heated to reflux under argon for 24 hrs. After the solvent was removed, the residue was purified by an Al₂O₃ column eluted by CH₂Cl₂, and then by 1:1 CH₂Cl₂ and MeOH. The crude product was purified by recrystallization from CH₂Cl₂/Hexane/Et₂O to give 55.8 mg red solid (Yield: 47%). ¹H NMR (400 MHz, CDCl₃): δ 0.6 (8H, m), 0.7-1.0 (22H), 1.8-2.2 (4H), 7.10 (8H, m), 7.26 (4H, m), 7.49 (2H, m), 7.61 (2H, m), 7.85 (2H, m), 8.20 (2H, m), 8.50 (5H, m), 8.92 (2H, m) ppm. HRMS: calcd for [C₅₈H₆₂N₄PtCl]⁺, 1045.4313; found, 1045.4339 (100%). Anal. Calcd. for C₅₈H₆₂N₄PtCl₂.2.5CH₂Cl₂: C, 56.18; H, 5.22; N, 4.33. Found: C, 56.09; H, 5.32; N, 4.66.

### Photophysical Measurements

The electronic absorption spectra were recorded on a SHIMADZU 2501 PC UV-vis spectrophotometer. The emission spectra at room temperature were recorded on a SPEX Fluorolog-3 fluorometer/phosphorometer. Complexes F-10, F-11, and F-12 were dissolved in CH₃CN, and ligands 17, 18, and 19 were dissolved in CH₂Cl₂. The solutions were degassed via bubbling Ar gas for 30 min prior to each measurement. The emission lifetimes of ligand 17, 18, and 19 were measured on an Edinburgh LP920 laser flash photolysis spectrometer. The excitation beam was the third-harmonic output (355 nm) of a Quantel Brilliant Q-switched Nd:YAG laser (FWHM pulsewidth was 4.1 ns and the repetition rate was set at 1 Hz). The sample solutions were degassed for 30 min. before each measurement. The emission lifetime of complexes F-10, F-11, and F-12 were measured by time correlated single photon counting (TCSPC) technique (λₑₓ=375 nm). The sample solutions were prepared to have an absorbance at 375 nm of approximately 0.1-0.2. The emission quantum yields of the ligands 17, 18, and 19, and the complexes F-10, F-11, and F-12 were determined by the comparative method, in which 9,10-dipenylanthracence in ethanol (φₑₘ=0.9, excited at 350 nm) was used as the reference for the ligands, and a degassed aqueous solution of [Ru(bpy)₃]Cl₂ (φₑₘ=0.042, excited at 436 nm) was used as the reference for the complexes. The nanosecond triplet transient difference absorption spectra were measured on the Edinburgh LP920 laser flash photolysis spectrometer. The excitation was provided by the third-harmonic output (355 nm) of the Quantel Brilliant Q-switched Nd:YAG laser. The solutions were degassed via bubbling Ar gas for 30 min. before each measurement. The absorbance of the solution was adjusted to A=0.4 at 355 nm in a 1-cm quartz cuvette. The femtosecond transient difference absorption spectra were measured on a femtosecond pump-probe UV-vis spectrometer (HELIOS) manufactured by Ultrafast Systems LLC. The sample was excited at 400 nm with a 260 fs Ti:sapphire laser pulse, and the absorption was probed from 440 to 800 nm with white light continuum.

### Two Photon Induced Fluorescence Spectroscopic Measurement

The 2PA spectra of the ligands 17-19 in toluene (toluene was chosen as the solvent instead of CH₂Cl₂ because the toluene solutions of 17-19 were much stable than the CH₂Cl₂ solutions upon laser irradiation) were measured by modified fluorescent method. The measurements were done by monitoring the wavelength-dependent two-photon-excited fluorescence, which allowed for direct measurement of 2PA in a broad variety of compounds with the fluorescence (or phosphorescence) quantum yield Φ>0.005. The relative spectrum was measured using coumarin 485 in methanol as a reference with a 2-nm interval for both the sample and the reference. Then the spectrum was normalized to a correct cross section measured at a single wavelength relative to 9,10-dichloroanthracene in dichloromethane.

The 2PA experimental setup and the detailed description of the experimental method were reported previously. N. S. Makarov, M. Drobizhev, A. Rebane, Opt. Expr. 2008, 16, 4029-4047 (incorporated by reference herein). The laser system comprises Ti:Sapphire femtosecond oscillator (Coherent Mira 900) pumped with a CW frequency-doubled Nd:YAG laser (Coherent Verdi). The oscillator is used to seed a 1-kHz repetition rate Ti:Sapphire femtosecond regenerative amplifier (Coherent Legend-HE). The output pulses from the amplifier are down-converted with an optical parametric amplifier (OPA) (Quantronix TOPAS-C). The output of the OPA (signal and idler) can be continuously tuned from 1100 to 2200 nm. For two-photon excitation second harmonic of either idler (790-1100 nm) or signal (550-790 nm) beam was used. A Glan-prism polarizer was placed before the second harmonic generation (SHG) crystal to select either vertical (signal) or horizontal (idler) polarization. The residual fundamental beam (signal or idler) was cut with color filters, placed after the SHG crystal. For one-photon excitation, the second harmonic of the Ti:Sapphire amplifier output (397 nm) was used. The polarization of the excitation laser beam was vertical for both 1PA and 2PA. In the case of second harmonic of signal, a λ/2 plate was used after the reference detector to rotate polarization by 90°.

The fluorescence was collected at 90° to the laser beam direction with a spherical mirror (f=50 cm, diameter d=10 cm), which focused the horizontally-elongated image of fluorescence track with the magnification ratio ˜1:1 on the entrance plane of the fluorescence grating spectrometer (Jobin Yvon Triax 550). The height of the vertical spectrometer slit was much larger than the height of the fluorescence image. The spectral dispersion on a two-dimensional CCD detector (Jobin Yvon Spectrum One) occurred in the horizontal direction, while the signal in the vertical direction was integrated over the whole slit height. The slit width was much smaller than the horizontal dimension of the fluorescence image and was kept the same in both 1PA and 2PA signal measurements. While recording the fluorescence spectrum, special care was taken to eliminate any spurious signals, such as scattered laser light, fluorescence of impurities, etc. The fluorescence spectra of the sample excited via 1PA and 2PA always had the same shape. The fluorescence intensity was measured by integrating the CCD output over 0.5-5 seconds and over 40-60 nm spectral region around the emission peak wavelength. Each data point was obtained by averaging of 2-5 acquisitions.

The raw spectra were obtained by measuring 2PA-excited fluorescence normalized to a square of the excitation laser power in a range of interest of excitation wavelengths. The absolute spectra were obtained by calibrating the unknown efficiency of fluorescence registration and fluorescence quantum yield and by correcting the raw spectra for the wavelength-dependent spatial and temporal laser profile.

### Z-Scan Measurement and Fitting

An optical parametric generator (EKSPLA PG401) pumped by the third harmonic output of an EKSPLA PL2143A passively mode-locked, Q-switched Nd:YAG laser (pulsewidth=21 ps, repetition rate=10 Hz) was used as the light source. A 25-cm or 30-cm plano-convex lens was used to focus the beam to a beam waist of ˜30 μm at the focal point, which gave rise to a Rayleigh length (

$${z}_{0} = {πω}_{0}^{2} / λ ,$$

where ω₀ is the radius at the beam waist) of approximately 3.4-4.9 mm at the spectral range used for the Z-scan study. Therefore, the sample solution placed in a 2-mm cuvette could be considered as thin samples. A 50-cm plano-convex lens was placed at approximately 30 cm after the linear focal plane to collect all of the transmitted light into the Molectron J4-09 joulemeter probe.

The experimental Z-scan data were fitted using the five-band model, in which each chromophore molecule is assumed to lie in the vibration-rotation manifold of one of five electronic states: the ground state, S₀, a singlet; one of two singlet excited states, S₁ or S₂; or one of two triplet states, T₁ or T₂. The following rate equations specify the time evolution of n₀, n_{S}, n_{T}, n_{S2}, and n_{T2}, the number densities of molecules in, respectively, the S₀-, S₁-, T₁-, S₂—, and T₂-bands.

$$\begin{matrix}\frac{\partial {n}_{0}}{\partial t} = - \frac{{σ}_{0}}{hv} {n}_{0} I - \frac{{σ}_{2}}{2 hv} {n}_{0} {I}^{2} + {k}_{S} {n}_{S} + {k}_{T} {n}_{T} & ( 1 ) \\ \frac{\partial {n}_{S}}{\partial t} = \frac{{σ}_{0}}{hv} {n}_{0} I + \frac{{σ}_{2}}{2 hv} {n}_{0} {I}^{2} - ( {k}_{S} + {k}_{isc} ) {n}_{S} - \frac{{σ}_{S}}{hv} {n}_{S} I + {k}_{S 2} {n}_{S 2} & ( 2 ) \\ \frac{\partial {n}_{T}}{\partial t} = {k}_{isc} {n}_{S} - {k}_{T} {n}_{T} - \frac{{σ}_{T}}{hv} {n}_{T} I + {k}_{T 2} {n}_{T 2} & ( 3 ) \\ \frac{\partial {n}_{S 2}}{\partial t} = \frac{{σ}_{S}}{hv} {n}_{S} I - {k}_{S 2} {n}_{S 2} & ( 4 ) \\ {n}_{T 2} = N - {n}_{0} - {n}_{S} - {n}_{T} - {n}_{S 2} & ( 5 )\end{matrix}$$

Here, vis the frequency of the laser radiation, h is the Planck constant, I is the irradiance, and N is the overall number density of chromophore molecules. The constants k_{S}, k_{S2}, k_{T}, k_{T2}, and k_{isc} are, respectively, the rate constants for the decays S₁→S₀, S₂→S₁, T₁→S₀, T₂→T₁, and S₁→T₁. Equations (1) and (2), which reflect the effects of radiative transitions from the ground state, include terms for both single-photon and two-photon S₀→S₁ transitions: σ₀n₀I[hv]⁻¹ and σ₂n₀I^{2[}2hv]⁻¹, respectively. At wavelengths at which the linear absorption of the material is non-negligible (here, for wavelengths less than 680 nm), the former process dominates and σ₂(λ) is set to zero. Conversely, at wavelengths longer than 740 nm, S₁ is assumed to be populated from the ground state primarily by two-photon absorption and σ₀(λ) is set to zero in the fitting.

Completing the five-band model is the following extinction law, which describes the decrease in intensity of the propagating beam as a result of single-photon absorption from S₁ and T₁ and either single- or two-photon absorption from S₀, depending on the wavelength.

$$\frac{\partial I}{\partial z} = - ( {σ}_{0} {n}_{0} + {σ}_{S} {n}_{S} + {σ}_{T} {n}_{T} ) I - \frac{{σ}_{2}}{hv} {n}_{0} {I}^{2} . \qquad ( 6 )$$

### Results

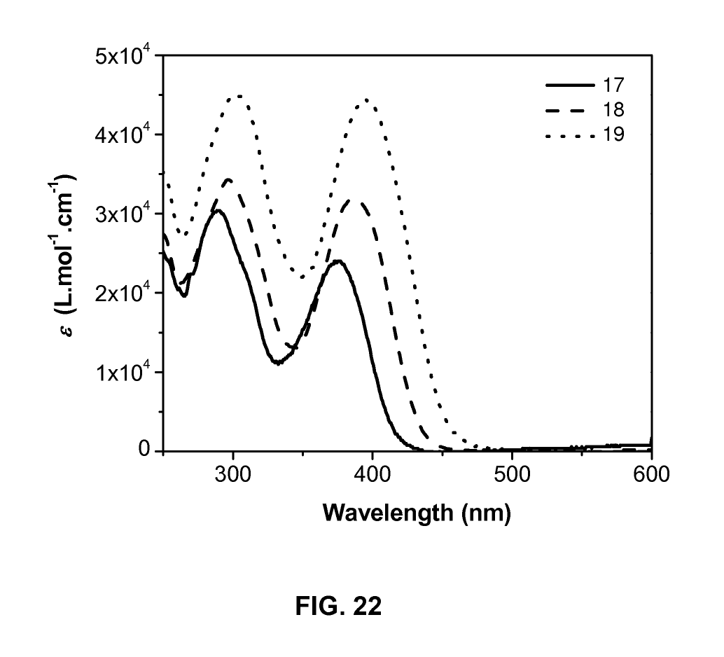

Electronic absorption: The electronic absorption spectra of 17-19 in CH₂Cl₂ at a concentration of 1×10⁻⁵ mol/L are shown in FIG. 22 and the molar extinction coefficients are summarized in Table 5. 17-19 all exhibit intense absorption in the UV and blue regions, which can be assigned as ¹π,π* and ¹π,π*/¹ICT (intramolecular charge transfer) transitions, respectively. It is obvious that going from 17 to 19, the energies of the absorption bands decrease, accompanied by an increase of the molar extinction coefficients. This trend could be attributed to the enhanced electronic coupling between the diphenylaminofluorene component and the terpyridine component, which results in the more extended 7r-conjugation and bathochromic shift of the absorption bands. The assignment of ¹π,π* transitions to these absorption bands is supported by the large extinction coefficients of these bands and by the solvent-dependency study. Minor solvent effect was observed for 17, which is consistent with the ¹90 ,π* character. The possible mixture of the ¹ICT character into the blue absorption band could be rationalized by the electron-donating ability of the diphenylamino substituent and the electron-withdrawing ability of the terpyridine component. It is further supported by the acid-titration study that will be discussed in the following paragraph, in which the increased electron-withdrawing ability of the protonated terpyridine causes a red-shift of the ¹ICT band, whereas the π,π* transition remains the same energy. Similar phenomenon was observed for 18 and 19.

TABLE 5
Electronic absorption and emission data for 17, 18, 19, F-10, F-11, and F-12.

| | λ_{abs}/nm | λₑₘ/nm (τ) | Φₑₘ | λₑₘ/nm (τ/μs) |
|---|---|---|---|---|
| | (ε/10⁴ L · mol⁻¹ · cm⁻¹)^{[a]} | R.T. | R.T. | 77 K |
| 17 | 290 (2.96), 376 (2.32) | 484 (12 ns (26%), 102 ns (74%))^{[b]} | 0.59^{[d]} | 522, 569^{[f]} |
| 18 | 297 (3.48), 387 (3.16) | 505 (12 ns (23%), 91 ns (76%))^{[b]} | 0.70^{[d]} | 582^{[f]} |
| 19 | 304 (4.08), 394 (4.44) | 526 (33 ns (41%), 112 ns (59%))^{[b]} | 0.42^{[d]} | 594^{[f]} |
| F- | 284 (4.03), 334 (3.63), | 515 (48 ps (29%), 2241 ps (71%))^{[c]} | 0.00047^{[e]} | 566 (119)^{[g]} |
| 10 | 471 (1.75) | | | |
| F- | 283 (3.64), 338 (3.49), | 486 (86 ps (2%), 3686 ps (98%))^{[c]} | 0.00035^{[e]} | 602 (88)^{[g]} |
| 11 | 361 (3.63), 466 (2.06) | | | |
| F- | 284 (4.16), 338 (3.55), | 532 (150 ps (8%), 2976 ps (92%))^{[c]} | 0.00019^{[e]} | 644 (22)^{[g]} |
| 12 | 365 (3.61), 492 (3.04) | | | |

^{[a]}Electronic absorption band maxima and molar extinction coefficients in CH₂Cl₂ for 17-19, and in CH₃CN for F-10-F-12, c ≈ 1 × 10⁻⁵ mol/L.
^{[b]}Room temperature emission band maxima and decay lifetimes measured at a concentration of 1 × 10⁻⁵ mol/L.
^{[c]}The emission band maxima and decay lifetimes measured in CH₃CN solutions. λₑₓ = 375 nm.
^{[d]}Emission quantum yield measured in CH₂Cl₂ solutions with A₃₅₀ = 0.1.
^{[e]}Emission quantum yield measured at λₑₓ = 436 nm with A₄₃₆ = 0.1 in CH₃CN solution. [Ru(bpy)₃]Cl₂ was used as the reference.
^{[f]}The emission band maxima at 77 K measured with 10 equivalents of CH₃I in 4:1 CH₃CH₂OH/CH₃OH glassy solution, c ≈ 1 × 10⁻⁵ mol/L.
^{[g]}The emission band maxima and decay lifetimes at 77 K measured in BuCN glassy solution, c ≈ 1 × 10⁻⁵ mol/L. λₑₓ = 355 nm.

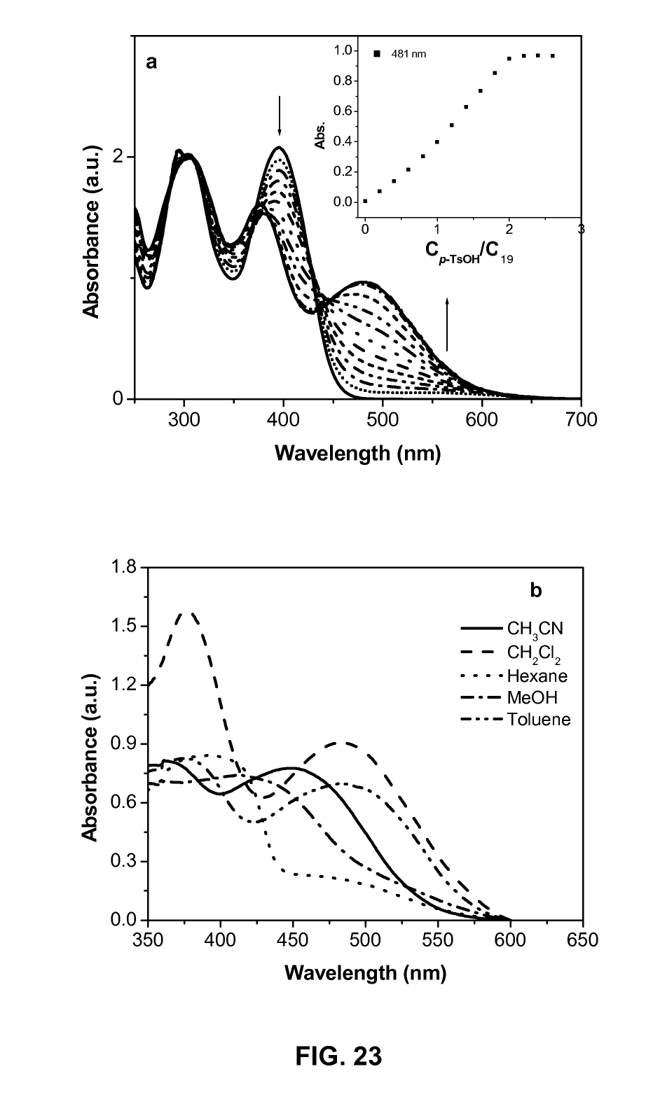

Because diphenylamino group is an electron-donating group and terpyridine motif would become a stronger electron-withdrawing group upon protonation, therefore, stronger and red-shifted intramolecular charge transfer (ICT) is anticipated to occur in acidic solution. To verify this, titration of 17-19 with p-TsOH was carried out. As exemplified in FIG. 23 for 19, upon addition of p-TsOH, the absorption band at ca. 394 nm decreases, accompanied by the increase of a new absorption band at ca. 481 nm. These changes could be attributed to the protonation of the nitrogens in terpyridine. The resultant positive charges facilitate electron transfer from the electron-rich diphenylamino group to the electron-deficient protonated terpyridine motif, which increases the degree of intramolecular charge transfer ('ICT) transition and bathochromically shifts the ¹ICT band. In this case, the ¹π,π* transition and the ¹ICT transition are separated, which results in the decrease of the original ¹π,π*/¹ICT absorption band at 394 nm. The charge transfer nature of the new absorption band at ca. 481 nm is evident from the negative solvatochromic effect due to the more polar charge-separated ground-state and the less polar excited state after charge transfer. As shown in FIG. 23b for the protonated ligand 19, the low-energy absorption band is blue-shifted in more polar solvents, such as in MeOH and CH₃CN, compared to those in less polar solvents, which is a characteristic of a more polar charge-separated ground state. Similar phenomena were observed for 17 and 18.

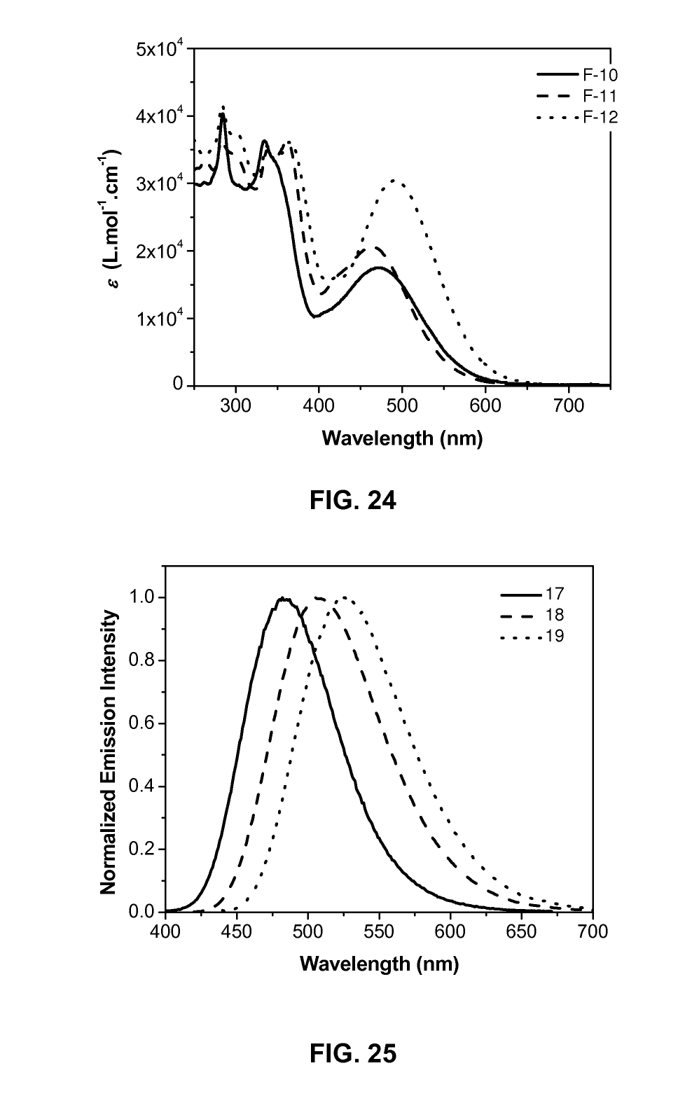

The electronic absorption spectra of platinum complexes F-10, F-11, and F-12 were measured in CH₃CN solutions. As shown in FIG. 24 and summarized in Table 5, these complexes exhibit intense absorption bands below 370 nm, which are assigned as the π,π* transitions from the ligand. In addition, all complexes exhibit a broad intense absorption band in the visible region between 400 and 650 nm. The energies of these absorption bands are similar to those observed from the protonated ligands, implying that these bands possess an intraligand charge transfer (¹ILCT) character from the diphenylamino component to the terpyridine component. Moreover, with respect to the other platinum terpyridyl chloride complexes, this band possibly possesses some metal-to-ligand charge transfer character (¹MLCT). In addition, considering the large extinction coefficient of this absorption band, this band is thought to have some ¹π,π* character. The charge-transfer nature of the low-energy absorption band is supported by the pronounced negative solvatochromic effect for complex F-11 and for complex F-10, which is similar to that observed from the low-energy charge transfer absorption band in the protonated ligands. For example, the low-energy absorption band maximum for F-10 is 522 nm in hexane, which is 48 nm red-shifted compared to that in CH₃CN. Therefore the low-energy absorption band for F-10-F-12 is tentatively assigned as a mixture of ¹ILCT/¹π,π*/¹MLCT. The mixed configurationally distinct transitions in the low-energy absorption band could be further supported by the energy trend of this band, which follows F-11>F-10>F-12 and is inconsistent with that observed for the respective ligand. This could reflect a different degree of involvement of the charge transfer character in F-11 compared to that in F-10 and F-12.

Photoluminescence: All of the ligands and the platinum complexes are emissive at room temperature in solutions and at 77 K in glassy matrix. The room temperature emission spectra of 17, 18, and 19 in CH₂Cl₂ are shown in FIG. 25, and the emission data are summarized in Table 5. The emission of 17, 18 and 19 occurs at 484, 505, and 526 nm in CH₂Cl₂, respectively, with a high emission quantum yield (Φₑₘ) of 0.59, 0.70 and 0.42 for 17, 18, and 19, respectively. The emission energy decreases from 17 to 19, which is in line with the trend observed for the UV-vis absorption. The emission of the ligands is highly sensitive to the polarity of solvent. A drastic positive solvatochromic effect is observed for 17, e.g. the emission in CH₃CN (510 nm) is much red-shifted compared to that in hexane (406 nm), which is indicative of a charge-transfer emitting state. The positive solvatochromic effect suggests that the emitting excited state is more polar than the ground state, which should be the ¹ICT state. Similar phenomena were observed for 18 and 19. However, the excitation spectra monitored at the emission band maxima resemble those of the ¹π,π*/¹ICT transitions in the UV-vis absorption spectra, which implies that the emitting state could have ¹π,π* character as well. Therefore, the emission from the ligands can be regarded as a mixture of ¹ICT/¹π,π* characters. This is in line with the dual fluorescence observed in many 4-aminobenzonitrile compounds.

The assignment of the emitting state of the ligands to mixed ¹ICT/¹π,π* states can be partially supported by the emission lifetime measurement using the 355 nm laser beam as excitation source. The emission from all ligands exhibits bi-exponential decays by deconvolution of the decay curve, with a short lifetime of tens of ns being attributed to the ¹ICT emission and a longer lifetime of approximately 100 ns being assigned to the ¹π,π* emission. In addition, the emission lifetime and intensity remain the same in argon-saturated solution compared to those in air-saturated solution. These results clearly indicate that the observed emission from the ligands is fluorescence from singlet excited state that admixes ¹π,π* and ¹ICT characters.

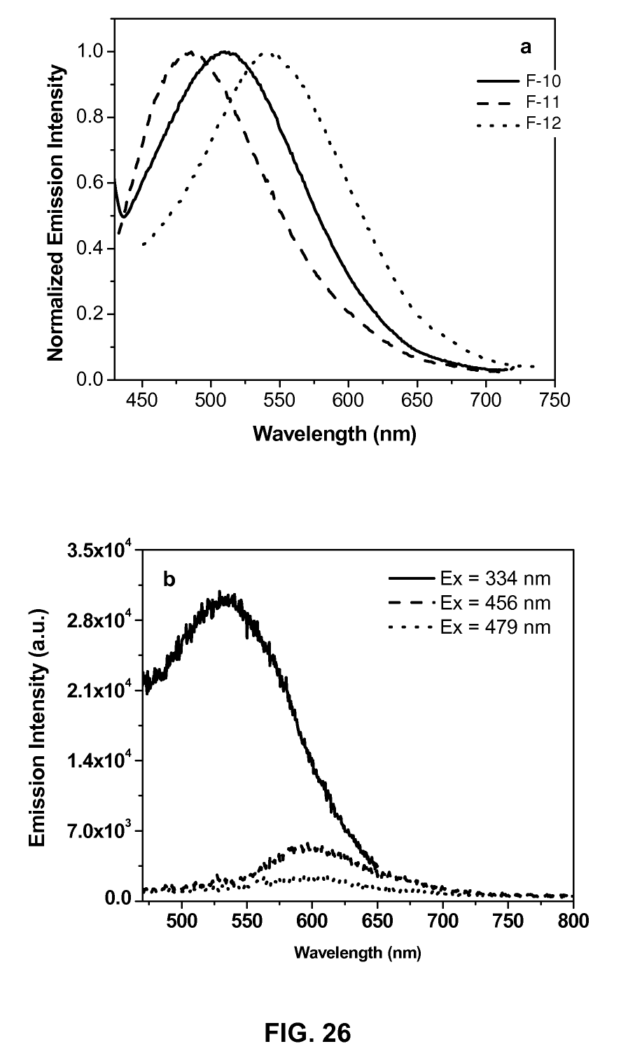

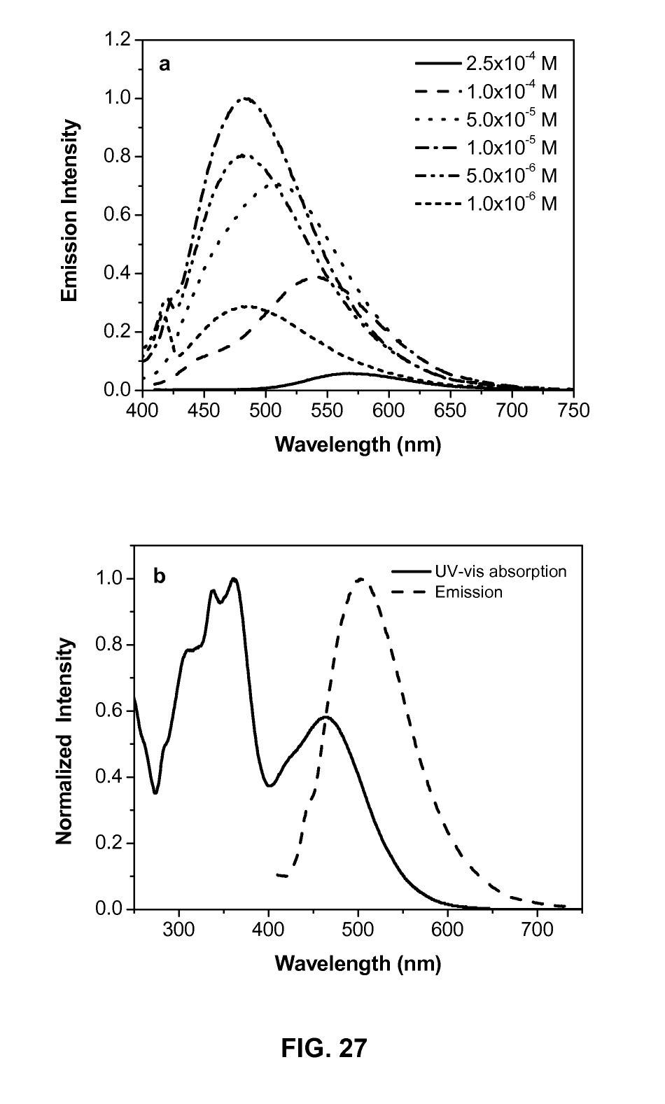

The emission spectra of platinum complexes F-10-F-12 at room temperature upon excitation at wavelengths shorter than 400 nm are shown in FIG. 26a. The emission energies for F-10-F-12 resemble those of their corresponding ligand. However, in contrast to the emission from the ligand, the emission energy of the complex decreases with increased concentration. As shown in FIG. 27 for F-11, in the concentration range of 1×10⁻⁶ and 1×10⁻⁵ mol/L, the emission intensity increases with increased concentration and the emission energy remains essentially the same. At higher concentration solutions (5×10⁻⁵−2.5×10⁻⁴ mol/L), the emission intensity decreases and the emission band maximum bathochromically shifts. In view of the significant overlap of the low-energy absorption band in the UV-vis absorption spectrum and the high-energy end of the emission band (shown in FIG. 27b), the decreased emission intensity and emission energy should be attributed to re-absorption of the emission. A similar effect is observed for F-10 and F-11. Other differences between the emission from the ligands and the complexes include the drastically reduced emission quantum yield and the much shorter lifetime of F-10, F-11, and F-12 compared to those of their corresponding ligand. On the other hand, consistent with the emission from the ligands, the emission from the complexes is also independent of oxygen. The much shorter lifetime and the oxygen-independence suggest that the emission of the complexes upon excitation below 400 nm emanates from a singlet excited state. In view of the similarity of the emission energies of the platinum complexes to those of their corresponding ligand, and of the excitation spectra of the complexes to those of the ¹π,π*/¹ICT transitions in the UV-vis absorption of the corresponding ligand, and the similar positive solvatochromic effect, the observed emission upon excitation below 400 nm is tentatively assigned to ¹π,π*/¹ILCT states.

Upon excitation at the low-energy ¹ILCT/¹π,π*/¹MLCT band, a very weak, structureless emission appears at approximately 570 nm for F-10, 590 nm for F-11, and 620 nm for F-12, as exemplified in FIG. 26b for F-11. This band becomes the dominant emission band in low-polarity solvents, such as in toluene and hexane, and a drastic negative solvatochromic effect is observed. The excitation spectra monitored at this low-energy emission band resemble the low-energy ¹ILCT/¹π,π*/¹MLCT band in their UV-vis spectra. These facts suggest that the low-energy emission band should originate from the charge transfer state(s). In view of this emission band falling into the broad envelop of the spectrum obtained at excitation below 400 nm that shows biexponential decay, the emission spectra of F-10-F-12 (FIG. 26a) obtained upon excitation below 400 nm indeed compose ¹π,π*/¹ILCT characters. However, the involvement of the ¹MLCT character cannot be excluded in view of the energy trend (F-11>F-10>F-12) observed for these complexes, which is consistent with that observed for the low-energy ¹ILCT/¹π,π*/¹MLCT absorption band. This phenomenon suggests the involvement of the ¹MLCT character in the emitting state but the degree of ¹MLCT involvement is different in F-11 compared to that in F-10 and F-12.

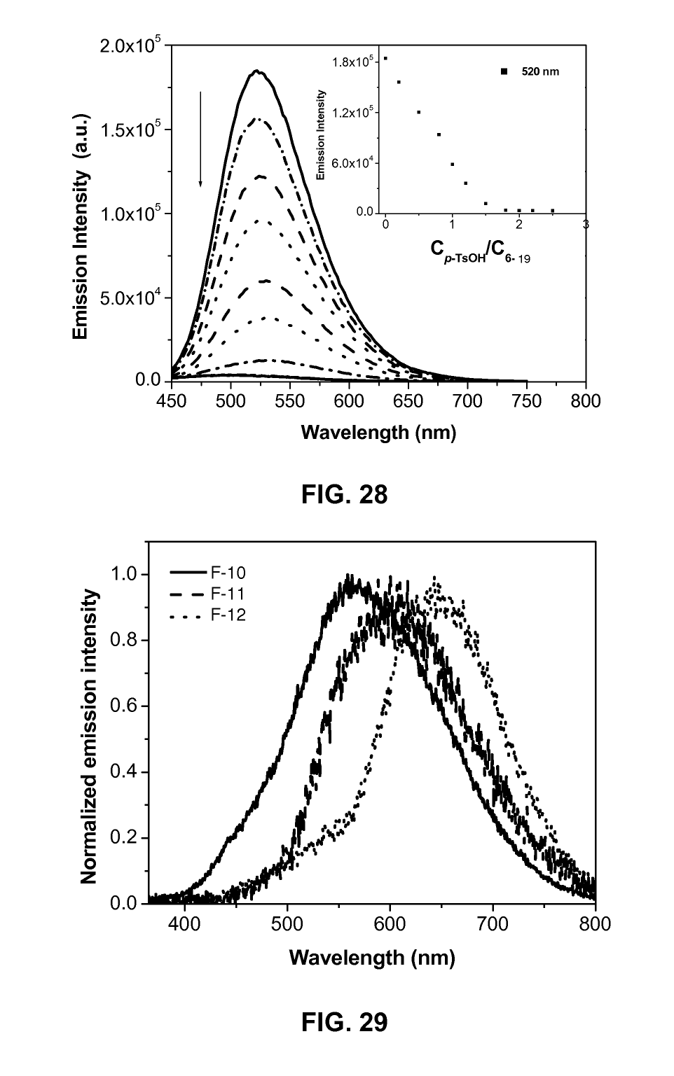

To verify the involvement of ¹ICT or ¹ILCT character into the emitting states of the ligands and the platinum complexes, the emission of the protonated ligands has been investigated. FIG. 28 shows the emission spectra of 19 with addition of p-TsOH. Upon addition of p-TsOH, the emission of 19 at ca. 502 nm decreases. This can be rationalized by the fact that protonation of the nitrogen atoms on the terpyridine increases the electron-withdrawing ability of the terpyridine component, which in turn results in enhanced ¹ICT or ¹ILCT character and quenches the emission. This result is in line with the increased ¹ICT or ¹ILCT character observed in the UV-vis absorption spectra of the ligands and the complexes, and also partially accounts for the very low emission quantum yields of the platinum complexes upon excitation at 436 nm (Table 5).

As described above, the emission of F-10-F-12 is assigned as the mixture of ¹π,π*/¹ILCT characters, similar to those of their corresponding ligands, although the emission of F-10-F-12 also possibly admixes some ¹MLCT character. However, the emission quantum yields and the emission lifetimes are much lower or shorter than those of the respective ligand. Though not wishing to be bound by a particular theory, these differences could be rationalized by the following three possible reasons. First, the coordination of platinum(II) ion with the terpyridine ligand decreases the electron-density on the terpyridine ligand, which increases the electron-withdrawing ability of the terpyridine component and enhances the charge transfer character of the complexes. Consequently, the emission from the complexes is quenched, which has been demonstrated by the ligand titration experiment. Secondly, the heavy-metal effect of the platinum increases the intersystem crossing from the singlet excited state to the triplet excited state. However, the triplet excited state is weakly-emissive due to the low-lying excited state that facilitates the decays through nonradiative relaxation to ground state; alternatively, it may decay through thermally accessible low-lying non-emissive ³d,d state. Thirdly, the decreased emission quantum yield may be also caused by the re-absorption due to the partial overlap of the low-energy absorption band and the emission spectrum.

The assignment of the emission of F-10, F-11 and F-12 at room temperature as fluorescence is further supported by the emission measurement at 77 K, which generally measures the emission from the triplet excited state. As shown in FIG. 29 for F-10, F-11, and F-12 in butyronitrile at 77 K upon excitation at 355 nm, all complexes exhibit weak and broad emission at 77 K, which is obviously red-shifted compared to the emission at room temperature upon excitation below 400 nm. The lifetimes deduced from the decay of emission are of the order of tens to hundreds of μs, indicating that the emission of all complexes at 77 K originates from the ³π,π state. The emission from the ligands 17, 18, and 19 with 10 equivalents of CH₃I in 4:1 EtOH/MeOH was also observed (CH₃I was added as the external heavy atom to promote the intersystem crossing from the singlet to the triplet excited states in order to observe the phosphorescence) and the results are summarized in Table 5. The emission lies in the similar energy levels as those of the complexes. Therefore, the emission for 17, 18, and 19 at 77 K is also attributed to the phosphorescence from the ³π,π state. However, the lifetime of the emission of 17, 18, and 19 at 77 K could not be measured due to the very weak emission.

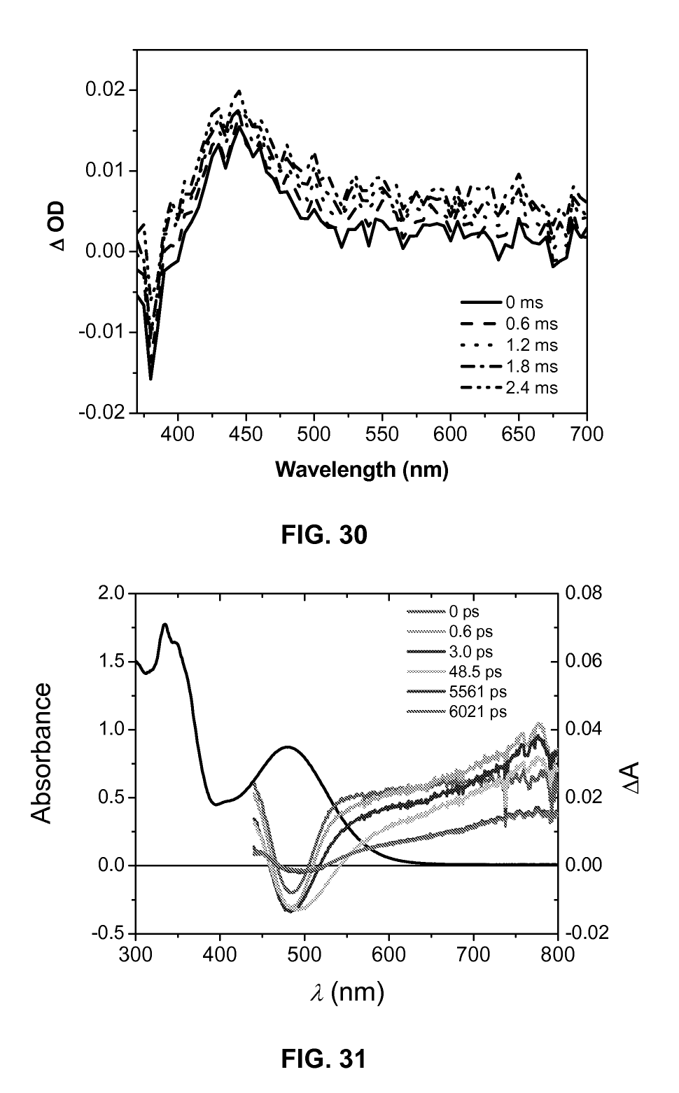

Nanosecond transient absorption: The triplet excited-state absorption of 17, 18, and 19 were studied by excitation with 4.1 ns 355 nm laser pulse. The time-resolved spectra are exemplified in FIG. 30 for 17. A positive absorption band was observed at 445, 480, and 515 nm for 17, 18 and 19, respectively. The energy of the transient absorption band maximum decreases from 17 to 19, which is consistent with the trends observed from the UV-vis absorption and the emission measurements. Because of the ultralong lifetime of the transient species, the transient absorption is thought to arise from the ³π,π* of the ligand, especially from the ³π,π* state of the fluorene component, which was reported to occur around 406 nm extending to 600 nm in the literature. In contrast, no triplet excited-state absorption was observed for F-10-F-12 at room temperature, possibly due to the short-lived triplet excited state.

Femtosecond transient absorption: F-10-F-12 exhibit fluorescence at room temperature and the transient absorption from the triplet excited state was not detected. To further investigate the singlet excited-state properties of F-10-F-12, the measurements of singlet excited-state absorption of these complexes were carried out using ultrafast femtosecond laser excitation (260 fs) at 400 nm. The transient difference absorption spectra at different decay time along with the ground-state absorption spectrum are exemplified in FIG. 31 for F-10. All complexes possess a bleaching band in the region where the ¹π,π*/¹ILCT/¹MLCT absorption band appears, and a broad, moderately strong positive absorption band from 510 nm extending to the near-IR region. The lifetimes obtained from the fitting of the decay curves are summarized in Table 6. The decay of the transient species consists of four components: a very fast decay (τ₁) due to the intramolecular vibrational relaxation (IVR) from the upper excited vibrational levels; a decay in the region of 2-4 ps (τ₂) associated with solvent reorganization around the excited molecule; a decay of tens to hundreds of ps (τ₃) and a longer decay of several ns (τ₄). The magnitude of T₃ and τ₄ coincides with the lifetime deduced from the decay of the room temperature emission from these complexes. Therefore, the excited state that gives rise to the observed transient absorption can be considered as the same excited state that emits, i.e. ¹π,π*/¹ILCT, maybe mixed with some ¹MLCT character, which is supported by the consistence of the bleaching band with the ¹π,π*/¹ILCT/¹MLCT absorption band.

TABLE 6
Femtosecond transient absorption data of F-10-F-12 in CH₃CN

| | F-10 | F-11 | F-12 |
|---|---|---|---|
| λ_{S1-Sn}/nm | 554, 780 | 549 | 590 |
| τ₁/ps | 0.5 ± 0.5 | fast | fast |
| τ₂/ps | 3.9 ± 1.7 | 2.4 ± 0.5 | 3.4 ± 1.2 |
| τ₃/ps | 62 ± 46 | 136 ± 128 | 272 ± 95 |
| τ₄/ps | 4070 ± 2262 | 2858 ± 1001 | 3945 ± 2480 |

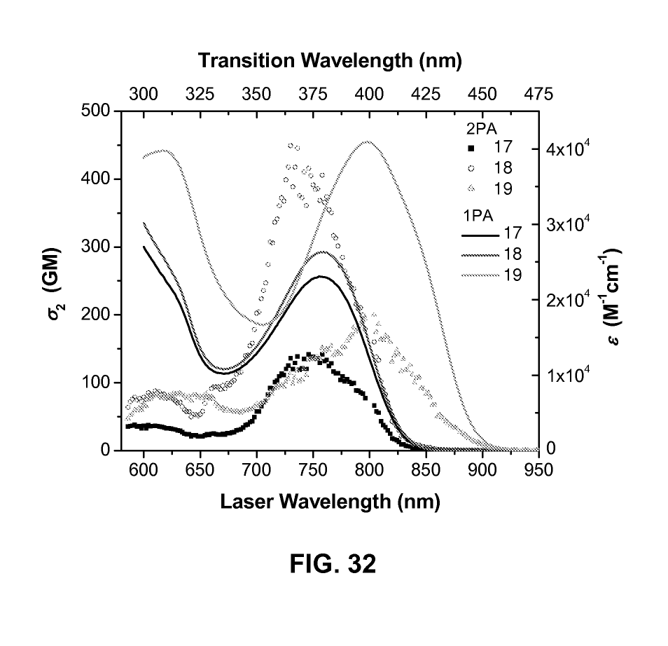

Two photon absorption: Because of the conjugated structure and the charge-transfer nature of the ligands and the complexes, it is expected that all of the ligands and the complexes exhibit at least a moderately strong two-photon absorption (2PA) (σ₂>100 GM, 1 GM=10⁻⁵⁰·cm⁴·s.photon^{-l}.molecule⁻¹) upon NIR excitation. The 2PA spectra of 17, 18, and 19 in toluene were measured by two-photon excited fluorescence method and the results are shown in FIG. 32. The 2PA band maxima of 17-19 almost coincide with their corresponding 1PA band maxima. Because of the lack of center of symmetry of these ligands and the approximate overlap with the 1PA peak, it is concluded that the lowest-energy 2PA transitions of the ligands correspond to the S₀→S₁ transitions. 17-19 exhibit the maximum σ₂ value of 142 GM, 448 GM, and 204 GM, respectively at 750 nm. The general trend of the σ₂ value follows the sequence: σ₂(18)>σ₂(19)>σ₂(17). The stronger 2PA in 18 than in 19 should be attributed to the better conjugation provided by the ethynylene bridge, which avoids the twisting of the fluorenyl component out of the conjugation in comparison to a vinylene linker.

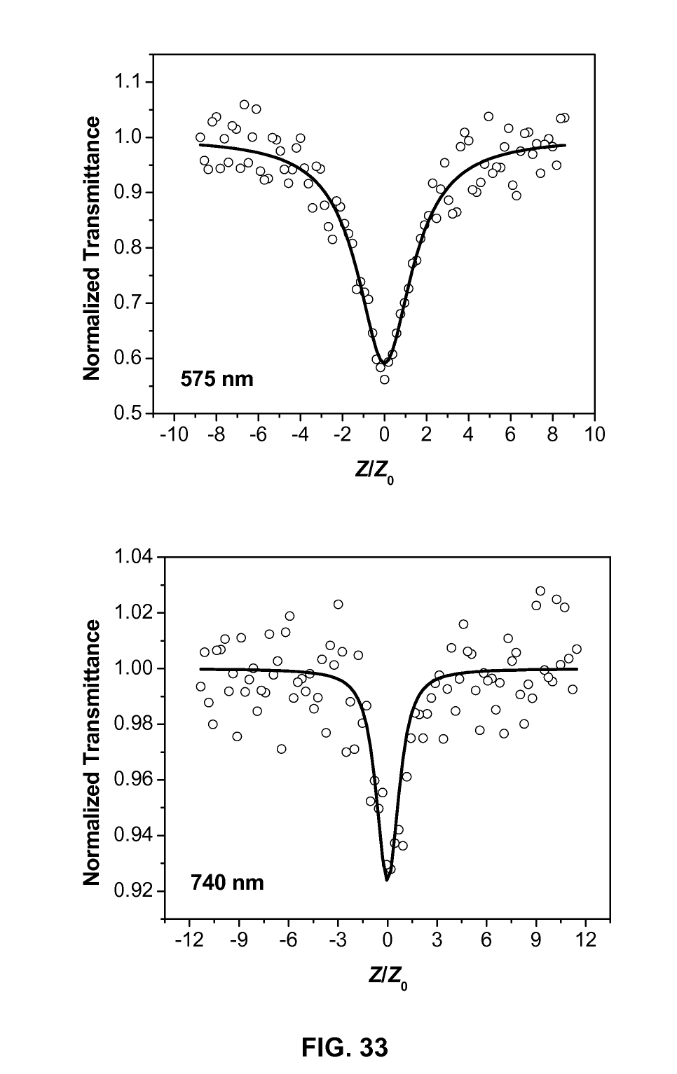

Even though the peak σ₂ values for the ligands are not particularly large, they are comparable to the values in similar structures such as BDPAS (4,4′-bis(diphenylamino)stilbene), in which σ₂=230 GM at 650 nm. After coordination with the platinum ion, the intraligand charge transfer is enhanced, which is reflected by the red-shift of the low-energy absorption band in the UV-vis absorption spectra of the complexes. Wavelength dependent open-aperture Z-scan measurements were performed using 21 ps duration NIR pulses. To properly account for possible stepwise absorption from the excited states, the experimental data was fitted using a five-band model, which includes both the excited-state absorption and the two-photon absorption. In order to disambiguate the relative contributions of two-photon and excited-state absorption at those wavelengths at which the two-photon process represents the dominant mechanism for populating the excited states, the singlet excited-state absorption cross section σ_{S}(λ) was estimated from the fs transient absorption spectrum at zero time delay and only σ₂(λ) was used as a fitting parameter. Nonlinear absorption was observed from 575 nm to 740 nm for F-10, 550 nm to 825 nm for F-11, and 575 nm to 670 nm for F-12. FIG. 33 shows the typical nonlinear absorption data and the fitting curves at two different wavelengths for F-11. For all three complexes, there is measurable ground-state absorption at the wavelengths shorter than 670 nm. The nonlinear absorption observed at these wavelengths relates most likely to the transient absorption from the excited S₁-state. Calculations of the excited-state population during one laser pulse excitation indicate that at wavelengths shorter than 670 nm, all three complexes also have significant S₂-populations (reaching a maximum of ˜20-40%). However, except for the 575-nm Z scan of F-12 (which represents something of a special case), the effects of excited-state absorption from S₁ cannot be unambiguously separated from the effects of excited-state absorption from S₂ using the data available at this time. For this reason, the σ_{S}(λ) values quoted in Table 7 should be interpreted as effective values representing a weighted average of the effects of excited-state absorption from S₁ and S₂, in which the former contribution is dominant; they were obtained by setting σ_{S}(λ) equal to σ_{S2}(λ) and fitting with a single free parameter. At wavelengths longer than 740 nm, the nonlinear absorption is attributed to the two-photon induced excited-state absorption. At each of these wavelengths the singlet excited-state absorption cross-section deduced from the fs transient absorption measurement at zero time delay was treated as a fixed parameter, and the Z-scan data were fit using the 2PA cross-section as the sole fitting parameter. As shown in Table 7, the σ₂ obtained for F-10 and F-11 are all much larger than those of their respective ligands. It is noted that F-11 exhibits a broader 2PA than F-10, and the σ₂ value is larger for F-11 than that for F-10 at the corresponding wavelength. This is consistent with the trend observed from the corresponding ligands. For complex F-12, the 2PA was too weak to be observed. The stronger 2PA in F-11 than in F-12 should also be attributed to the better conjugation provided by the ethynylene bridge, similar to that discussed earlier for the ligands. The σ₂ values obtained by the Z-scan method could be over-estimated compared to those obtained by the two-photon excited fluorescence method. Nevertheless, the trend of σ₂ values observed for these complexes should still be valid.

TABLE 7
Excited-state absorption and two-photon absorption cross-sections for
F-10-F-12 at different wavelengths.

| | λ | σ₀ | σ_{S} | | |
|---|---|---|---|---|---|
| Complex | (nm) | (10⁻¹⁸ cm²)^{[a]} | (10⁻¹⁸ cm²)^{[b]} | σ_{S}/σ₀ | σ₂ (GM) |
| F-10 | 575 | 10.1 | 20 ± 1 | 2.0 | |
| | 600 | 3.83 | 20 ± 2 | 5.2 | |
| | 630 | 0.956 | 17 ± 1 | 18 | |
| | 670 | 0.191 | 25 ± 1 | 131 | |
| | 740 | | 24.4^{[c]} | | 850 ± 50 |
| F-11 | 550 | 14.7 | 38 ± 2 | 2.6 | |
| | 575 | 6.31 | 24 ± 2 | 3.8 | |
| | 600 | 2.49 | 24 ± 2 | 9.6 | |
| | 630 | 0.765 | 26 ± 2 | 34 | |
| | 680 | 0.153 | 12 ± 1 | 78 | |
| | 740 | | 7.7^{[c]} | | 1200 ± 100 |
| | 760 | | 11.1^{[c]} | | 1000 ± 200 |
| | 800 | | 7.7^{[c]} | | 2000 ± 200 |
| | 825 | | 11.6^{[c]} | | 600 ± 100 |
| F-12 | 575 | 25.8 | 43 ± 5^{[d]} | 1.7 | |
| | 600 | 10.9 | 36 ± 2 | 3.3 | |
| | 630 | 3.63 | 20 ± 2 | 5.5 | |
| | 670 | 0.765 | 16 ± 1 | 21 | |

^{[a]}Ground-state absorption cross-section.
^{[b]}Effective singlet excited-state absorption cross-section with the assumption of σ_{S2} = σ_{S}.
^{[c]}Estimated from the fs TA data at zero time delay.
^{[d]}σ_{S2} = (12 ± 7) × 10⁻¹⁸ cm².

### Example 5

### Synthesis, Structural Characterization, Photophysics and Broadband Nonlinear Absorption of Platinum(II) Complex Bearing 6-(7-Benzothiazol-2′-yl-9,9-diethyl-9H-fluoren-2-yl)-2,2′-bipyridinyl Ligand

### Synthesis

All solvents and reagents were purchased from Aldrich or Alfa Aesar and used as is unless otherwise stated. Compounds 33, 29 and F-14 were characterized by ¹H-NMR and elemental analyses. Additional characterization by high-resolution electrospray ionization mass spectrometry (ESI-HRMS) was carried out on 29 and F-14. ¹H-NMR spectra were measured on a Varian Oxford-400 VNMR spectrometer or a Varian Oxford-500 VNMR spectrometer; and ESI-HRMS analyses were conducted on a Bruker Daltonics BioTOF III mass spectrometer. Elemental analyses were performed by NuMega Resonance Labs, Inc. in San Diego, Calif.

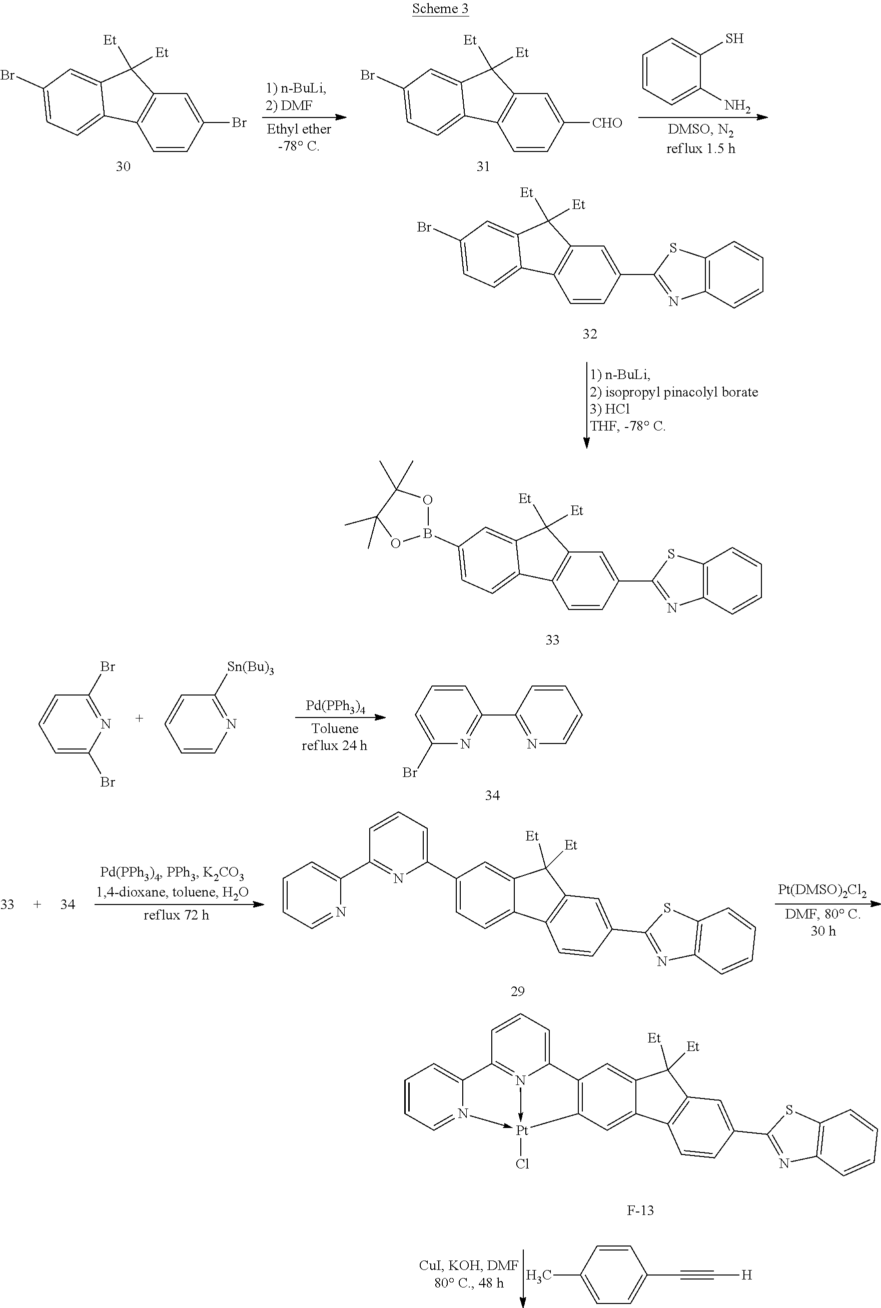

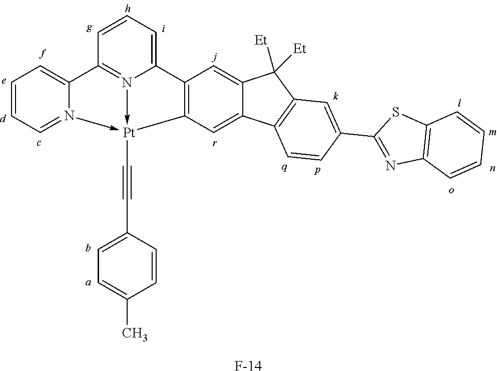

6-Bromo-2,2′-bipyridine (34), 2,7-dibromo-9,9-diethyl-9H-fluorene (30), 7-bromo-9,9-diethyl-9H-fluorene-2-carbaldehyde (31), and 2-(7-bromo-9,9-diethyl-9H-fluoren-2-yl)-benzothiazole (32) were prepared according to the procedures published in the literature.

33. Compound 32 (3.05 g, 7.00 mmol) was dissolved in degassed dry THF (40 mL) and the solution was cooled down to −78° C. in a dry ice-heptane bath. 5.1 mL n-butyl lithium in hexane (1.60 M, 8.20 mmol) was then added dropwise under argon. After stirring for 30 mins, isopropyl pinacolyl borate (1.70 mL, 1.52 g, 8.20 mmol,) was added using a syringe. The reaction mixture was stirred overnight, first at −78° C. and then slowly warmed up to room temperature. After reaction, the mixture was again cooled down to 5° C., and treated with a hydrochloric acid solution (15 mL, 6.00 M). Then THF was removed by distillation, and the aqueous phase was extracted three times with diethyl ether (3×50 mL). The organic layer was washed with brine, dried with Na₂SO₄ and the solvent was removed. The residual solid was recrystallized from toluene to give 2.66 g yellow crystal (yield: 70%). ¹H-NMR (CDCl₃): δ 8.13 (s, 1H), 8.11 (d, 1H, J=8.0 Hz), 8.04 (d, 1H, J=8.0 Hz), 7.92 (d, 1H, J=8.0 Hz), 7.80-7.86 (m, 2H), 7.74-7.80 (m, 2H), 7.51 (t, 1H, J=9.0 Hz), 7.40 (t, 1H, J=9.0 Hz), 8.04 (m, 4H), 7.99 (t, 2H, J=8.0 Hz), 7.51 (t, 2H, J=8.0 Hz), 2.10-2.22 (m, 4H), 1.40 (s, 12H), 0.28-0.33 (m, 6H). Anal. Calc. for C₃₀H₃₂BNO₂S: C, 74.8; H, 6.7; N, 2.9; Found: C, 74.8; H, 7.1; N, 3.2.

29. Compounds 33 (0.48 g, 1.00 mmol), 34 (0.35 g, 1.50 mmol), and K₂CO₃ (5.50 g, 0.04 mol) were dissolved in a mixed solvent of dioxane (40 mL), toluene (40 mL) and water (20 mL), and the solution was degassed with argon for 30 mins. Pd(PPh₃)₄ (33 mg, 0.03 mmol) and PPh₃ (16 mg, 0.06 mmol) were then added. After refluxing for 72 hrs. under argon, the aqueous phase was extracted with diethyl ether (3×50 mL). The organic layer was washed with brine, dried with Na₂SO₄ and the solvent was removed. The residual solid was purified by chromatography on silica gel (Sorbent Technologies, 60 Å, 230˜450 mesh) column. The byproduct was removed first by toluene, then the desired product was obtained by using dichloromethane or ether as the eluent. The crude product was purified by recrystallization from dichloromethane and heptane to give colorless crystal (0.35 g, yield: 70%) suitable for X-ray diffraction analysis. ¹H-NMR (CDCl₃): δ 8.72 (d, 1H, J=4.0 Hz), 8.70 (d, 1H, J=8.0 Hz), 8.44 (dd, 1H, J=8.0 Hz and 1.2 Hz), 8.24 (d, 1H, J=1.6 Hz), 8.21 (dd, 1H, J=8.0 Hz and 1.6 Hz), 8.20 (s, 1H), 8.14 (d, 1H, J=8.0 Hz), 8.05 (dd, 1H, J=8.0 Hz and 1.6 Hz), 7.79-7.90 (m, 6H), 7.47 (dt, 1H, J=8.0 Hz and 1.2 Hz), 7.36 (dt, 1H, J=8.0 Hz and 1.2 Hz), 7.26-7.32 (m, 1H), 2.20-2.28 (m, 4H), 0.46 (t, 6H, J=7.6 Hz). ESI-HRMS: m/z calcd for [C₃₄H₂₇N₃S+H]⁺: 510.1998; found, 510.1980. Anal. Calc. for C₃₄H₂₇N₃S: C, 80.12; H, 5.34; N, 8.24; Found: C, 79.85; H, 5.66; N, 8.40.

F-13. Ligand 29 (130 mg, 0.26 mmol) and Pt(DMSO)₂Cl₂ (126 mg, 0.30 mmol) were dissolved in DMF (5 mL) with a few drops of water. The solution was stirred at 80° C. for 30 hrs. under argon. The formed yellow solid was collected by filtration, washed with water, methanol and ether, and dried in vacuum. 190 mg crude product of 7 was obtained in quantitative yield. Due to the poor solubility of 7 (insoluble in ethanol, ether, hexane and toluene, slightly soluble in DMSO, DMF and CH₂Cl₂), it could not be further purified by column chromatography or recrystallization. Therefore, it was used directly in the following step for preparation of F-14 without further purification and/or characterization.

F-14. To a degassed suspension of F-13 (128 mg, 0.16 mmol) and 1-ethynyl-4-methylbenzene (23 mg, 0.20 mmol, 25 μL) in DMF (70 mL), powder KOH (14 mg, 0.25 mmol) and catalytic amount of CuI were added. The reaction mixture was heated and stirred at 80° C. for 48 hrs. under argon. After removing the solvent, the residue was washed with water and ether, dried in vacuum, and purified by column chromatography on silica gel (Sorbent Technologies, 60 Å, 230˜450 mesh) column Dichloromethane with 3% methanol (V/V) was used as the eluent. The pure product of complex 1 was obtained as orange solid (44 mg, yield: 31%). ¹H-NMR (500 MHz, CDCl₃): δ 9.24 (s, 1H), 8.44 (t, 1H, J=42 Hz), 8.15 (d, 1H, J=1.5 Hz), 8.12 (d, 1H, J=8.0 Hz), 8.03 (dd, 1H, J=8.0 Hz and 1.5 Hz), 8.01 (d, 1H, J=10 Hz), 8.88-7.96 (m, 3H), 7.82 (t, 1H, J=8.0 Hz), 7.63 (br, 2H), 7.57 (d, 2H, J=8.0 Hz), 7.52 (dt, 2H, J=8.0 Hz and 1.0 Hz), 7.41 (dt, 2H, J=8.0 Hz and 1.0 Hz), 7.36 (s, 1H), 7.18 (d, 2H, J=8.0 Hz), 2.06-2.26 (m, 4H), 0.43 (t, 6H, J=7.5 Hz). ESI-HRMS: m/z calcd for [C₄₃H₃₃N₃PtS+H]⁺: 819.2119; found, 819.2105. Anal. Calc. for C₄₃H₃₃N₃PtS.CH₂Cl₂: C, 58.47; H, 3.90; N, 4.65; Found: C, 58.33; H, 3.93; N, 4.91.

### Photophysical Measurements

The UV-vis spectra of 29 and F-14 in different solvents (spectrophotometric grade) were acquired using a UV-2501 spectrophotometer. The steady state emission spectra in different solvents were obtained on a SPEX fluorolog-3 fluorometer/phosphorometer. The emission quantum yields were measured by the relative actinometry method in degassed solutions. A degassed aqueous solution of [Ru(bpy)₃]Cl₂ (Φₑₘ=0.042, λₑₓ=436 nm) was used as the reference for complex F-14 and an aqueous solution of quinine sulfate (Φ_{f}=0.546, λₑₓ=347.5 nm) was used as the reference for ligand 29. The excited-state lifetimes, the triplet transient difference absorption spectra, and the triplet excited-state quantum yields were measured on an Edinburgh LP920 laser flash photolysis spectrometer. The third harmonic output (355 nm) of a Nd:YAG laser (Quantel Brilliant, pulsewidth ˜4.1 ns, repetition rate is set to 1 Hz) was used as the excitation source. Sample solutions were purged with Ar for 30 mins. prior to each measurement. The femtosecond transient difference absorption spectra and the singlet excited-state lifetime were measured using a femtosecond pump-probe UV-vis spectrometer (HELIOS) manufactured by Ultrafast Systems LLC. The sample solution in a 2-mm cuvette was excited at 400 nm using a 150-fs Ti:Sapphire laser (Spectra Physics Hurricane, 1 kHz repetition rate, 1 mJ/pulse at 800 nm) and the absorption was probed from 425 to 800 nm with sapphire generated white-light continuum.

The triplet excited-state molar extinction coefficients (ε_{T}) at the TA band maximum were determined by the singlet depletion method, in which the following equation was used to calculate the ε_{T}.

$${ɛ}_{T} = \frac{{ɛ}_{S} [ Δ {OD}_{T} ]}{Δ {OD}_{S}} ,$$

where ΔOD_{S} and ΔOD_{T} are the optical density changes at the minimum of the bleaching band and at the maximum of the positive band in the TA spectrum, respectively, and ε_{S} is the ground-state molar extinction coefficient at the wavelength of the bleaching band minimum. After obtaining the ε_{T} value, the triplet excited-state quantum yield was measured by relative actinometry, in which SiNc in benzene was used as the reference (ε₅₉₀=70,000 M⁻¹cm⁻¹, Φ_{T}=0.20).

### Nonlinear Optical Characterizations

The nonlinear absorption of complex F-14 was characterized by open-aperture Z-scan experiment using ns laser at 532 nm and ps laser from 450 nm to 900 nm, and by nonlinear transmission experiment at 532 nm using ns laser. The experimental data were fitted using a five-level model to extract the excited-state absorption cross sections and the two-photon absorption cross sections.

The nonlinear transmission experiment for complex F-14 was conducted in CH₂Cl₂ in a 2-mm cuvette using 4.1 ns laser pulses at 532 nm. The light source was a Quantel Brilliant ns laser with a repetition rate of 10 Hz. The experimental setup and details are the same as previously described. A 20-cm plano-convex lens was used to focus the beam to the 2-mm thick sample cuvette. The linear transmission of the solution was adjusted to 80% at 532 nm.

### Results

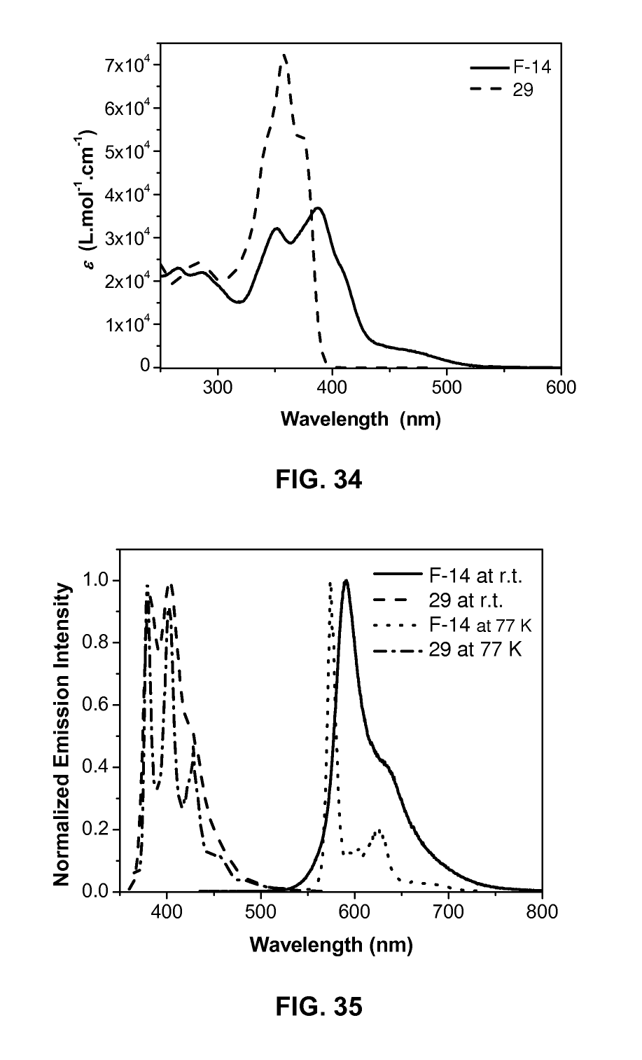

Electronic Absorption. The electronic absorption of 29 and F-14 obeys Beer-Lambert law in the concentration range used in herein (5×10⁻⁶ mol/L-5×10⁻³ mol/L). The UV-Vis absorption spectra of 29 and F-14 in CH₂Cl₂ are shown in FIG. 34. The absorption band maxima and molar extinction coefficients are presented in Table 8.

TABLE 8
Photophysical parameters of 29 and F-14.

| | | | | | λ_{T1-Tn}/nm |
|---|---|---|---|---|---|
| | | | | | (ε_{T1-Tn}/ |
| | λ_{abs}/nm | λₑₘ/nm | λₑₘ/nm (τ/μs)^{c} | λ_{S1-Sn}/nm | L · mol⁻¹ · cm⁻¹; |
| | (log ε/L · mol⁻¹ · cm⁻¹)^{a} | (Φₑₘ; τ₀)^{b} | 77 K | (τ_{S}/ps)^{d} | τ_{TA}/μs; Φ_{T})^{e} |
| 29 | 282 (4.39), 358.5 | 380, 410 | 379, 402 | 646 | 585 (83290; |
| | (4.86), 373 (4.73) | (0.73; 752 ps) | (0.011 (16%), | (796 ± 96) | 32.8; 0.36)^{f} |
| | | | 0.090 (84%)), | | |
| | | | 427, 453 | | |
| F- | 265 (4.36), 286 (4.34), | 591 (0.067; | 575 (19.4), | 633 | 633 (44650; |
| 14 | 352 (4.51), 387.5 (4.57), | 1430 ns), 634 | 625 (21.1) | (24.6 ± 14.8) | 14.0; 0.28)^{g} |
| | 458 (3.63) | (—; 1380 ns) | | | |

^{a}UV-vis absorption band maxima and molar extinction coefficients in CH₂Cl₂.
^{b}Emission band maximum, quantum yield, and lifetime in CH₂Cl₂ at a concentration of 5 × 10⁻⁶ mol/L.
^{c}Emission band maxima and lifetime in butyronitrile matrix at 77 K at a concentration of 5 × 10⁻⁶ mol/L.
^{d}fs TA band maximum and singlet excited-state lifetime in CH₂Cl₂.
^{e}ns TA band maximum, triplet extinction coefficient, triplet excited-state lifetime and quantum yield.
^{f}Measured in butyronitrile.
^{g}Measured in CH₂Cl₂•SiNc in C₆H₆ was used as the reference. (ε₅₉₀ = 70,000 L · mol⁻¹ · cm⁻¹, Φ_{T} = 0.20)

The absorption of 29 is dominated by structured bands in the UV region, which emanate from the ¹π,π* transitions. The polarity of solvent exhibits minor effect on the UV-Vis spectrum of 29, which is consistent with the ¹π,π* assignment. For complex F-14, the dominant absorption also appears in the UV region, however, the bands are red-shifted compared to those in 29, indicating the delocalization of the ligand centered molecular orbitals through interactions with the platinum dπ orbitals. Considering the similarity in energy of these bands for F-14 and 29 and the large molar extinction coefficients of these bands in F-14, these bands can be assigned to ¹π,π* transitions within the 6-(7-benzothiazol-2′-yl-9,9-diethyl-9H-fluoren-2-yl)-2,2′-bipyridine ligand (predominantly within the benzothiazolylfluorene component) as well. In addition, a broad, structureless tail between 430 nm and 530 nm is observed in complex F-14, but not in ligand 29. With reference to other Pt(II) ĈN̂N and terpyridyl acetylide complexes, this tail could be attributed to the ¹MLCT/¹LLCT (metal-to-ligand/ligand-to-ligand charge transfer) transitions. The assignment of this low-energy absorption band is bolstered by DFT calculations, in which the HOMO is dominated by the tolylacetylide ligand and the Pt components, and the LUMO has major contribution from the bipyridine component. Another piece of evidence that supports the charge transfer nature of the low-energy absorption band is the negative solvatochromic effect. This band shifts to a longer wavelength in less polar solvents, such as toluene and hexane, in comparison to those in more polar solvents (CH₃CN, CH₂Cl₂ and DMSO). This is indicative of the charge transfer character of the ground state.

Emission. 29 and F-14 are both emissive in solutions at room temperature and in glassy matrix at 77 K. As shown in FIG. 35, upon excitation of 29 at 356 nm in CH₂Cl₂ solution, it exhibits structured emission at 380 and 410 nm, which decays with a lifetime of 752 ps. The quantum yield of emission is 73%. Considering the mirror image of the emission spectrum to its UV-Vis absorption spectrum and the lifetime, the observed emission from 29 is attributed to fluorescence from the ¹π,π* state. The assignment of the ¹π,π* state as the emitting state of 29 is supported by the minor solvent effect. In different solvents with a broad range of polarity, the emission energies and quantum yields (except in hexane) are quite similar. The only difference is the relative intensity of the vibronic peaks.

For complex F-14, upon excitation at 388 nm, a broad, somewhat structured emission appears at 591 nm with a shoulder at ca. 634 nm. The vibronic spacing between the peak and the shoulder is approximately 1150 cm⁻¹, corresponding to the ring breathing mode of the aromatic rings in the ligands. The lifetime of the emission is approximately 1.43 μs. In view of the large Stokes shift of the emission and the long lifetime, the emission from F-14 at room temperature should originate from a triplet excited state. The vibronic structure in the emission spectrum suggests that the ³π,π state should be involved in the emission, possibly mixed with some ³MLCT character, which is partially supported by the negative solvatochromic effect of the emission. In less polar solvents, such as in toluene and hexane, the emission spectra are red-shifted and become less structured in contrast to those in more polar solvents, such as ethanol, acetonitrile, CH₂Cl₂ and acetone. This probably is indicative of different degrees of mixing ³MLCT character into ³π,π state in solvents with different polarities. In polar solvents, the less polar ³MLCT state (in comparison to the more polar MLCT ground state) is less stabilized than the ground state, which would cause the blue-shift of the ³MLCT emission. In contrast, the influence of the solvent polarity on the ³π,π excited state is not as significant as that on the ³MLCT state. This would allow for the ³MLCT state and the ³π,π state to be energetically more close to each other in polar solvents, resulting in more configurational mixing of ³π,π and ³MLCT characters in the emission in polar solvents. On the other hand, in less polar solvents, the ³MLCT state would be more stabilized and its energy level would be lowered, while the energy of the ³π,π state is less affected. Consequently, the energy gap between these two states becomes larger and the contribution from the ³π,π state is reduced. This is reflected by the less structured emission spectra and shorter lifetime in less polar solvents. When the concentration of the CH₂Cl₂ solution increases from 1×10⁻⁶ mol/L to 1×10⁻⁴ mol/L, the intensity of the emission keeps increasing and the lifetime remains the same, suggesting that no self-quenching occurs in the concentration range used for our study.

The emission spectra of 29 and F-14 in butyronitrile matrix at 77 K are given in FIG. 35. For 1-L, the emission spectrum at 77 K exhibits clear vibronic structures but remains the same energy as that at room temperature, and the lifetime was shorter than 100 ns. Therefore, it is still fluorescence from the ¹π,π state. The emission spectrum of F-14 at 77 K becomes narrower and blue-shifted compared to that at room temperature, which is due to the rigidochromic effect. The vibronic spacing is approximately 1390 cm⁻¹, which is also consistent with the stretching vibration of the aromatic ligand. Considering the small thermally induced Stokes shift (Δ,Eₛ˜470 cm⁻¹), the similar shape and vibronic spacing of the spectra at 77 K and at room temperature for F-14, the emission of F-14 at 77 K is tentatively assigned as the ³π,π* state, possibly mixed with some ³MLCT character.

Transient Absorption Spectroscopy. Transient difference absorption spectroscopy measures the difference between the excited-state absorption and the ground-state absorption. Thus, it can provide information on the spectral region where the excited-state absorption is stronger than that of the ground-state and predict the wavelength region where reverse saturable absorption could occur. From the decay of the transient absorption, the lifetimes of the excited state giving rise to the excited-state absorption are obtained. This is especially important for measuring the singlet excited-state lifetime of the Pt(II) complexes that cannot be obtained from the decay of fluorescence because of the lack of fluorescence at room temperature in many cases. By estimating the triplet excited-state molar extinction coefficient at the triplet excited-state absorption band maximum using the singlet depletion method, and using the relative actinometry, with SiNc in benzene as the reference, the triplet excited-state quantum yield can be obtained. Both the singlet and triplet transient difference absorption spectra of 29 and F-14 were measured using fs and ns pump-probe UV-Vis spectrometers, respectively.

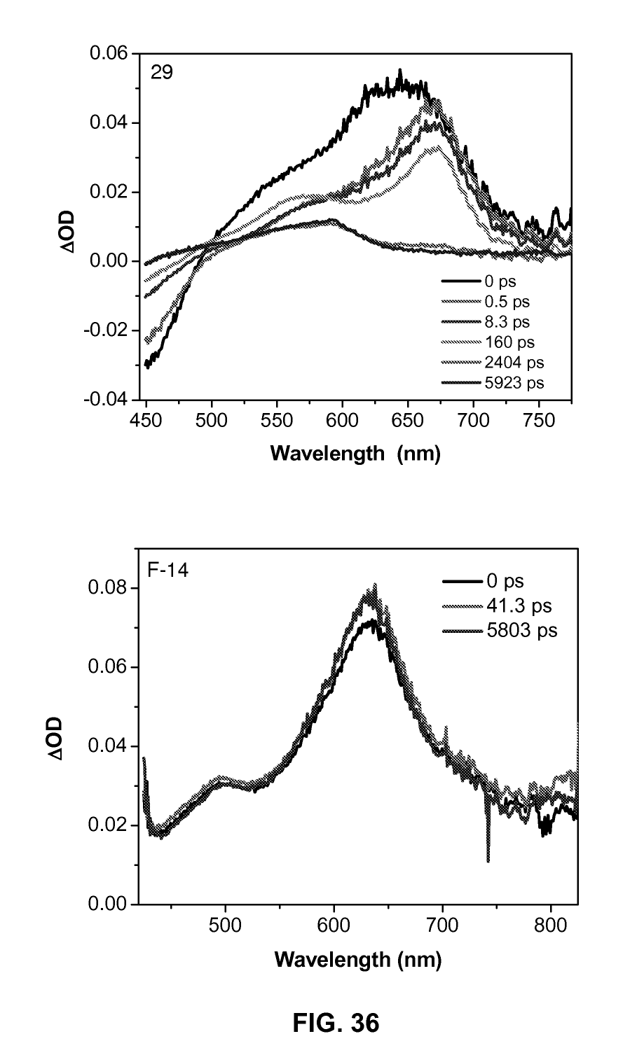

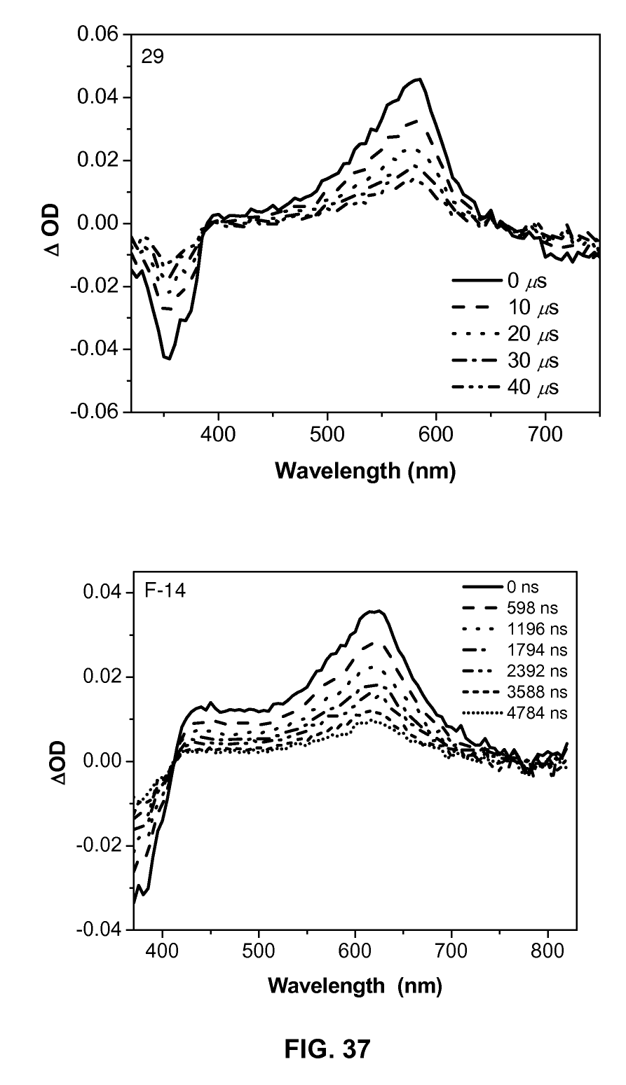

FIG. 36 shows the time-resolved singlet transient difference absorption spectra of 29 and F-14 in CH₂Cl₂. For 29, immediately after the excitation at 400 nm using ultrafast femtosecond laser pulses (150 fs), a broad, slightly structured absorption band appears at ca. 650 nm, which decays rapidly and red-shifts to ca. 675 nm. At longer decay time, a new broad band occurs at ca. 580 nm, accompanied by an isosbestic point at 480 nm. This reflects the intersystem crossing from the singlet excited state to the triplet excited state. The spectrum at longer delay times is consistent with that measured by ns laser flash photolysis (shown in FIG. 37). The singlet lifetime measured from the decay of fs TA is quite similar to that obtained from fluorescence decay (cf. Table 8). Therefore, the observed singlet TA is attributed to the ¹π,π* state, while the TA at long decay time should arise from the ³π,π* state. For complex F-14, the fs TA spectra changes very little in the whole spectrometer decay window (6 ns) although it exhibits a very small change right after the excitation, which correlates to a lifetime of approximately 25 ps. This is likely due to decay of the singlet excited state including intersystem crossing to the triplet excited state. At longer decay time, the spectrum is essentially the same as that measured by ns flash photolysis and is attributed to the triplet excited-state absorption. Since there is not much difference in the spectral properties of the singlet and triplet excited states it is likely that the geometry of the molecule does not change much upon intersystem crossing. In addition, the TA spectrum of F-14 is quite similar to that of 29, therefore, the singlet excited state that contributes to the observed TA could be assigned as the ¹π,π* and the triplet state as the ³π,π* as well. However, the singlet lifetime of the Pt complex F-14 is much shorter compared to that of the ligand 29. This is attributed to the increased intersystem crossing via spin-orbital coupling through Pt, which makes the decay of the singlet excited state more rapid.

The time-resolved triplet transient difference absorption spectra of 29 in butyronitrile and F-14 in CH₃CN are presented in FIG. 37. The spectral features for 29 and F-14 are quite similar, with a positive absorption band appearing in the visible spectral region and a bleaching band below 400 nm for 29 and below 415 nm for F-14. However, the spectrum of F-14 is red-shifted compared to that of 29, which is reflected by the absorption band maximum, i.e. 620 nm for F-14 and 585 nm for 29. The red-shifted TA spectrum of F-14 compared to that of 29 suggests the delocalization of the ligand centered molecular orbitals through interactions with the platinum dπ orbitals, which is similar to that observed in the UV-Vis absorption spectrum for the singlet excited state. The extinction coefficients at the band maximum, the lifetimes deduced from the decay of the transient absorption, and the triplet excited-state quantum yield determined from relative actinometry for 29 in butyronitrile and F-14 in CH₂Cl₂ are listed in Table 8 (the data for F-14 in CH₂Cl₂ rather than those in CH₃CN are listed because it was necessary to use the triplet excited-state parameters in CH₂Cl₂ to fit the Z-scan data measured in CH₂Cl₂ solution). The triplet extinction coefficients for both 29 and F-14 are quite large, accompanied by long triplet lifetimes. The long lifetime of 32.8 μs for 29 implies that the transient absorption likely arises from the ³π,π* state. For complex F-14, although the lifetime (14.0 μs in CH₂Cl₂) of the excited state that gives rise to the transient absorption is shorter than that of the ligand, which is probably due to the increased decay from T₁ to S₀ by spin-orbital coupling through Pt, or possibly that the reduced energy level of the ³π,π* excited state (evident by the red-shifted TA band) in F-14 leads to reduced lifetimes according to the energy gap law, it is still much longer than that deduced from the decay of emission. This indicates that the transient species would likely arise from the ³π,π* state, which is supported by the similar features of the TA spectra of F-14 and 29. The assignment of the absorbing excited state in CH₂Cl₂ solution to ³π,π* rather than the ³MLCT state or a mixed state is also partially based on the fact that the lifetimes deduced from the decay of TA in toluene (13.5 μs) is similar to that in CH₂Cl₂. This phenomenon is distinct from the solvent-dependency lifetime deduced from the decay of emission, in which the emitting state has different composition of ³π,π*/³MLCT character with varied solvent polarity. Therefore, the excited state giving rise to the transient absorption in CH₂Cl₂ and toluene solutions is the ligand-centered ³π,π* state that is not affected pronouncedly by the polarity of solvents. However, in more polar, coordinating solvent like CH₃CN, the lifetime (2.0 μs) deduced from the decay of the transient absorption is much shorter than those in CH₂Cl₂ and toluene solutions, suggesting that the ³MLCT state plays a role in transient absorption in CH₃CN solution.

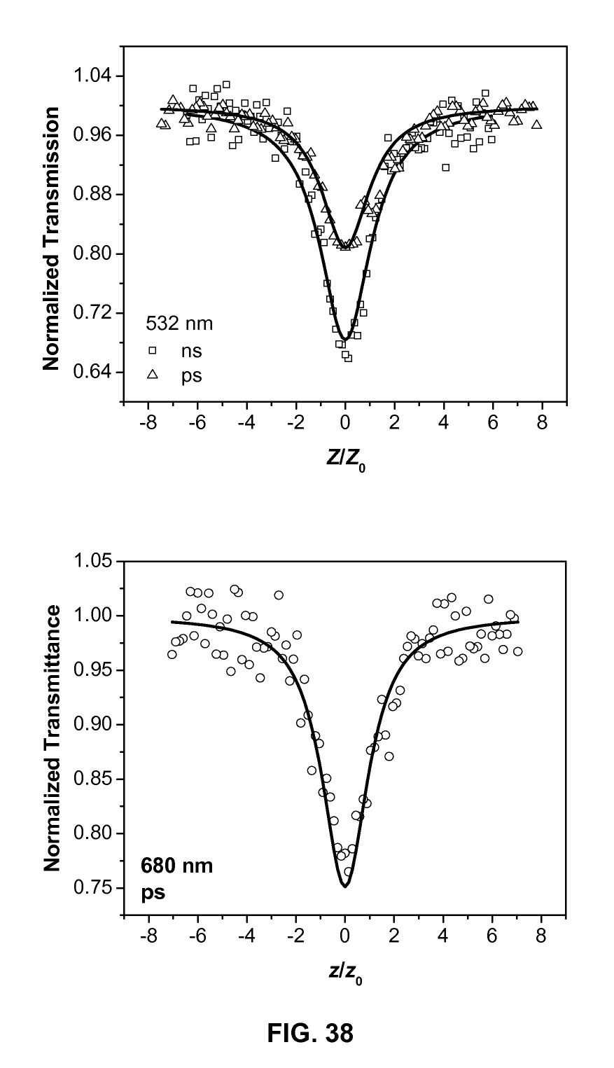

Z-scan Study and Nonlinear Absorption Cross Sections. Z-scan is a simple nonlinear optical characterization technique that is used to separately measure the contributions of nonlinear absorption and nonlinear refraction to the observed optical nonlinearity of a material. To obtain both the singlet and triplet excited-state absorption cross sections and to separate the contribution of two-photon absorption from excited-state absorption in the near-IR region, open-aperture Z scans were performed in CH₂Cl₂ solution at 532 nm using both ns and ps laser pulses and at a variety of visible and near-IR wavelengths using ps pulses. The experimental data were then fitted using the five-level model with the input parameters (σ₀, τₛ, τ_{T}, Φ_{T}) obtained from the photophysical studies described above. In this way, it was possible to obtain values for the triplet and singlet excited-state absorption cross sections at various wavelengths. Representative Z-scan data and fitting curves are provided in FIG. 38. Table 9 lists the resulting values of the excited-state absorption cross sections at wavelengths in the visible region, together with the ratio of the excited-state absorption cross section relative to that of the ground-state; also shown in Table 9 are the values of the two-photon absorption cross sections in the near-IR region.

TABLE 9
Excited-state absorption cross sections and two-photon absorption cross
sections of F-14 at selected wavelengths in CH₂Cl₂ solution.

| | | σ_{S}^{c} | | | | |
|---|---|---|---|---|---|---|
| λ/nm | σ₀ | 10⁻¹⁸ cm² | σ_{T} | σ_{S}/σ₀ | σ_{T}/σ₀ | σ₂/GM |
| 430 | 29.6 | 40 | 92^{†} | 1.4 | 3.1 | — |
| 475 | 14.3 | 30 | 101^{†} | 2.1 | 7.1 | — |
| 500 | 6.75 | 48 | 107^{†} | 7.1 | 15.9 | — |
| 532 | 1.48 | 40 ± 5 | 103 ± 10 | 27 | 69.6 | — |
| 550 | 1.25 | 40 | 115^{†} | 32 | 92 | — |
| 575 | 0.344 | 40 | 154^{†} | 116 | 448 | — |
| 600 | 0.0956 | 45 | 195^{†} | 471 | 2.04 × 10³ | — |
| 630 | 0.0318 | 200 | 253^{†} | 6.29 × 10³ | 7.96 × 10³ | — |
| 680 | 0.01 | 180 | 148^{†} | 1.80 × 10⁴ | 1.48 × 10⁴ | — |
| 740 | ~0 | 40* | 86^{†} | — | — | 600 |
| 800 | ~0 | 27* | 89^{†} | — | — | 650 |
| 850 | ~0 | — | — | — | — | 1200^{‡} |
| 875 | ~0 | — | — | — | — | 220^{£} |
| 910 | ~0 | — | — | — | — | 200^{£} |

*The starred values of σ_{S}(λ) are determined from the value σ_{S}(532 nm) = 4.0 × 10⁻¹⁷ cm² and the femtosecond transient difference absorption (fs TA) spectrum at zero time delay. Because the fs TA will include contributions from both S₁ and S₂ these values should be considered effective cross sections for the singlet excited states.
^{†}σ_{T}(532 nm) = 1.03 × 10⁻¹⁶ cm² was determined from combined fitting of nanosecond and picosecond Z-scan data. For other wavelengths, σ_{T}(λ) is determined from the value of σ_{T}(532 nm) and the fs TA spectrum at 5.8-ns time delay.
^{‡}Effective two-photon absorption cross section for the Z scan of lowest energy (0.5 J/cm²). Z scans at a progression of higher energies (0.7 and 1.1 J/cm²) yield effective σ₂ values of 1900 GM and 2000 GM, respectively, clear evidence for two-photon-initiated excited-state absorption.
^{£}Effective two-photon absorption cross section for the Z scan of 0.3 J/cm² fluence on axis.

The data in Table 9 show that the excited-state absorption cross sections at all of the wavelengths studied are in the range of 10⁻¹⁷ cm² to 10⁻¹⁶ cm², which is comparable to or even larger than those reported in the literature for other reverse saturable absorbers. The combination of strong excited-state absorption and weak ground-state absorption in the visible and near-IR leads to large ratios of the excited-state to ground-state absorption cross sections. The ratio becomes extremely large at longer wavelengths, which places it among the largest ratios reported to date for reverse saturable absorbers. The ratios of F-14 are larger than most of the reported reverse saturable absorbers. Although the ratios of F-14 are smaller than those for platinum 2,2′-bipyridine complex bearing 2-(benzothiazol-2′-yl)-9,9-diethyl-7-ethynylfluorene ligands at multiple wavelengths, the reverse saturable absorption spectral region for F-14 (430-680 nm) is broader than that (450-600 nm) for the platinum 2,2′-bipyridine complex. This feature along with the large absorption cross-section ratios, as well as the long triplet excited-state lifetime makes complex F-14 a very promising broadband reverse saturable absorber.

In the near-IR region, where the ground-state absorption is extremely weak, the excited state is populated by two-photon absorption. The observed nonlinear absorption is thus two-photon initiated excited-state absorption. The singlet and triplet excited-state absorption cross sections at 740 nm and 800 nm were estimated from the values at 532 nm and the femtosecond transient difference absorption spectra at zero and 5.8 ns time delays, respectively, and these estimated values were used as parameters in the model, which allowed the two-photon absorption cross sections at these wavelengths to be obtained by fitting the Z-scan data. At wavelengths of 850 nm and above, femtosecond transient difference absorption data were not available, so this method could not be used to provide an estimate of the excited-state absorption cross sections. As the relative contributions of two-photon and excited-state absorption cannot be unambiguously deconvolved for wavelengths of 850 nm, 875 nm, and 910 nm, the two-photon absorption cross sections given in Table 9 for these wavelengths represent effective values. The two-photon absorption cross sections of F-14 are among the largest values reported for platinum complexes.

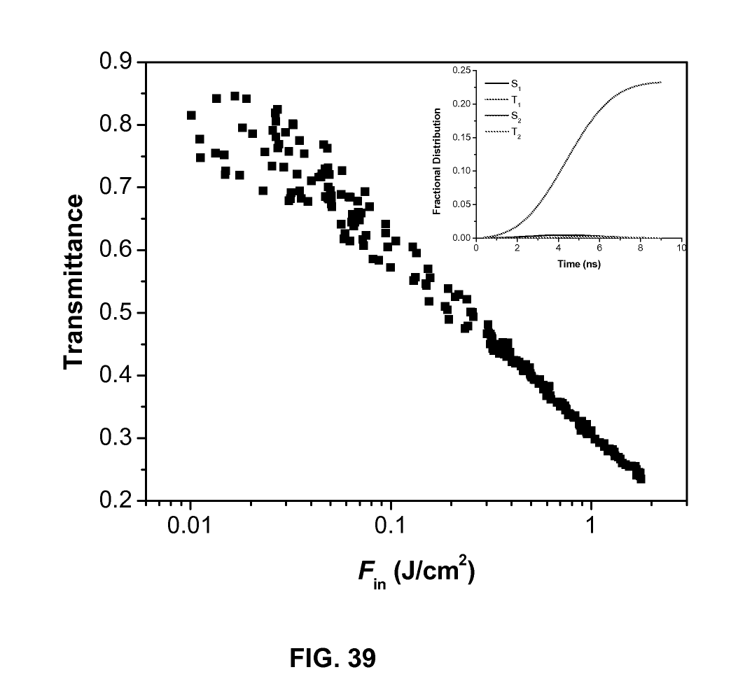

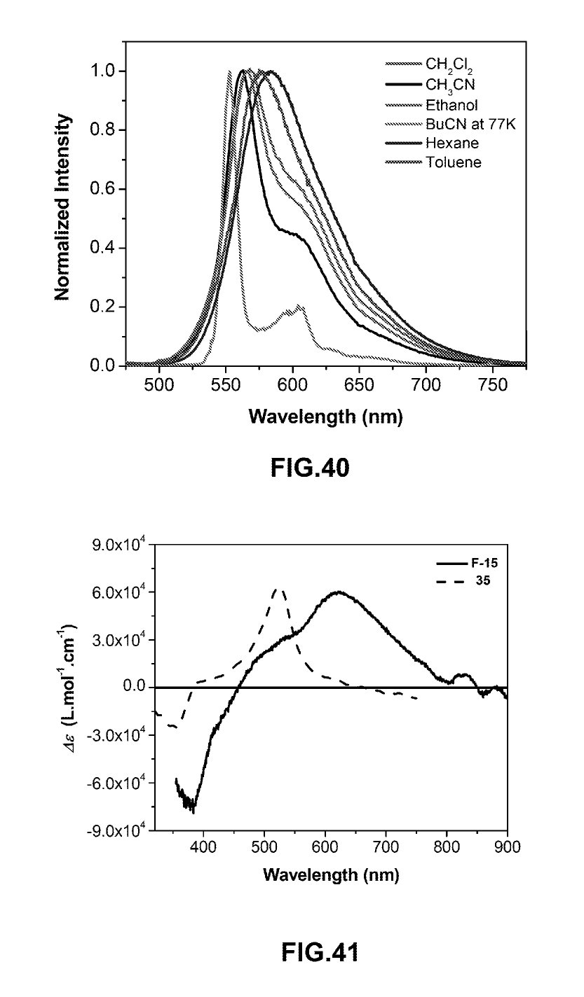

Reverse Saturable Absorption. Both the fs and ns transient absorption measurements suggest that complex F-14 exhibits stronger excited-state absorption than ground-state absorption in the visible to the near-IR region. Therefore, reverse saturable absorption in this spectral region should occur. To demonstrate this, a nonlinear transmission experiment was carried out at 532 nm using 4.1 ns laser pulses. The result is shown in FIG. 39. The transmission of the solution decreases drastically from 80% at low incident fluence to 24% at 1.8 J/cm². From the fractional populations of the affected excited states (the inset in FIG. 39) it is clear that the triplet excited state is the dominant contributor to the observed decrease in transmission. Since the triplet excited-state absorption cross section greatly exceeds that of the ground-state (σ_{T}/σ₀=115, see Table 9), strong reverse saturable absorption occurs.

### Example 6

### Broadband Nonlinear Absorbing Platinum 2,2′-Bipyridine Complex Bearing 7-(Benzothiazol-2′-yl)-9,9-diethyl-2-ethynylfluorene Ligands

All of the reagents were purchased from Aldrich Chemical Company or Alfa Aesar and used as is. The ligands 4,4′-di(tert-butyl)-2,2′-bipyridine (tBu₂ bpy) and 2-(benzothiazol-2′-yl)-9,9-diethyl-7-ethynylfluorene (35), and the precursor (tBu₂ bpy)PtCl₂ were prepared according to the literature procedures. All of the solvents purchased from VWR International are HPLC grade and used without further purification unless otherwise stated. The precursors and complex F-15 were characterized by ¹H NMR, electrospray ionization mass spectrometry (ESI-MS) and elemental analyses. ¹H NMR spectra were obtained on a Varian Oxford-400 VNMR spectrometer or a Varian Oxford-500 VNMR spectrometer. ESI-MS analyses were performed with a Bruker BioTOF III mass spectrometer. Elemental analyses were carried out by NuMega Resonance Laboratories, Inc. in San Diego, Calif.

### Synthesis of Complex F-15

The ligand 35 (200 mg, 0.53 mmol) and (tBu₂bpy) PtCl₂ (134 mg, 0.25 mmol) were dissolved in degassed dry CH₂Cl₂ (50 mL) and diisopropyl amine (5 mL). The catalyst CuI (˜5-10 mg) was then added. The reaction mixture was refluxed under argon for 24 hrs. After cooling to room temperature, the reaction solution was washed with brine, dried with Na₂SO₄ and the solvent was removed. The residual solid was purified by column chromatography on silica gel using CH₂Cl₂ as the eluent. The product was further purified by recrystallization from dichloromethane and hexane to yield yellow needle crystal 0.21 g (yield: 64%). ¹H NMR (400 MHz, CDCl₃): δ 9.75 (d, 2H, J=6.0 Hz), 8.08 (s, 2H), 8.06 (d, 2H, J=8.4 Hz), 8.00 (dd, 2H, J=8.0 Hz, 1.6 Hz), 7.96 (s, 2H), 7.88 (d, 2H, J=8.0 Hz), 7.72 (d, 2H, J=8.0 Hz), 7.64 (d, 2H, J=8.0 Hz), 7.55-7.63 (m, 6H), 7.46 (dt, 2H, J=8.0 Hz, 1.2 Hz), 7.34 (dt, 2H, J=8.0 Hz, 1.2 Hz), 7.26-7.32 (m, 1H), 2.03-2.15 (m, 4H), 1.43 (s, 18H), 0.36 (t, 6H, J=7.6 Hz). ESI-HRMS: m/z calcd for [C₇₀H₆₄N₄PtS₂+H+Na]⁺: 1243.4130; found, 1243.4123. Anal. Calc. for. C₇₀H₆₄N₄PtS₂.C₆H₁₄, C, 69.86; H, 6.02; N, 4.29; found: C, 69.67; H, 6.31; N, 4.57.

### Photophysical Measurements

The UV-vis absorption spectra were acquired on an Agilent 8453 spectrophotometer in different HPLC grade solvents. The steady-state emission spectra were recorded on a SPEX fluorolog-3 fluorometer/phosphorometer in different solvents. The emission quantum yields were determined by the relative actinometry method in degassed solutions, in which a degassed aqueous solution of [Ru(bpy)₃]Cl₂ (Φₑₘ=0.042, λₑₓ=436 nm) was used as the reference. The femtosecond transient absorption measurements were performed using a femtosecond pump-probe UV-vis spectrometer (HELIOS) manufactured by Ultrafast Systems LLC. The sample solution in a 2-mm cuvette was excited at 400 nm using a 150-fs Ti:Sapphire laser (Spectra Physics Hurricane, 1 kHz repetition rate, 1 mJ/pulse at 800 nm) and the absorption was probed from 425 to 800 nm with sapphire generated white-light continuum. The emission lifetime and the triplet transient difference absorption (TA) spectrum and the decay time were measured in degassed solutions on an Edinburgh LP920 laser flash photolysis spectrometer. The third harmonic output (355 nm) of a Nd:YAG laser (Quantel Brilliant, pulsewidth ˜4.1 ns, repetition rate was set at 1 Hz) was used as the excitation source. Each sample was purged with Ar for 30 minutes before each measurement.

The triplet excited-state absorption coefficient (ε_{T}) at the TA band maximum was determined by the singlet depletion method. The following equation was used to calculate the ε_{T}.

$${ɛ}_{T} = \frac{{ɛ}_{S} [ Δ {OD}_{T} ]}{Δ {OD}_{S}}$$

where ΔOD_{S} and ΔOD_{T} are the optical density changes at the minimum of the bleaching band and the maximum of the positive band in the TA spectrum, and ε_{S} is the ground-state molar extinction coefficient at the wavelength of the bleaching band minimum. After obtaining the ε_{T} value, the triplet excited-state quantum yield could be obtained by the relative actinometry, in which SiNc in benzene was used as the reference (ε₅₉₀=53,400 M⁻¹cm⁻¹, Φ_{T}=0.20).

### Z-Scan Measurements and Fittings

The open aperture Z-scan measurements were carried out using ns laser at 532 nm and ps laser from 450 nm to 900 nm. The experimental setup and experimental details were similar to those reported previously by our group. The experimental data were fitted using a five-level model.

### Nonlinear Transmission Measurement

The nonlinear transmission experiment was carried out using 4.1 ns laser pulses at 532 nm. A Quantel Brilliant ns laser with a repetition rate of 10 Hz was used as the light source. The focal length of the plano-convex lens used to focus the beam to the 2-mm thick sample cuvette was 20 cm.

### Results

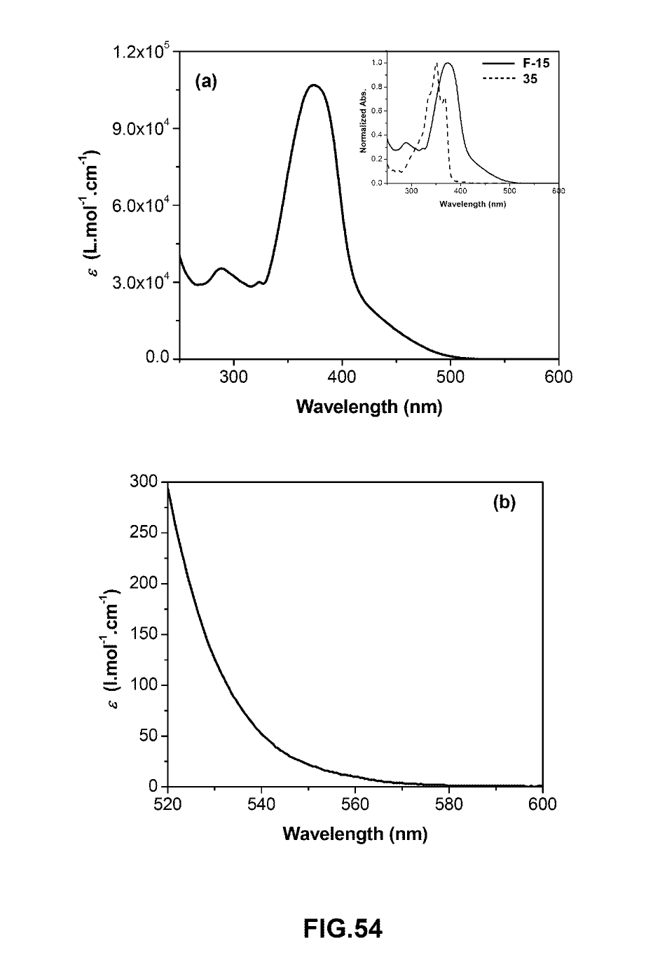

Electronic absorption. The UV-vis absorption of F-15 obeys Lambert-Beer's law in the concentration range used in our study (2×10⁻⁶ mol/L-1×10⁻⁴ mol/L), indicating that no ground-state aggregation occurs in this concentration range. FIG. 54 displays the UV-vis absorption spectrum of F-15 in CH₂Cl₂ solution. The spectrum of 35 is presented in the inset for comparison. The molar extinction coefficients are provided in Table 10. The absorption spectrum of F-15 is dominated by a broad, structureless band at ca. 374 nm, which is red-shifted approximately 20 nm compared to the absorption band of 35. The similarity in the energy of this band to that of the ligand indicates that it likely arises from the ¹π,π* transition of the acetylide ligand 35. This assignment is consistent with the minor solvent effect observed for this band, which is similar to that observed in the ligand. However, the bathochromic shift of this band compared to that of the ligand implies that there should be some delocalization of the ligand-centered molecular orbitals through the interactions with the platinum dπ orbitals. This notion of delocalized molecular orbitals is also supported by the lack of vibronic structure in this band, which is indicative of weak electron-vibronic coupling and is in line with a delocalized excited state.

TABLE 10
Photophysical parameters of F-15

| | λₑₘ/nm | | | λ_{T1-Tn}/nm |
|---|---|---|---|---|
| λ_{abs}/nm | (Φₑₘ; | | | (ε_{T1-Tn}/L · |
| (logε/L · | τ₀/μs; k_{Q}/L · | λₑₘ/nm | λ_{S1-Sn}/nm | mol⁻¹ · cm⁻¹; |
| mol⁻¹ · cm⁻¹)^{a} | mol⁻¹ · S⁻¹)^{b} | (τ/μs)^{c} | (τ_{S}/ps)^{d} | τ_{TA}/μs; Φ_{T})^{e} |
| 288 (4.55), | 565 (0.20; | 552 (289), | 606 | 620 (60170; |
| 374 (5.03), | 10.7; | 604 (273) | (145 ± 105) | 10.8; 0.14) |
| 423 (4.33) | 6.22 × 10⁸) | | | |

^{a}UV-vis absorption band maxima and molar extinction coefficients in CH₂Cl₂.
^{b}Emission band maximum, quantum yield, intrinsic lifetime, and self-quenching rate constant in CH₂Cl₂.
^{c}Emission band maxima and lifetimes in BuCN matrix at 77 K.
^{d}fs TA band maximum and singlet excited-state lifetime in CH₂Cl₂.
^{e}ns TA band maximum, triplet extinction coefficient, triplet excited-state lifetime and quantum yield in CH₂Cl₂.

In addition to the major band at 374 nm, a low-energy tail is observed between 410 nm and 500 nm in the spectrum of F-15. This band is red-shifted in less polar solvents, such as hexane and toluene, indicative of the charge-transfer nature of this band. With reference to that reported for other diimine platinum acetylides complexes, this band can be attributed to the ¹MLCT transition.

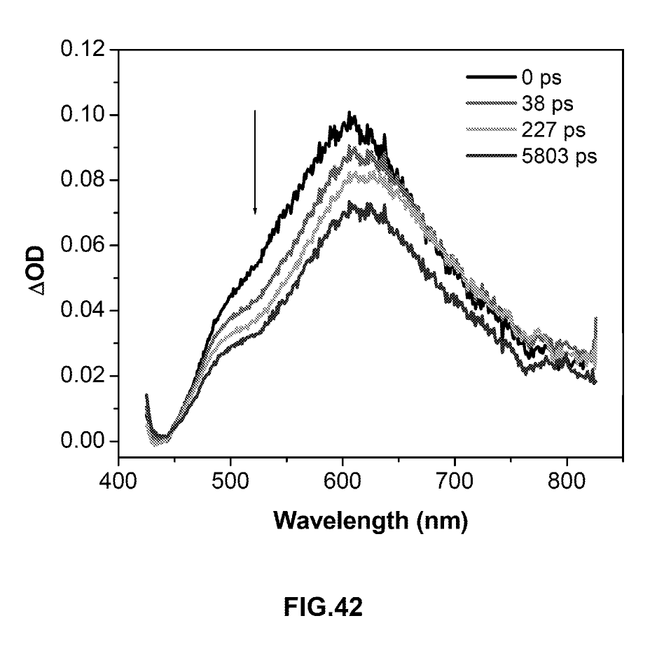

To understand the singlet excited-state absorption and obtain the lifetime of the singlet excited state, which will be used as an input parameter for fitting the Z-scan data, fs transient absorption measurements were carried out. The time-resolved TA spectrum of F-15 in CH₂Cl₂ is given in FIG. 42, which is measured using ultrafast femtosecond laser excitation (150 fs) at 400 nm. Immediately following the excitation pulse, the absorption occurs at 606 nm, which is similar to the S₁-Sₙ absorption band maximum (λₘₐₓ=623 nm) of the ligand 35 in benzene. Therefore, the transient absorption of F-15 right after the excitation could be tentatively assigned to the acetylide ligand centered ¹π,π* absorption. However, the somewhat different shapes of the two spectra imply that other excited states, likely the ¹MLCT state could also contribute to the spectrum of F-15. At longer delay time the band maximum bathochromically shifts to 620 nm, accompanied by an isosbestic point at 450 nm. This reflects the intersystem crossing from the ¹π,π*/¹MLCT states to the ³π,π*/³MLCT excited states. The spectrum at longer delay time is essentially the same as that measured by ns laser flash photolysis (shown in FIG. 41). Therefore, it is also attributed to the mixed ³π,π*/³MLCT absorption. The decay of the fs TA spectrum exhibits multi-exponential kinetics. The fast decay of 16.0±6.7 ps should be attributed to the internal conversion from the higher singlet excited state, the vibrational relaxation, and the solvent reorganization of the molecule. The lifetime of 145±105 ps is presumably assigned as the decay of the singlet excited state including intersystem crossing and internal conversion. A long lifetime that far exceeds the 6000 ps delay line of our experiment should be due to the decay of the triplet excited state. The singlet lifetime of F-15 is obviously much shorter than that of the acetylide ligand 35 (589 ps, measured in CH₂Cl₂), which should be attributed to the rapid intersystem crossing induced by the heavy-atom effect of platinum.

Z scan. Both the ns and fs transient absorption spectra indicate that complex F-15 exhibits relatively strong singlet and triplet excited-state absorption from 450 nm to 800 nm. However, the observed transient difference absorption is likely a combination of the excited-state absorption from the first excited state to the second excited state (S₁→S₂ or T₁→T₂) and from S₂ or T₂ to a higher excited state Sₙ or Tₙ (S indicates the singlet excited state and T refers to the triplet excited state). It is not possible to deconvolve these contributions using the transient absorption measurement alone. To obtain the absorption cross sections of the singlet and triplet excited states and separate the contributions from S₁ and S₂ states, open-aperture Z scans were carried out at 532 nm using both ns and ps laser pulses and at a variety of visible and near-IR wavelengths using ps laser pulses having a series of different energies. It is expected that at lower pulse energies only the lowest lying excited states will be populated, and the singlet excited-state absorption would be dominated by the absorption of S₁→S₂. At higher excitation energies, however, the S₂ state could acquire a significant population so that the absorption driving the transition S₂→Sₙ would make a non-negligible contribution to the Z-scan signal, which would have to be taken into account. At longer wavelengths where the ground-state absorption of F-15 is negligible, it is still possible to populate the excited state via two-photon absorption (TPA), so at these wavelengths two-photon absorption from the ground state in combination with subsequent excited-state absorption should be considered. To obtain values for the various absorption cross sections, the Z-scan experimental data were fitted by a five-level model that tracks the relative populations of the ground state S₀, and of the S₁, S₂, T₁, and Tₘ excited states, where Tₘ denotes a triplet excited state lying above T₁. The various photophysical parameters that appear in the model were determined from independent measurements: values of the ground-state absorption cross section σ₀(λ) were obtained from the UV-vis absorption spectrum; the singlet excited-state lifetime, from the decay of the fs TA; the triplet excited state lifetime, from the decay of ns TA; the triplet quantum yield, from the relative actinometry; and the effective triplet-triplet excited-state absorption cross section σ_{T}(λ) was deduced from the fs TA curve at 5.8-ns time delay in combination with the value of σ_{T} at 532 nm, 4.6×10⁻¹⁶ cm², which itself was determined from combined fitting of ns and ps Z-scan data.

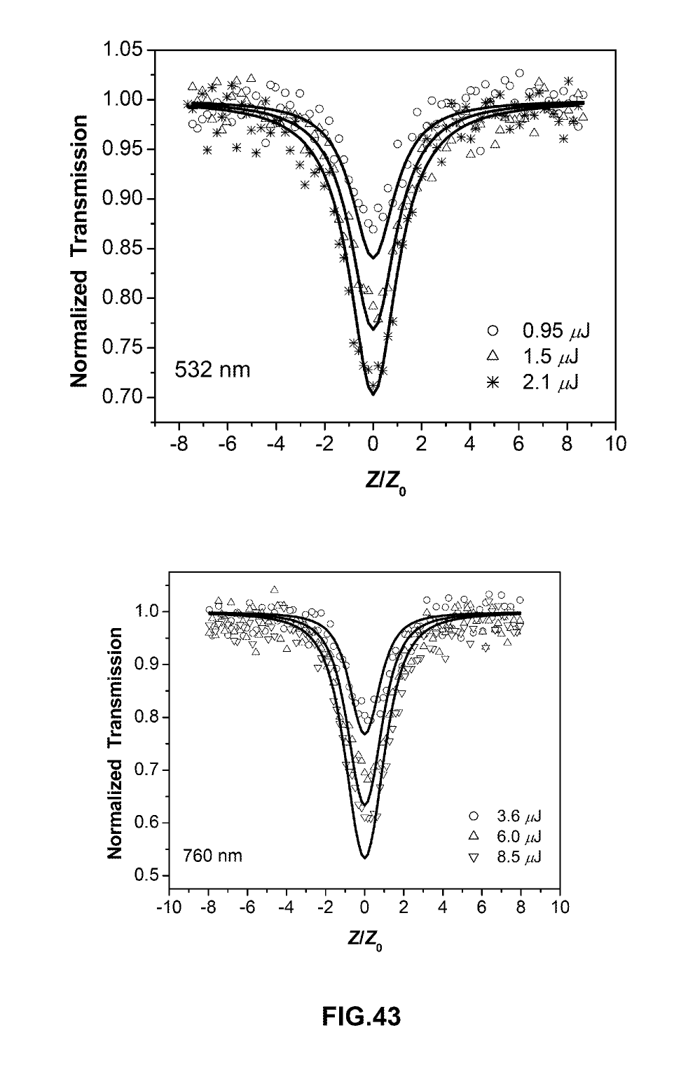

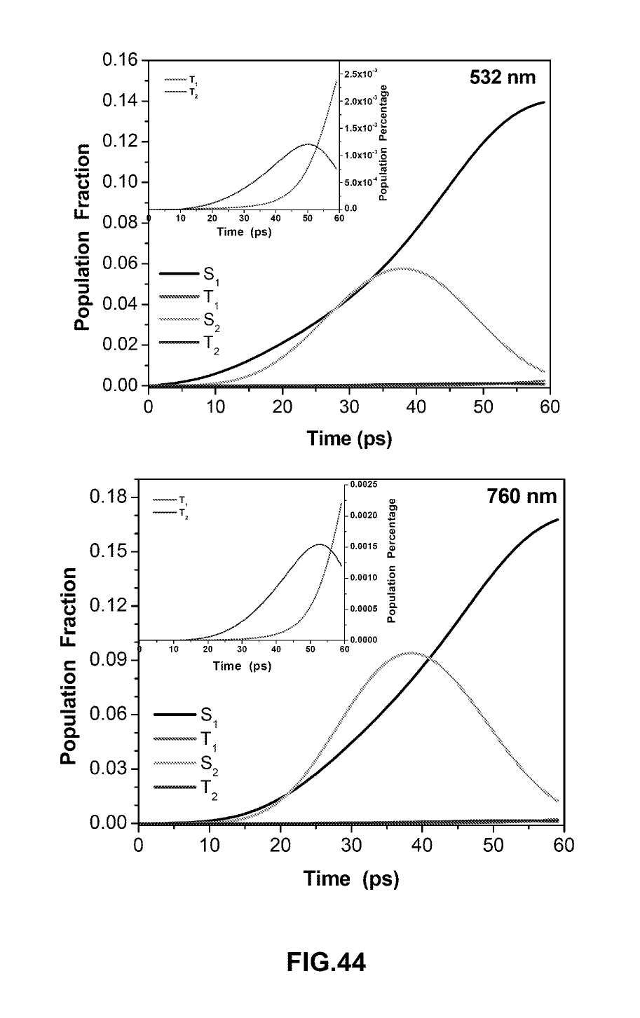

FIG. 43 shows representative Z scan experimental data and fitting curves for 1 at 532 nm and 760 nm at a series of different excitation energies. The resultant excited-state absorption cross sections and the two-photon absorption cross sections are displayed in Table 11. FIG. 44 shows the time evolution of the population densities of the affected excited states during the course of a representative Z-scan pulse. Below 600 nm, where the molecule has considerable ground-state absorption, both S₁ and S₂ have significant populations. Therefore the contributions resulting from absorption from both S₁ and S₂ were taken into account when fitting the Z scan data at these wavelengths. This gives rise to the cross sections shown in Table 11. At wavelengths greater than 600 nm, measurable ground-state absorption were not detected even in a saturated solution of 1 over a 10-mm path length. For this reason, in the Z scans conducted at wavelengths of 630 nm and above, the excited states are thought to be populated by two-photon absorption. Thus, at wavelength greater than 600 nm, the Z-scan signal manifests contributions not only from excited-state absorption, but from two-photon absorption as well. In order to deconvolve these contributions in the Z scans at wavelengths of 630 nm through 825 nm and so obtain values for σ₂(λ), it is thought that the estimated values of σ_{S}(λ) are derived from the measured value σ_{S}(532 nm)=6×10⁻¹⁷ cm² and the fs transient difference absorption spectrum at zero time delay (listed in Table 12) are dominated by the absorption from S₁ alone and thus accurately reflect the singlet excited-state absorption seen in the Z scans. In point of fact, the σ_{S} values obtained from the fs TA spectrum are actually effective cross sections that include contributions from both the absorption from S₁ and the absorption from S₂, though the relative importance of these contributions is unknown. In addition, the fs TA data in the range 780-825 nm are themselves intrinsically somewhat problematic, due to incomplete filtering of the 800-nm fundamental laser beam from the TA signal. Above 825 nm, the TA could not be measured because of the detection limit of our spectrometer, leaving no basis on which to estimate the singlet and triplet excited-state absorption cross sections at the wavelengths of 825-900 nm. Consequently, the two-photon absorption cross sections given in Table 12 for the wavelengths 825 nm through 900 nm inclusive should be considered as effective cross sections for excited-state-assisted two-photon absorption.

TABLE 11
Absorption cross sections of F-15 in CH₂Cl₂ solution^{a}

| λ | | σ_{S1}^{c} | σ_{T}^{d} | | | | | σ₂^{f} |
|---|---|---|---|---|---|---|---|---|
| NM | σ₀^{b} | 10⁻¹⁸ cm² | | σ_{S2}^{e} | σ_{S1}/σ₀ | σ_{T}/σ0 | σ_{S2}/σ₀ | GM |
| 450 | 39.4 | 35 | 94^{h} | 120 | 0.89 | 2.4 | 3.0 | — |
| 475 | 21.2 | 46 | 250^{h} | 70 | 2.2 | 11.8 | 3.3 | — |
| 500 | 5.55 | 48 | 375^{h} | 70 | 8.6 | 67.6 | 12.6 | — |
| 532 | 0.383 | 60 | 460 | 70 | 157 | 1.20 × 10³ | 183 | — |
| 550 | 0.0765 | 75 | 550^{h} | 70 | 980 | 7.19 × 10³ | 915 | — |
| 575 | 0.0188 | 95 | 760^{h} | 350 | 5.05 × 10³ | 4.04 × 10⁴ | 1.86 × 10⁴ | — |
| 600 | 0.0084 | 110 | 900^{h} | 1000 | 1.31 × 10⁴ | 1.07 × 10⁵ | 1.19 × 10⁵ | |
| 630 | ~0 | 100^{g} | 870^{h} | 30 | — | — | — | 1000 |
| 680 | ~0 | 70^{g} | 650^{h} | 30 | — | — | — | 400 |
| 740 | ~0 | 40^{g} | 400^{h} | 10 | — | — | — | 600 |
| 760 | ~0 | 34^{g} | 285^{h} | 10 | — | — | — | 1000 |
| 800 | ~0 | 28^{g} | 320^{h} | 10 | — | — | — | 300 |
| 825 | ~0 | 28^{g} | 240^{h} | 1 | — | — | — | 80 |
| 850 | ~0 | — | — | — | — | — | — | 600ⁱ |
| 875 | ~0 | — | — | — | — | — | — | 300ⁱ |
| 900 | ~0 | — | — | — | — | — | — | 300ⁱ |

^{a}Determined by fitting Z-scan data except where otherwise indicated.
^{b}The ground-state absorption cross section.
^{c}The first singlet excited-state absorption cross section.
^{d}The effective triplet excited-state absorption.
^{e}The second singlet excited-state absorption cross section.
^{f}The two-photon absorption cross section.
^{g}Determined from the value σ_{S}(532 nm) = 6 × 10⁻¹⁷ cm² and the fs transient difference absorption spectrum at 0 time delay.
^{h}σ_{T}(532 nm) = 4.6 × 10⁻¹⁶ cm² determined from combined fitting of ns and ps Z-scan data. For other wavelength, σ_{T}(λ) is determined from the value of σ_{T}(532 nm) and the fs transient difference absorption spectrum at 5.8-ns time delay.
ⁱEffective cross-section for excited-state-assisted two-photon absorption.

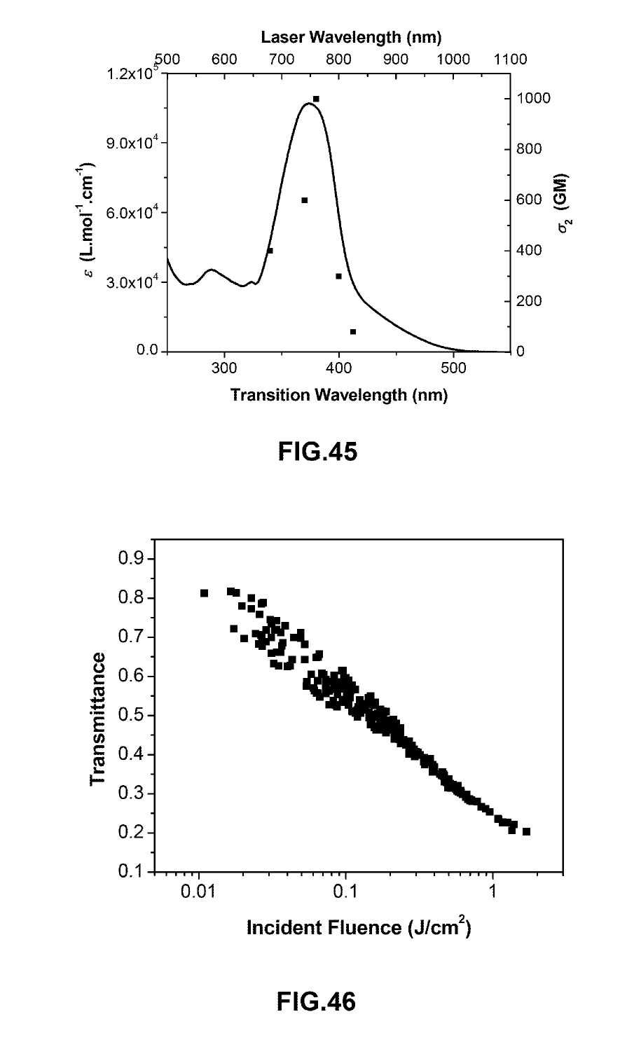

The excited-state absorption cross sections shown in Table 11 are all in the range of 10⁻¹⁷ cm² to 10⁻¹⁵ cm². These values are comparable to or even larger than those reported in the literature for other reverse saturable absorbers. Most importantly, due to the weak ground-state absorption of F-15 in the visible to the near-IR region, the ratios of the excited-state absorption cross section to that of the ground state become extremely large when the wavelength becomes longer. In addition to these large ratios, the two-photon absorption cross sections at the near-IR region deduced for F-15 are also the largest values reported for platinum complexes. It is also worth noting that the TPA band maximum of F-15 almost coincides with its corresponding one-photon absorption band maxima (FIG. 45), noting that the σ₂ values at 630, 850, 875 and 900 nm are not counted in plotting because the σ₂ value at 630 nm possibly has some contribution from one-photon absorption and the σ₂ values at 850-900 nm are effective TPA cross sections with contributions from both TPA and excited-state absorption). Because of the lack of central symmetry of this complex and the approximate overlap of the TPA peak with its one-photon absorption peak, it is reasonable to conclude that the lowest-energy TPA transition of F-15 corresponds to the S₀→S₁ transition.

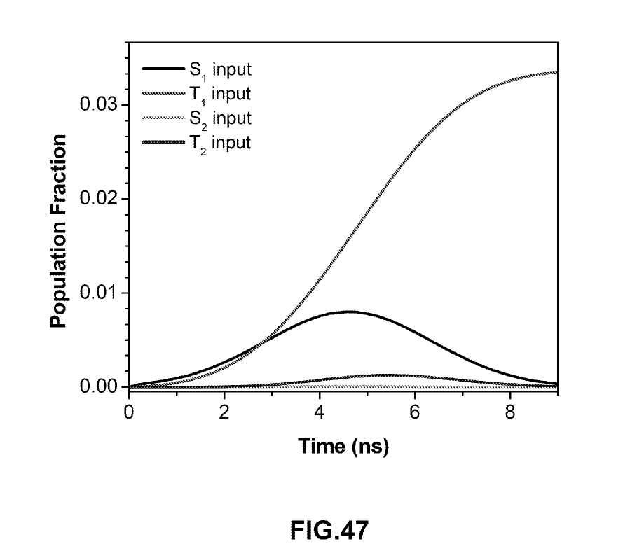

Reverse saturable absorption. The Z scan experiments and fitting results discussed above imply that complex F-15 could exhibit strong reverse saturable absorption in the visible spectral region due to the extremely large ratio of the excited-state absorption to that of the ground state. To demonstrate this, a nonlinear transmission experiment was carried out at 532 nm using 4.1 ns laser pulses. The result is shown in FIG. 46. It is obvious that at the lowest detectable incident fluence (˜0.01 J/cm²), the transmission already deviates from the linear transmission, indicating that the threshold for reverse saturable is equal to or smaller than 0.01 J/cm². With increased fluence, the transmission keeps decreasing. At the incident fluence of ˜1.6 J/cm², the transmission drops to 20%. This clearly manifests the reverse saturable absorption (RSA) at 532 nm. Referring to the population density shown in FIG. 47 for ns laser pulses at 532 nm, the observed RSA for ns laser pulse has contributions from both S₁ and T₁ absorption. The very large ratios of σ_{S1}/σ₀ and σ_{T}/σ₀ at 532 nm (shown in Table 11) lead to the strong reverse saturable absorption.

The platinum 2,2′-bipyridine complex bearing 2-(benzothiazol-2′-yl)-9,9-diethyl-7-ethynylfluorene ligands exhibits a strong absorption band at 374 nm in CH₂Cl₂ solution, which is attributed to the somewhat delocalized ¹π,π* transition of the acetylide ligands. A broad, weak ¹MLCT band appears between 410 and 500 nm. It is emissive at room temperature and at 77 K. The emitting state at room temperature can be switched from the acetylide ligand localized ³π,π* state in polar solvents, such as CH₃CN and CH₂Cl₂, to the ³MLCT state in less polar solvents such as hexane and toluene. The modulation of the order of the excited states by solvent polarity is supported by a transient absorption study using solvents of differing polarity. The most striking feature of complex F-15 is its very broad and strong nonlinear absorption in the visible to the near-IR region, which manifests itself in extremely large ratios of the excited-state absorption cross section to that of the ground-state in the visible spectral region and in the largest two-photon absorption cross sections in the near-IR region compared to the other reported platinum complexes. This feature, along with its weak ground-state absorption in the visible to the near-IR region, makes complex F-15 a very promising candidate for photonic devices that require large and broadband nonlinear absorption.

### Example 7

### Tuning Photophysics and Nonlinear Absorption of Bipyridyl Platinum(II) Bisstilbenzylacetylide Complexes by Auxiliary Substituents

Synthesis and Characterization. All of the reagents and solvents for synthesis were purchased from Aldrich Chemical Co. or Alfa Aesar and used as is unless otherwise stated. Silica gel is from sorbent technology in standard grade (60 Å, 230-400 mesh, 500-600 m²/g, pH: 6.5-7.5). The complexes F-16-F-21 were characterized by ¹H NMR, electrospray ionization mass spectrometry (ESI-MS), and elemental analyses. The ligands 38-41 were characterized by ¹H NMR and elemental analyses. Every intermediate is characterized by ¹H NMR. ¹H NMR was obtained on Varian Oxford-VNMR spectrometers at the frequencies of 300 M, 400 M, or 500 M. ESI-MS analyses were performed at a Bruker BioTOF III mass spectrometer. Elemental analyses were carried out by NuMega Resonance Laboratories, Inc. in San Diego, Calif.

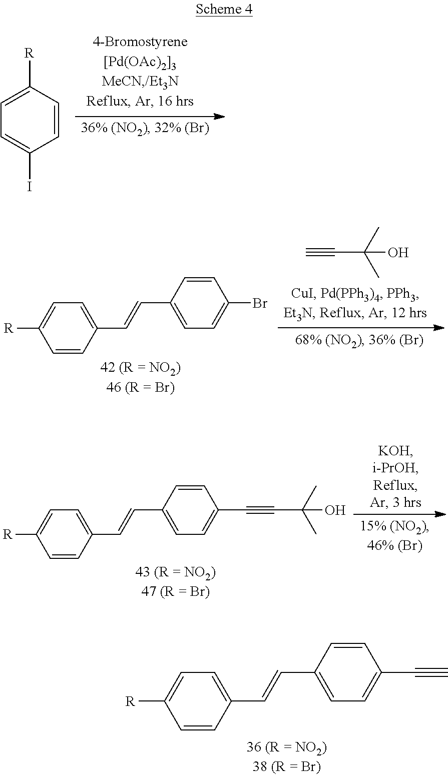

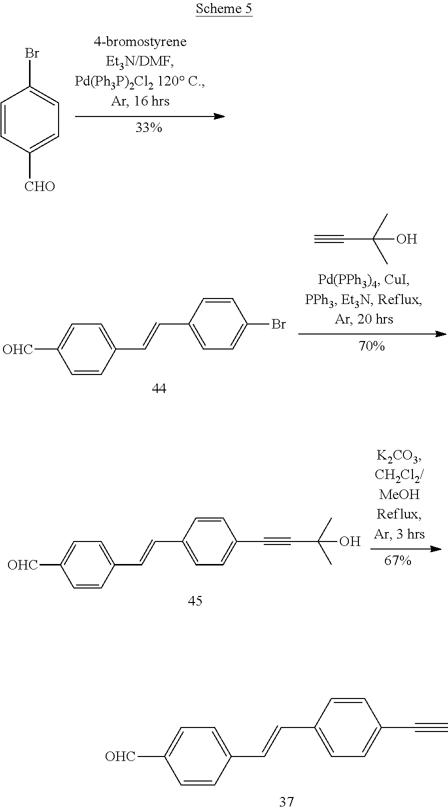

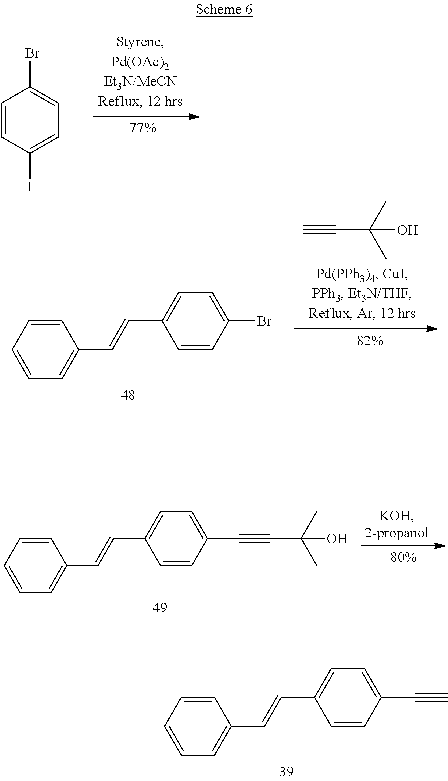

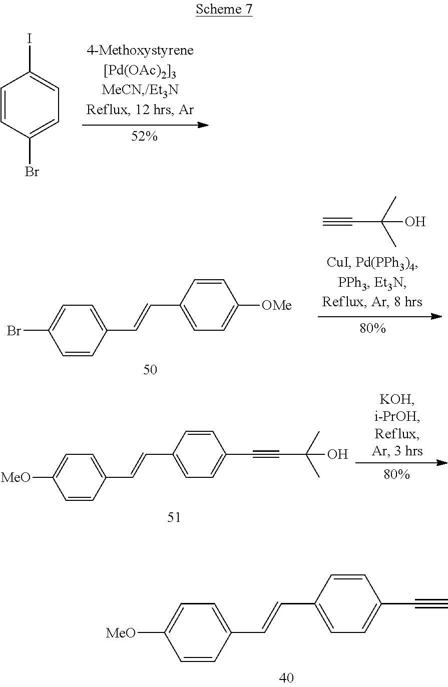

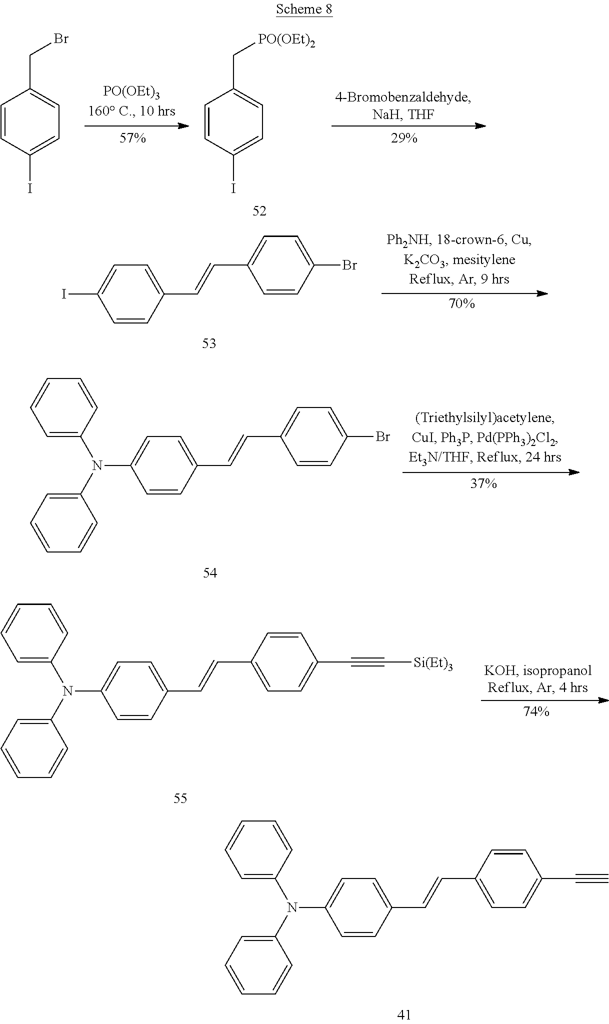

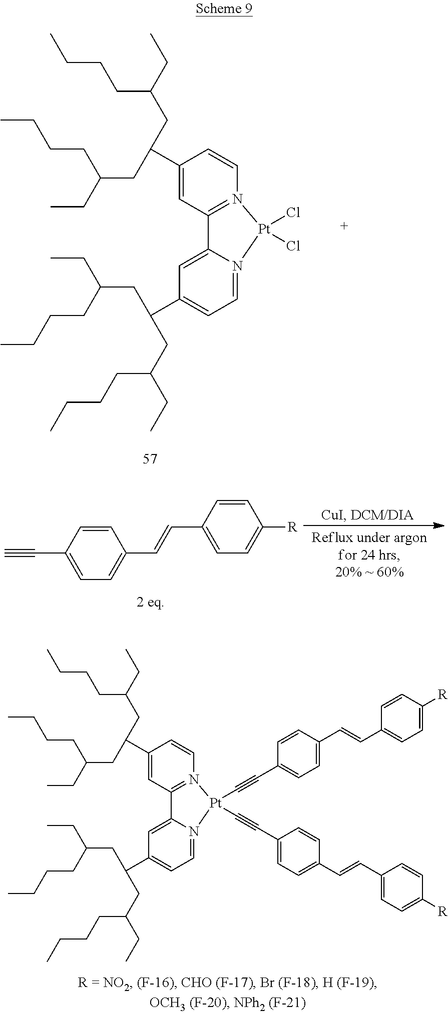

4,4′-Di(5,9-diethyl-7-tridecanyl)-2,2′-bipyridine (56) was synthesized following literature procedure. Domínguez-Gutíerrez, D.; De Paoli, G.; Guerrero-Martínez, A.; Ginocchietti, G.; Ebeling, D.; Eiser, E.; De Cola, L.; Cornelis J.; Elsevier, C. J. J. Mater. Chem. 2008, 18, 2762 (incorporated herein by reference). 57 was synthesized by the reaction of K₂PtCl₄ with 56 in refluxing aqueous HCl solution. 42, 44, 46, 48 and 50 were synthesized by Heck reaction. 53 was synthesized by Wittig reaction. 4 was synthesized by Ullmann reaction from 53. Sonogashira coupling reaction of 42, 44, 46, 48, 50, and 54 with ethynyltrimethylsilane or 2-methyl-3-buytn-2-ol followed by hydrolysis by treating with K₂CO₃ or KOH in i-PrOH afforded ligands 36-41.

56. Colorless oil. 1.13 g, yield: 89%. ¹H NMR (400 M Hz, CDCl₃): 8.55 (d, J=4.8 Hz, 2H), 8.23 (s, 2H), 7.08 (dd, J=5.2, 1.6 Hz), 2.80 (m, 2H), 1.42-1.62 (m, 8H), 0.98-1.40 (m, 36H), 0.70-0.90 (m, 24H).

57. Yellow solid. 1.03 g, yield: 64%.¹H NMR (400 M Hz, CDCl₃): 0.72-0.87 (m, 24 H), 0.92-1.37 (m, 36H), 1.55 (m, 8H), 7.32 (dd, J=6, 1.6 Hz, 2H), 7.60 (s, 2H), 9.64 (d, J=6 Hz, 2H).

General procedure for synthesis of complexes F-16-F-21. The mixture of 57 (0.1 mmol), stilbenzylacetylide ligand (0.24 mmol), CuI (5 mg), CH₂Cl₂ (15 mL), and diisopropylamine (5 mL) was refluxed under argon for 24 hrs. After reaction, the excess ligands were removed by flash silica gel column eluting with dichloromthane. The collected yellow or red solid was recrystallized from dichloromethane and hexane.

F-16. Red powder, 60 mg, yield: 49%. ¹H NMR (CDCl₃, 400 MHz): 9.57 (s, 2H), 8.17-8.20 (m, 4H), 7.72 (s, 2H), 7.59-7.61 (m, 8H), 7.39-7.46 (m, 6H), 7.23 (d, J=16.4 Hz, 2H), 7.08 (d, J=16.4 Hz, 2H), 2.92 (m, 2H), 1.56 (m, 8H), 1.05-1.38 (m, 36H), 0.75-0.89 (m, 24H). ESI-HRMS: m/z Calc. for [C₇₆H₉₆N₄O₄Pt+Na]⁺: 1347.6994; Found: 1347.6966. Anal. Calc. for C₇₆H₉₆N₄O₄Pt.2H₂O.CH₂Cl₂: C, 63.97; H, 7.11; N, 3.88; Found: C, 63.96; H, 7.61; N, 4.20.

F-17. Red powder, 72 mg, yield: 55%. ¹H NMR (CDCl₃, 400 MHz): 9.96 (s, 2H), 9.65 (s, 2H), 7.84 (d, J=8.4 Hz, 4H), 7.71 (s, 2H), 7.62 (d, J=8.4 Hz, 4H), 7.55 (d, J=8.0 Hz, 2H), 7.43 (d, J=8.0 Hz, 2H), 7.39 (d, J=5.6 Hz, 2H), 7.23 (d, J=16.4 Hz, 2H), 7.08 (d, J=16.4 Hz, 2H), 2.91 (m, 2H), 1.57 (m, 8H), 1.04-1.40 (m, 36H), 0.75-0.89 (m, 24H). ESI-HRMS: m/z Calc. for [C₇₆H₉₈N₂Pt+Na]⁺: 1312.7174; Found: 1312.7166. Anal. Calc. for C₇₈H₉₈N₂O₂Pt.2H₂O: C, 70.40; H, 8.03; N, 2.11; Found: C, 70.29; H, 8.22; N, 2.48.

F-18. Red powder, 57 mg, yield: 41%. ¹H NMR (CDCl₃, 400 MHz): 9.69 (d, J=5.6 Hz, 2H), 7.69 (s, 2H), 7.31-7.53 (m, 18H), 7.05 (d, J=16.4 Hz, 2H), 6.96 (d, J=16.4 Hz, 2 H), 2.90 (m, 2H), 1.57 (m, 8H), 1.05-1.36 (m, 36H), 0.75-0.89 (m, 24H). ESI-HRMS: m/z Calc. for [C₇₆H₉₆N₂Br₂Pt+Na]⁺: 1415.5478; Found: 1415.5461. Anal. Calc. for C₇₆H₉₆Br₂N₂Pt: C, 65.55; H, 6.95; N, 2.01; Found: C, 65.59; H, 7.17; N, 2.34.

F-19. Yellow powder, 26 mg, yield: 21%. ¹H NMR (CDCl₃, 400 MHz): 9.71 (d, J=5.6 Hz, 2H), 7.69 (s, 2H), 7.48-7.54 (m, 8H), 7.31-7.42 (m, 10H), 7.19-7.24 (m, 2H), 7.02 (d, J=16.4 Hz, 2H), 7.09 (d, J=16.4 Hz, 2H), 2.91 (m, 2H), 1.53-1.58 (m, 8H), 1.05-1.38 (m, 36H), 0.75-0.90 (m, 24H). ESI-HRMS: m/z Calc. for [C₇₆H₉₈N₂Pt+Na]⁺: 1256.7276; Found: 1256.7227. Anal. Calc. for C₇₆H₉₈N₂Pt.0.5C₆H₁₄: C, 74.26; H, 8.28; N, 2.19; Found: C, 74.53; H, 8.40; N, 2.37.

F-20. Yellow powder, 78 mg, yield: 60%. ¹H NMR (CDCl₃, 400 MHz): 9.76 (d, J=6.0 Hz, 2H), 7.72 (m, 2H), 7.41-7.56 (m, 12H), 6.98 (d, J=16 Hz, 2H), 7.05 (d, J=16 Hz, 2 H), 6.92 (d, J=9.0 Hz), 3.86 (s, 6H), 2.95 (m, 2H), 1.50-1.73 (m, 8H), 1.09-1.46 (m, 36H), 0.70-0.99 (m, 24H). ESI-HRMS: m/z Calc. for [C₇₈H₁₀₂N₂O₂Pt+Na]⁺: 1316.7487; Found: 1316.7515. Anal. Calc. for C₇₈H₁₀₂N₂O₂Pt: C, 72.36; H, 7.94; N, 2.16; Found: C, 71.87; H, 8.25; N, 2.29.

F-21. Red powder, 71 mg, yield: 45%. ¹H NMR (CDCl₃, 400 MHz): 9.69 (m, 2H), 7.69 (s, 2H), 7.51 (d, J=8.4 Hz, 2H), 7.35-7.38 (m, 10H), 7.21-7.26 (m, 8H), 7.07-7.10 (m, 8H), 6.97-7.04 (m, 12H), 2.91 (m, 2H), 1.58 (m, 8H), 1.05-1.41 (m, 36H), 0.75-0.90 (m, 24H). Anal. Calc. for C₁₀₀H₁₁₆N₄Pt.0.5CH₂Cl₂: C, 74.90; H, 7.32; N, 3.48; Found: C, 75.20; H, 7.72; N, 3.74.

### Photophysical Measurements

The solvents for photophysical experiments that are purchased from VWR International are spectroscopic grade and used as is without further purification. An Agilent 8453 spectrophotometer was used to record the UV-vis absorption spectra in different solvents. A SPEX fluorolog-3 fluorometer/phosphorometer was used to record the steady-state emission spectra in different solvents. The emission quantum yields were determined by the relative actinometry method¹⁴ in degassed solutions, in which a degassed 1 N sulfuric acid solution of quinine bisulfate (Φₑₘ=0.546, λₑₓ=347.5 nm) was used as the reference. The femtosecond transient absorption measurements were performed using a femtosecond pump-probe UV-vis spectrometer (HELIOS) manufactured by Ultrafast Systems LLC. The sample solution in a 2 mm cuvette was excited at 400 nm using a 150 fsTi:Sapphire laser (Spectra Physics Hurricane, 1 kHz repetition rate, 1 mJ/pulse at 800 nm), and the absorption was probed from 425 to 800 nm with sapphire generated white-light continuum. The emission lifetime and the triplet transient difference absorption (TA) spectrum and the decay time were measured in degassed solutions on an Edinburgh LP920 laser flash photolysis spectrometer. The third harmonic output (355 nm) of a Nd:YAG laser (Quantel Brilliant, pulsewidth 4.1 ns, repetition rate was set at 1 Hz) was used as the excitation source. Each sample was purged with argon for 30 min before each measurement. The triplet excited-state absorption coefficient (ε_{T}) at the TA band maximum was determined by the singlet depletion method.¹⁶ The following equation was used to calculate the ε_{T}.

$${ɛ}_{T} = {ɛ}_{S} * \frac{Δ {OD}_{T}}{Δ {OD}_{S}}$$

where ΔOD_{S} is minimum of the bleaching band and ΔOD_{T} is the maximum of the absorption band in the TA spectrum, and εₛ is the ground-state molar extinction coefficient at the wavelength of the bleaching band minimum. After the ε_{T} value is obtained, the Φ_{T} could be obtained by the relativeactinometry, in which SiNc in benzene was used as the reference (ε₅₉₀=70000 M⁻¹ cm⁻¹, Φ_{T}=0.20).

### Nonlinear Transmission

The nonlinear absorption of complex F-15-F-21 was characterized by nonlinear transmission experiment at 532 nm using a ns laser. The nonlinear transmission experiment was conducted in CH₂Cl₂ in a 2-mm cuvette using 4.1 ns laser pulses at 532 nm. The light source was a Quantel Brilliant ns laser with a repetition rate of 10 Hz. The experimental setup and details are the same as previously described. Guo, F.; Sun, W.; Liu, Y.; Schanze, K. S. Inorg. Chem. 2005, 44, 4055 (incorporated herein by reference). A 40-cm plano-convex lens was used to focus the beam to the 2-mm thick sample cuvette. The linear transmission of the solution was adjusted to 80% at 532 nm.

### Results

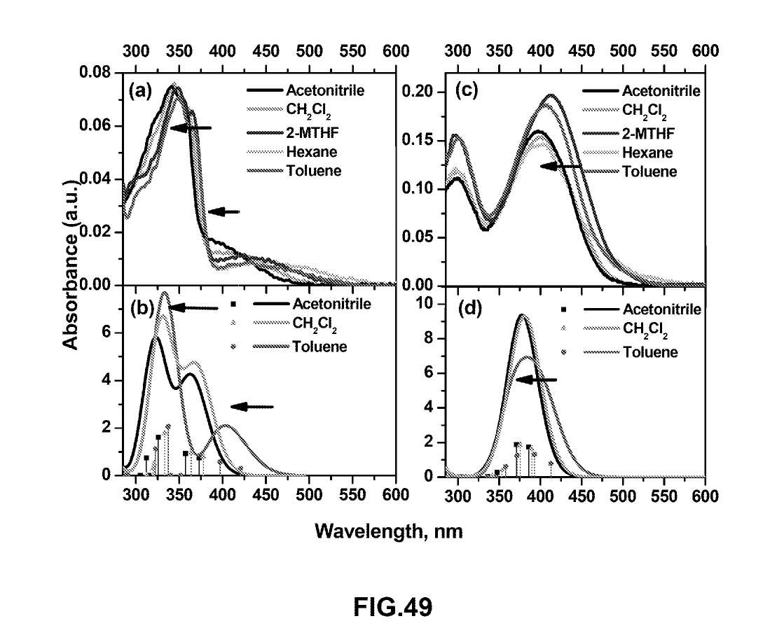

Electronic absorption. The UV-Vis spectra of complexes F-16-F-21 are shown in FIG. 49. The absorption of all complexes obeys Lambert-Beer's law in the concentration range studied (1×10⁻⁶ mol/L to 1×10⁻⁴ mol/L), indicating that no ground state absorption occurs in the concentration range studied.

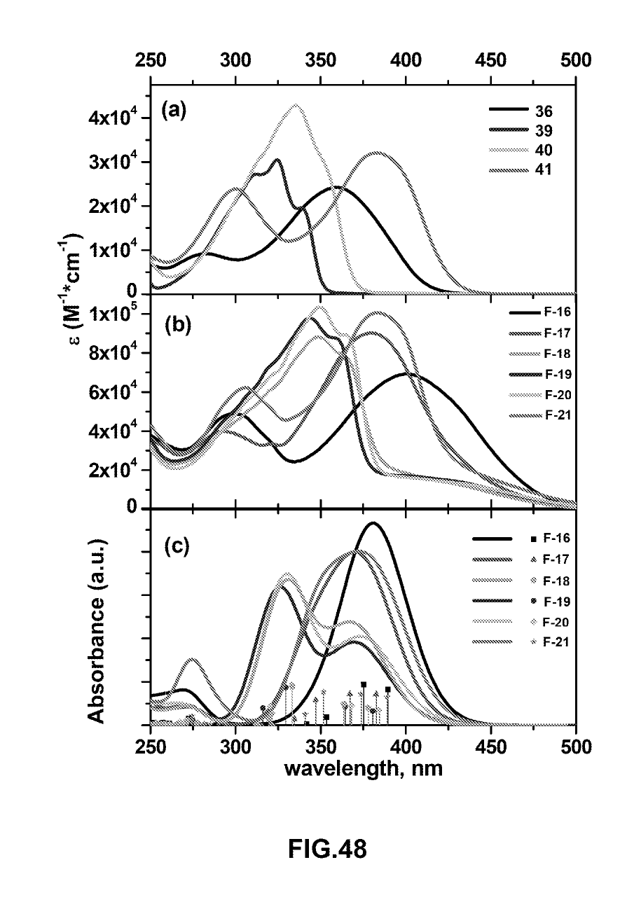

The absorption spectra of complexes F-16, F-19, F-20, and F-21 (FIG. 48b) in the region between 300 and 375 nm resemble those of their corresponding stilbenzylacetylide ligands (shown in FIG. 48a.), indicating that the absorption arises from the ligand-centered ¹π,π* transitions. The red-shift of the spectra indicates the delocalization through the dπ orbital of platinum. At the wavelength longer than 380 nm, a shoulder that is absent in the ligands absorption spectra is observed. Compared to the bands centered around 340-350 nm, this shoulder shows significant solvatochromic effect (as illustrated in FIG. 49a), implying the charge transfer nature of this shoulder.

TABLE 13
Absorption parameters for complexes F-16-F-21 and ligands 36, 39, 40,
and 41.

| | λ_{abs}/nm (εₘₐₓ/M⁻¹ cm⁻¹) | ^{Theor}λ_{abs}/nm (ex. state) | f_{osc} |
|---|---|---|---|
| F-16 | 401 (69325) | 389 (S1) | 1.6529 |
| | 298 (48900) | 375 (S2) | 1.8782 |
| | | 353 (S3) | 0.3870 |
| F-17 | 380 (90250) | 383 (S1) | 1.4325 |
| | 292 (40075) | 367 (S2) | 1.4423 |
| | | 347 (S3) | 1.1505 |
| F-18 | 420 (14575) | 378 (S1) | 0.811 |
| | 363 (78725) | 364 (S2) | 0.9787 |
| | 349 (88275) | 333 (S3) | 1.7741 |
| | 333 (74500) | | |
| F-19 | 410 (16475) | 381 (S1) | 0.6476 |
| | 360 (87300) | 364 (S2) | 0.8692 |
| | 344 (97850) | 329 (S3) | 1.7507 |
| | 325 (77375) | | |
| F-20 | 415 (14975) | 384 (S1) | 0.7001 |
| | 365 (89250) | 368 (S2) | 0.9100 |
| | 349 (103475) | 333 (S3) | 1.8807 |
| | 335 (87225) | | |
| F-21 | 385 (100475) | 389 (S1) | 1.3019 |
| | 305 (62125) | 374 (S2) | 1.4375 |
| | | 352 (S3) | 1.5416 |
| 36 | 360 (24263) | | |
| 39 | 325 (30500) | | |
| 40 | 336 (42850) | | |
| 41 | 384 (32075) | | |

λ_{abs}—absorption wavelength,
εₘₐₓ—extinction coefficient,
^{Theor}λ_{abs}—calculated wavelength corresponding to the transition between the ground and excited state of interest (number of excited state is shown in parentheses),
f_{osc}—calculated oscillator strength for the corresponding excitations.

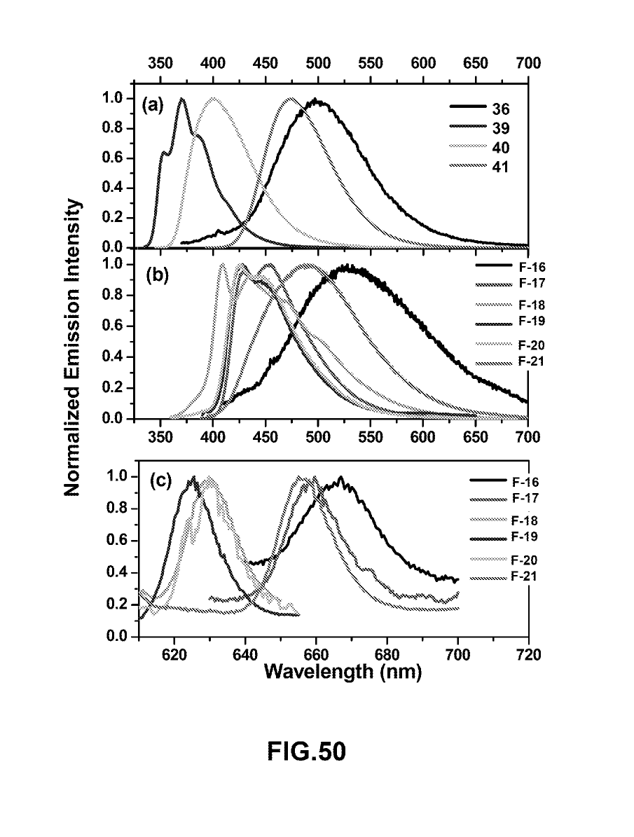

Photoluminescence. All of the complexes exhibit weak emission both at room temperature in dichloromethane solution and at 77 K in butyronitrile glassy matrix. The lifetime of the emission could not be detected by our spectrometer due to either too short lifetime or too weak signal. The emission spectra of the complexes F-16-F-21 at room temperature are shown in FIG. 50, and the emission data is summarized in Table 14. For all complexes, the Stokes shifts of the emission at room temperature are less than 80 nml. This feature along with the short lifetimes suggests that the emission of these complexes at room temperature originates from the singlet excited state. At 77 K, weak phosphorescence was observed between 625 nm and 667 nm. The emission properties of the complexes were further studied in various solvents at room temperature. for Complexes F-16 and F-21 were found to exhibit significant solvatochromic effect, while complexes F-17, F-18, F-19, and F-20 are insensitive to solvent polarity.

To elucidate the nature of emission of complexes F-16-F-21, their spectra are compared to the photoluminescence of the corresponding ligands, 36, 39, 40, and 41 (FIG. 50a). The emission spectra of complexes F-16 and F-21 very closely represent emission of their ligands, slightly shifted to the red, which is indicative of the predominant intraligand character of the photoluminescence in this range. All other complexes emit at substantially lower energy compared to their corresponding ligands, which indicates a strong delocalization induced by platinum dπ orbitals, and significant contribution from MLCT state. The emission of 39 can be assigned to ¹π,π* transition by comparing with the trans-stilbene emission from the literature. 40 emits at a similar level as 39 while the 36 and 41 spectra are significantly red-shifted due to presence of the strong electron accepting/donating groups, repsectively.

TD-DFT calculations of the singlet and triplet emission support this assignment and show that in complexes 1 and 6 the singlet emission stems from the dissymmetric ¹π,π* transition in one of the single stilbenzylacetylide ligands, which become slightly twisted relative to the Pt coordination plane. In all other complexes this character is strongly mixed with the ¹MLCT from Pt to bPy moiety. Triplet state emission follows the same scheme as the singlet emission, but involves a more symmetric ³π,π* excitation delocalized over both stylbenilacetylide ligands. Overall, calculated trends in singlet and triplet emission are very close to the trends observed by experiments (Table 14).

The nature of red-shift in the photoluminescence spectra observed for the complexes with the stronger electron donating/accepting groups (F-16 and F-21) can be explained by the interplay in the electronic levels of the stylbenzyl ligands and the MLCT states, similar to the picture discussed in the absorption part. Strong electron donating/accepting groups lower the energies of the stylbenilacetylide π,π* transitions relative to the MLCT states, so that the lowest excited states bear more of the π,π* character. This process is highly sensitive to the solvent polarity, as solvent can partially stabilize the dipole moment induced by the substituent group and raise the intraligand transition energies. In other words, in a less polar solvent, π,π* transitions have more admixture of MLCT character.

TABLE 14
Photoemission properties

| | | λ_{T}₁-Tₙ/nm (τ_{TA}/ns, | | |
|---|---|---|---|---|
| | λₑₘ/nm | ε_{T}₁-Tₙ/M⁻¹ | ^{theor}λ_{fluo}/ | [symbol] (^{theor} [symbol] λₚₕₒₛ/ |
| | (Φₑₘ) | cm⁻¹, Φ_{TA}) | nm | nm |
| F-16 | 530 (—) | 740 (401, 98760, 0.17) | 497 | 663 |
| F-17 | 454 (—) | 510 (225, 184760, | 472 | |
| | | 0.079) | | |
| F-18 | 409 (—) | 435 (48, —, —) | 449 | 638 |
| F-19 | 425 (—) | 460 (64, —, —) | 452 | 638 |
| F-20 | 428 (—) | 460 (73, —, —) | 460 | |
| F-21 | 492 (—) | 520 (198, 235514, | 463 | 668 |
| | | 0.075) | | |
| 36 | 499 (0.007) | 435 (—, —, —) | | |
| 39 | 370 (0.10) | 505 (—, —, —) | | |
| 40 | 401 (0.03) | 510 (—, —, —) | | |
| 41 | 474 (0.58) | 470 (—, —, —) | | |

[symbol]

indicates data missing or illegible when filed

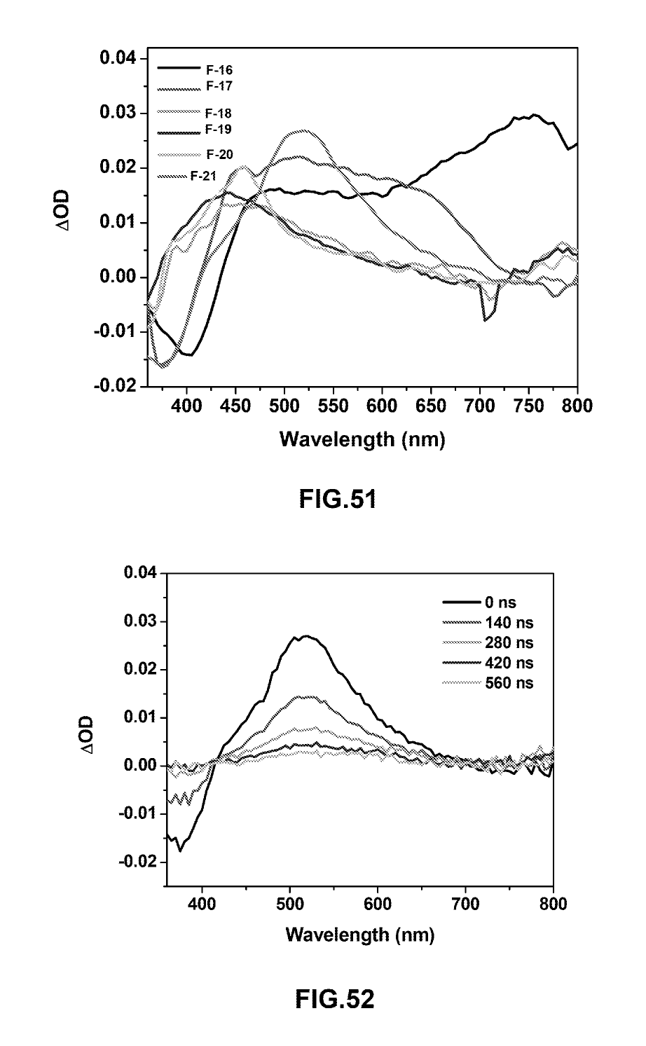

Transient difference absorption. The nanosecond and femtosecond transient difference absorption (TA) for complexes F-16-F-21 and nanosecond TA for ligand 36, 39, 40 and 41 were all measured. Through this TA experiment, the scope of the reverse saturate absorption (RSA) can be determined. The nanosecond TAs of complexes F-16-F-21 in acetonitile solution at zero time decay were shown in FIG. 51. All of the complexes have strong TA signals that are time-resolved (illustrated by the time resolved decay of F-21 in FIG. 52). These absorptions are all enhanced compared to their corresponding ligands, indicating enhanced intersystem crossing (ISC) induced by the heavy-atom effect. Complexes F-16, F-17, and F-21 show especially wide ranged and strong transient absorptions, suggesting they might serve as good nonlinear transmission materials due to strong RSA in their TA range. The ground-state absorption of F-16, F-17, and F-21 were observed as bleaching bands in the corresponding range. The transient absorption spectra of the complexes all resemble those of their ligands except complex F-16, indicating TAs of these complexes are from the excited stated localized in the ligands. The lifetime and molar extinction coefficient of F-16, F-17 and F-21 are determined and listed in Table 14.

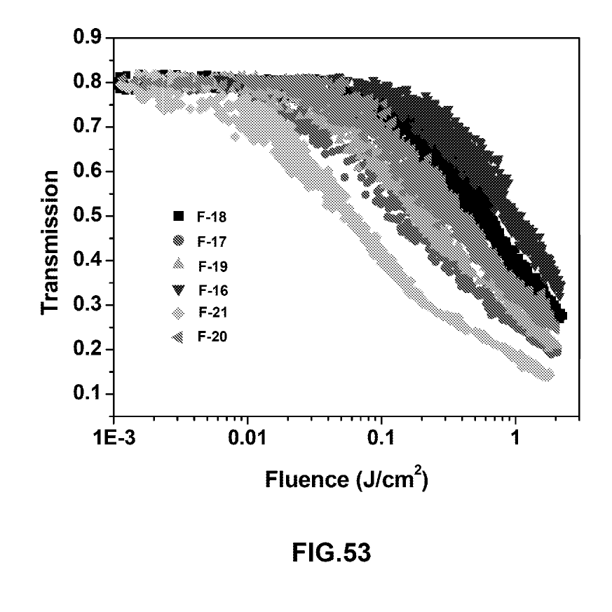

Nonlinear transmission. The nonlinear transmission of the complexes F-16-F-21 for ns 532 nm laser pulses was studied in dichloromethane solution at a linear transmittance of 80%. The results are shown in FIG. 53. All of the complexes show good to excellent nonlinear transmission, among which F-21 exhibits the strongest nonlinear transmission. The nonlinear transmission performance of the complexes increases in the order of F-16 <F-18 <F-20 <F-19 <F-17 <F-21. The excellent nonlinear transmission performance of complexes F-21 and F-17 can be attributed to their large ratios of the excited-state absorption relative to their ground-state absorption at 532 nm.

### PROPHETIC EXAMPLES

### Example 8

### Optical-Switching Devices

The metal complexes synthesized in the above examples will be used in optical-switching devices. In forming the optical-switching device, the metal complexes will be dissolved in a solvent, and the resulting solution will be substantially filling the cavity between the transparent substrates of the optical-switching device.

### Example 9

### Organic Light-Emitting Diodes

The metal complexes and ligands synthesized in the above examples will be used as light-emitting materials in an organic light-emitting diode (OLED). Layers of indium tin oxide, the organic light-emitting materials, and aluminum will be continuously deposited in a vacuum chamber on a glass substrate. The indium tin oxide will be used as an anode, and the aluminum layers will be used as a cathode. The organic compound layer of light-emitting materials is thus interposed between the anode and the cathode. A DC voltage will be applied to the OLED with the anode as a positive electrode and the cathode as a negative electrode, as result of which light will be emitted.

### Example 10

### Chemical Sensors

The metal complexes and ligands synthesized in the above Examples will be used as chemical sensors. Filter paper strips will be impregnated with a coating solution that includes the metal complexes. The coated filter paper strips will be dried. The dried filter paper strips will be contacted with organic vapors, and the color of the filter paper strips before the exposure to the organic vapors will be compared to that of the filter paper strips after the exposure.

### Example 11

### Anion Sensors

The metal complexes and ligands synthesized in the above examples will be used as anion sensors. They are sensitive to basic anions such as F⁻, H₂PO₄⁻ and OAc⁻. Upon addition of F⁻, H₂PO₄⁻, or OAC⁻ to the DMSO solutions of the ligand or complexes, the color of the solutions will change drastically from yellow to purple or blue; whereas addition of NO₃⁻, Cl⁻, Br⁻ and I⁻ (all as tetra-n-butylammonium salts, TBA salts) has no effect on the solution color. Addition of F⁻, H₂PO₄⁻ or OAC⁻ into the DMSO solutions of the ligands or complexes also induces substantial changes in their respective emission spectrum.

What is claimed is:

1. A ligand of formula (I): 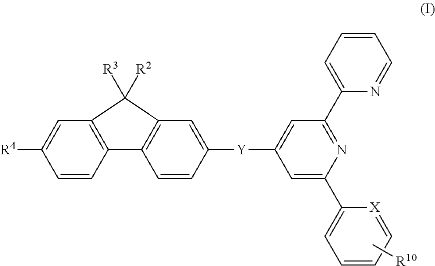
wherein:
R¹⁰ is H or —OR^{a};
R² and R³ are each independently C₁-C₂₄ alkyl, C₂-C₂₄ alkenyl, C₂-C₂₄ alkynyl or —(O—CH₂—CH₂)ₙ—OCH₃;
R⁴ is H, halo, aryl, heterocyclyl, arylalkynyl, heterocyclylalkyl, arylalkynyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f};
X is C or N;
each n is independently an integer from 1-12;
each R^{a}, R^{b}, R^{c}, R^{d}, R^{e}, and R^{f} is independently selected from H, C₁-C₂₄ alkyl, C₂-C₂₄ alkenyl or C₂-C₂₄ alkynyl, aryl, heterocyclyl, arylalkyl, and heterocyclylalkyl; and
Y is a bond, —CH═CH—, —CH═CH-Ph-, or —C≡C—;
wherein when the ligand of formula (I) bears a charge, it further comprises one or more counterions.

2. An organic light-emitting diode, comprising:
an anode;
a cathode; and
an organic compound layer interposed between the anode and the cathode, wherein the organic compound layer includes a ligand having the formula of claim 1.

3. A chemical sensor comprising:
a fibrous substrate; and
a coating solution including a ligand impregnated to the substrate, the ligand having the formula of claim 1.

4. A ligand of formula (II): 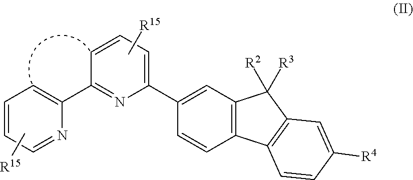
wherein:
the dashed line represents the presence or absence of an optionally substituted aromatic ring or fused aromatic rings;
R² and R³ are each independently C₁-C₂₄ alkyl, C₂-C₂₄ alkenyl, C₂-C₂₄ alkynyl or —(O—CH₂—CH₂)ₘ—OCH₃;
R⁴ is H, halo, aryl, heterocyclyl, arylalkynyl, arylalkyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl, or —CH═N—NH—R^{f};
each R¹⁵ is independently selected from H, halo, aryl, heterocyclyl, arylalkynyl, C₁-C₂₄ alkyl, C₂-C₂₄ alkenyl, or C₂-C₂₄ alkynyl;
each m is independently an integer from 1-12; and
each R^{b}, R^{c}, R^{d}, R^{e}, and R^{f} is independently selected from H, C₁-C₂₄ alkyl, C₂-C₂₄ alkenyl, C₂-C₂₄ alkynyl, heterocyclyl, arylalkyl, heterocyclylalkyl and aryl,
wherein when the ligand of formula (II) bears a charge, it further comprises one or more counterions.

5. An organic light-emitting diode, comprising:
an anode;
a cathode; and
an organic compound layer interposed between the anode and the cathode, wherein the organic compound layer includes a ligand having the formula of claim 4.

6. A chemical sensor comprising:
a fibrous substrate; and
a coating solution including a ligand impregnated to the substrate, the ligand having the formula of claim 4.

7. A ligand of formula (III): 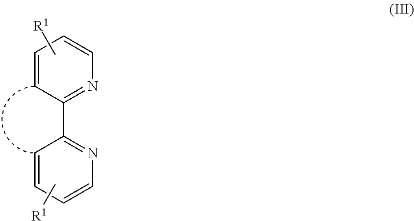
wherein:
each R¹ is independently a group of the following formula: 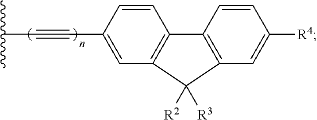
each n equals to 0 or 1;
each R² and R³ is independently C₁-C₂₄ alkyl, C₂-C₂₄ alkenyl or C₂-C₂₄ alkynyl, or —(O—CH₂—CH₂)ₘ—OCH₃;
each R⁴ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f}; and
each R^{b}, R^{c}, R^{d}, R^{e}, and R^{f} is independently selected from H, C₁-C₂₄ alkyl, C₂-C₂₄ alkenyl, C₂-C₂₄ alkynyl, heterocyclyl, arylalkyl, heterocyclylalkyl and aryl;
each m is independently an integer from 1-12; and
wherein the dashed line represents the presence or absence of an optionally substituted aromatic ring or fused aromatic rings; and
wherein when the ligand of formula (III) bears a charge, it further comprises one or more counterions.

8. An organic light-emitting diode, comprising:
an anode;
a cathode; and
an organic compound layer interposed between the anode and the cathode, wherein the organic compound layer includes a ligand having the formula of claim 7.

9. A chemical sensor comprising:
a fibrous substrate; and
a coating solution including a ligand impregnated to the substrate, the ligand having the formula of claim 7.

10. A metal complex of formula (IV): 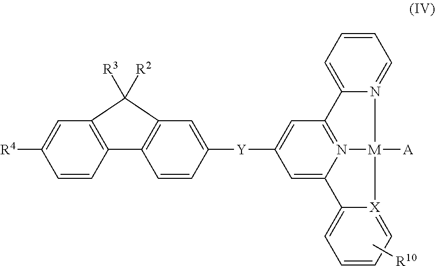
wherein:
R¹⁰ is H or —OR^{a};
R² and R³ are each independently C₁-C₂₄ alkyl, C₂-C₂₄ alkenyl, C₂-C₂₄ alkynyl or —(O—CH₂—CH₂)ₘ—OCH₃;
R⁴ is H, halo, aryl, heterocyclyl, arylalkyl, heterocyclylalkyl, arylalkynyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f};
A is selected from halo, 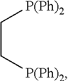
and —C≡C—R^{g};
X═C or N;
each m is independently an integer from 1-12;
each R^{a}, R^{b}, R^{c}, R^{d}, R^{e}, R^{f}, and R^{g} is independently selected from H, C₁-C₂₄ alkyl, C₂-C₂₄ alkenyl or C₂-C₂₄ alkynyl, aryl, heterocyclyl, arylalkyl, arylalkynyl and heterocyclylalkyl; and
Y is a bond, —CH═CH—, —CH═CH-Ph-, or —C≡C—;
M is a metal ion; and
wherein when the complex of formula (IV) bears a charge, it further comprises one or more counterions.

11. An optical-switching device comprising:
a pair of transparent substrates defining a cavity therebetween; and
a nonlinear optical material substantially filling the cavity, wherein the nonlinear optical material includes a metal complex having the formula of claim 10.

12. An organic light-emitting diode, comprising:
an anode;
a cathode; and
an organic compound layer interposed between the anode and the cathode, wherein the organic compound layer includes a metal complex having the formula of claim 10.

13. A chemical sensor comprising:
a fibrous substrate; and
a coating solution including a metal complex impregnated to the substrate, the complex having the formula of claim 10.

14. A metal complex of formula (V): 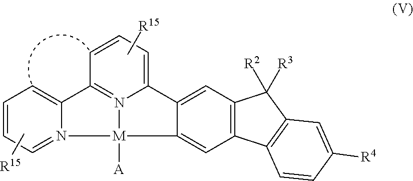
wherein:
the dashed line represents the presence or absence of an optionally substituted aromatic ring or fused aromatic rings;
R² and R³ are each independently C₁-C₂₄ alkyl, C₂-C₂₄ alkenyl, C₂-C₂₄ alkynyl or —(O—CH₂—CH₂)ₘ—OCH₃;
R⁴ is H, halo, aryl, heterocyclyl, arylalkynyl, arylalkyl, arylalkenyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl, or —CH═N—NH—R^{f};
R¹⁵ is each independently selected from H, halo, aryl, heterocyclyl, arylalkynyl, C₁-C₂₄ alkyl, C₂-C₂₄ alkenyl, or C₂-C₂₄ alkynyl;
A is selected from halo and —C≡C—R^{g};
M is a metal ion;
each m is independently an integer from 1-12; and
each R^{b}, R^{c}, R^{d}, R^{e}, R^{f}, and R^{g} is independently selected from H, C₁-C₂₄ alkyl, C₂-C₂₄ alkenyl, C₂-C₂₄ alkynyl, heterocyclyl, arylalkyl, heterocyclylalkyl, arylalkynyl and aryl,
wherein when the complex of formula (V) bears a charge, it further comprises one or more counterions.

15. An optical-switching device comprising:
a pair of transparent substates defining a cavity therebetween; and
a nonlinear optical material substantially filling the cavity, wherein the nonlinear optical material includes a metal complex having the formula of claim 14.

16. An organic light-emitting diode, comprising:
an anode;
a cathode; and
an organic compound layer interposed between the anode and the cathode, wherein the organic compound layer includes a metal complex having the formula of claim 14.

17. A chemical sensor comprising:
a fibrous substrate; and
a coating solution including a metal complex impregnated to the substrate, the complex having the formula of claim 14.

18. A metal complex of formula (VI): 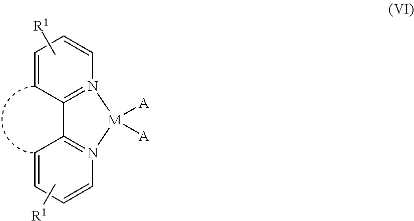
wherein:
the dashed line represents the presence or absence of an optionally substituted aromatic ring or fused aromatic rings;
each R¹ is independently a group of the following formula: 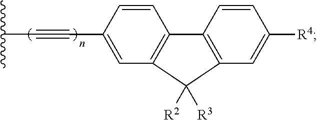
each n equals to 0 or 1 each R² and R³ is independently C₁-C₂₄ alkyl, C₂-C₂₄ alkenyl, C₂-C₂₄ alkynyl, or —(O—CH₂—CH₂)ₘ—OCH₃;
each m is independently an integer from 1-12;
each R⁴ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f}; and
each R^{b}, R^{c}, R^{d}, R^{e}, and R^{f} is independently selected from H, C₁-C₂₄ alkyl, C₂-C₂₄ alkenyl, C₂-C₂₄ alkynyl, heterocyclyl, arylalkyl, heterocyclylalkyl and aryl;
A is selected from halo and —C≡C—R^{g};
each R^{g} is independently selected from H, C₁-C₂₄ alkyl, C₂-C₂₄ alkenyl, C₂-C₂₄ alkynyl, heterocyclyl, arylalkyl, heterocyclylalkyl, arylalkynyl and aryl; and
M is a metal ion;
wherein when the complex of formula (VI) bears a charge, it further comprises one or more counterions.

19. An optical-switching device comprising:
a pair of transparent substrates defining a cavity therebetween; and
a nonlinear optical material substantially filling the cavity, wherein the nonlinear optical material includes a metal complex having the formula of claim 18.

20. An organic light-emitting diode, comprising:
an anode;
a cathode; and
an organic compound layer interposed between the anode and the cathode, wherein the organic compound layer includes a metal complex having the formula of claim 18.

21. A chemical sensor comprising:
a fibrous substrate; and
a coating solution including a metal complex impregnated to the substrate, the complex having the formula of claim 18.

22. A metal complex of formula (VII): 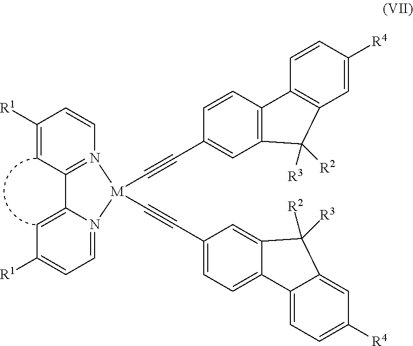
wherein
M is a metal ion;
the dashed line represents the presence or absence of an optionally substituted aromatic ring or fused aromatic rings;
each R¹ is independently C₁-C₂₄ alkyl, C₂-C₂₄ alkenyl, C₂-C₂₄ alkynyl, arylalkynyl or —(O—CH₂—CH₂)ₘ—OCH₃;
each R² and R³ is independently C₁-C₂₄ alkyl, C₂-C₂₄ alkenyl, C₂-C₂₄ alkynyl, or —(O—CH₂—CH₂)ₙ—OCH₃;
each R⁴ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, C₂-C₂₄ alkynyl or —CH═N—NH—R^{f};
each m is independently an integer from 1-12;
each R^{b}, R^{c}, R^{d}, R^{e}, and R^{f} is independently selected from H, C₁-C₂₄ alkyl, C₂-C₂₄ alkenyl, C₂-C₂₄ alkynyl, arylalkyl, heterocyclylalkyl and aryl; and
wherein when the complex of formula (VII) bears a charge, it further comprises one or more counterions.

23. An optical-switching device comprising:
a pair of transparent substrates defining a cavity therebetween; and
a nonlinear optical material substantially filling the cavity, wherein the nonlinear optical material includes a metal complex having the formula of claim 22.

24. An organic light-emitting diode, comprising:
an anode;
a cathode; and
an organic compound layer interposed between the anode and the cathode, wherein the organic compound layer includes a metal complex having the formula of claim 22.

25. A chemical sensor comprising:
a fibrous substrate; and
a coating solution including a metal complex impregnated to the substrate, the complex having the formula of claim 22.

26. A metal complex of formula (VIII): 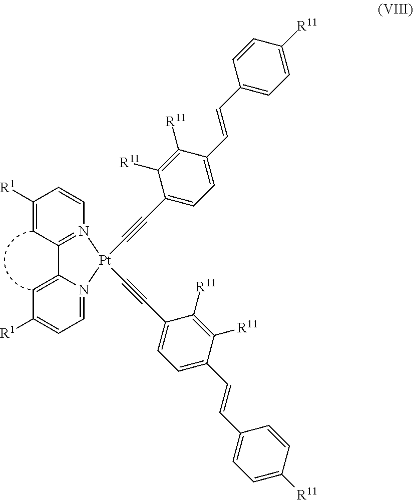
wherein
M is a metal ion;
the dashed line represents the presence or absence of an optionally substituted aromatic ring or fused aromatic rings;
each R¹ is independently C₁-C₂₄ alkyl, C₂-C₂₄ alkenyl, C₂-C₂₄ alkynyl, arylalkynyl or —(O—CH₂—CH₂)ₘ—OCH₃;
each R¹¹ is independently H, halo, aryl, arylalkynyl, heterocyclyl, arylalkyl, arylalkenyl, heterocyclylalkyl, —C(O)R^{b}, —NR^{c}R^{d}, —OR^{e}, —NO₂, —CHO, C₁-C₂₄ alkyl, C₂-C₂₄ alkenyl, and C₂-C₂₄ alkynyl;
each m is independently an integer from 1-12;
each R^{b}, R^{c}, R^{d}, and R^{e} is independently selected from H, C₁-C₂₄ alkyl, C₂-C₂₄ alkenyl, C₂-C₂₄ alkynyl, arylalkyl, heterocyclylalkyl and aryl; and
wherein when the complex of formula (VIII) bears a charge, it further comprises one or more counterions.

27. An optical-switching device comprising:
a pair of transparent substrates defining a cavity therebetween; and
a nonlinear optical material substantially filling the cavity, wherein the nonlinear optical material includes a metal complex having the formula of claim 26.

28. An organic light-emitting diode, comprising:
an anode;
a cathode; and
an organic compound layer interposed between the anode and the cathode, wherein the organic compound layer includes a metal complex having the formula of claim 26.

29. A chemical sensor comprising:
a fibrous substrate; and
a coating solution including a metal complex impregnated to the substrate, the complex having the formula of claim 26.
